(19) United States
(12) Patent Application Publication
(10) Pub. No.: US 2026/0211327 A1
(43) Pub. Date: Jul. 23, 2026

(54) RADIATION-SENSITIVE COMPOSITION AND METHOD FOR FORMING RESIST PATTERN

(71) Applicant: JSR CORPORATION, Tokyo (JP)

(72) Inventors: Takuya OMIYA, Tokyo (JP); Ryo ITO, Tokyo (JP); Atsushi NAKAGAWA, Tokyo (JP); Yuushi MATSUMURA, Tokyo (JP); Katsuaki NISHIKORI, Tokyo (JP)

(73) Assignee: JSR CORPORATION, Tokyo (JP)

(21) Appl. No.: 19/137,710

(22) PCT Filed: Oct. 19, 2023
(86) PCT No.: PCT/JP2023/037922

§ 371 Date: Jun. 11, 2025

(30) Foreign Application Priority Data
Dec. 13, 2022 (JP) 2022-198953

(51) Int. Cl. G03F 7/039; C07C 381/12; C08F 18/12; C08F 22/14; C08F 22/16; C08F 22/22; C08F 22/24; G03F 7/004; H10P 76/20

(52) U.S. Cl. CPC G03F 7/0397; C07C 381/12; C08F 18/12; C08F 22/14; C08F 22/16; C08F 22/22; C08F 22/24; G03F 7/0045; H10P 76/2041

(57) ABSTRACT

A radiation-sensitive composition contains: (A) a polymer having a structural unit represented by formula (1) and; and (B) an onium salt compound which is formed of an organic anion and a cation, in which the organic anion or the cation or both have an iodo group, and which generates an acid through exposure to radiation. In the formula, B¹ represents a single bond or a C≥1 divalent organic group bound to E⁺ at a carbon atom thereof. E⁺ represents a divalent group having an ammonium cation structure or a phosphonium cation structure. B² represents a C≥1 divalent organic group bound to E⁺ and D⁻ at the same carbon atom of different carbon atoms thereof. D⁻ represents a monovalent group having an anion structure.

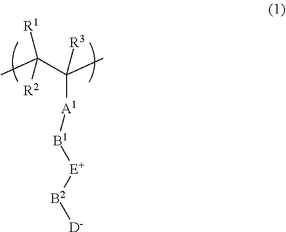

### CROSS-REFERENCE TO RELATED APPLICATIONS

The present application claims a priority from Japanese Patent Application No. 2022-198953 filed on Dec. 13, 2022, the entirety of the disclosure of which is incorporated herein by reference.

### TECHNICAL FIELD

The present disclosure relates to a radiation-sensitive composition and to a method for forming a resist pattern.

### BACKGROUND ART

Photolithographic techniques making use of a resist composition are employed for forming microcircuits of semiconductor elements. In a typical mode of a photolithographic technique, firstly, a film formed from a resist composition (hereinafter may also be referred to as a “resist film”) is exposed to radiation through a mask pattern, to thereby generate an acid. Through a chemical reaction involving the generated acid, a difference in dissolution rate (i.e., dissolution contrast) to a developer between the light-exposed part and the light-unexposed part in the resist film is provided, through which a resist pattern is formed on a substrate.

For example, Patent Document 1 discloses a resist composition containing a resin which includes a repeating unit having an intramolecular salt structure. More specifically, Patent Document 1 discloses that incorporation of a repeating unit having an intramolecular salt structure into the resin, to thereby disperse a quencher uniformly over the resist film in an in-plane direction, whereby an acid is captured in the light-unexposed part.

### PRIOR ART DOCUMENTS

### Patent Documents

Patent Document 1: WO 2017/104355

### SUMMARY OF THE INVENTION

### Problems to be Solved by the Invention

In a rapid progress in further process shrinkage of resist patterns in recent years, attempts have been made to form a pattern having, for example, a line width of 40 nm or less. Thus, when the radiation-sensitive composition for forming a resist film is also employed in formation of such fine resist patterns, the composition must provide a suitable resist pattern at a small dose. In addition, even by use of a high-sensitivity radiation-sensitive composition, variation in line width of a resist pattern may be evoked, when diffusion of an acid generated in the resist film through exposure to light cannot be sufficiently suppressed. Thus, a radiation-sensitive composition for forming a resist film is required to have high sensitivity and excellent LWR (line width roughness) performance.

In the development step, when contact between a developer and a resist film is insufficiently completed, or a residue which has not been dissolved in the developer is deposited on the surface of the pattern, the obtained resist film may be provided with development failures. Such development failures adversely affect the performance of semiconductor devices, as the process shrinkage of a resist pattern proceeds. Therefore, demand has arisen for realization of further process shrinkage of a resist pattern and suppression of occurrence of development failure to a maximum extent as possible.

The disclosure has been made in view of the aforementioned problems. Thus, an object of the present disclosure is to provide a radiation-sensitive composition which can achieve both high sensitivity and excellent LWR performance and which can minimize occurrence of development failure. Another object is to provide a method for forming a resist pattern.

### Means for Solving the Problems

The present inventors have conducted extensive studies to achieve the aforementioned objects, and have found that the aforementioned problems can be solved by a radiation-sensitive composition which contains a polymer having a particular betaine structure and a radiation-sensitive acid-generator having an iodo group. Accordingly, the present disclosure provides the following means.

In one embodiment of the present disclosure, there is provided a radiation-sensitive composition containing (A) a polymer including a structural unit represented by the following formula (1) and (B) an onium salt compound which is formed of an organic anion and a cation, in which the organic anion or the cation or both have an iodo group, and which generates an acid through exposure to radiation.

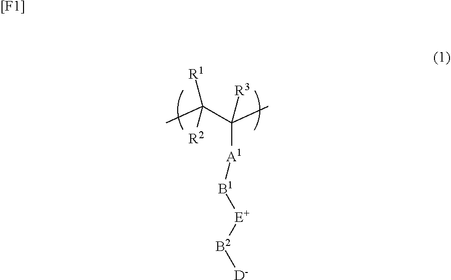

(In the formula (1), each of R¹, R², and R³ independently represents a hydrogen atom, a halogen atom, a hydroxy group, a cyano group, a nitro group, a C1 to C6 alkyl group, or a C1 to C6 haloalkyl group; A¹ represents a single bond, —O—, —CO—, —COO—, —NH—, —CONH—, or *¹—Ar¹-A³-; Ar¹ represents a divalent aromatic ring group; A³ represents a single bond, —O—, —CO—, —COO—, —NH—, or —CONH—; *¹ represents a chemical bond to a carbon atom to which R³ is bound; B¹ represents a single bond or a C≥1 divalent organic group bound to E⁺ in the formula (1) at a carbon atom thereof; E⁺ represents a divalent group having an ammonium cation structure or a phosphonium cation structure; B² represents a C≥1 divalent organic group bound to E⁺ and D⁻ in the formula (1) at the same carbon atom of different carbon atoms thereof; and D⁻ represents a monovalent group having an anion structure.)

In another embodiment of the present disclosure, there is provided a pattern formation method, including a step of forming a resist film on a substrate by applying the aforementioned radiation-sensitive composition onto a substrate, a step of exposing the resist film to light, and a step of developing the light-exposed resist film.

### Advantageous Effects of the Invention

According to the present disclosure, the radiation-sensitive composition contains a polymer including a structural unit represented by the aforementioned formula (1) and an onium salt compound formed of an organic anion and a cation in which the organic anion or the cation or both have an iodo group. As a result, high sensitivity and excellent LWR performance can be achieved in formation of a resist pattern, and occurrence of development failure can be reduced.

### MODES FOR CARRYING OUT THE INVENTION

Hereinafter, carrying out of the present disclosure will be described in detail. In the present specification, the numerical range described with “A to B” refers to include “A” as a lower limit value and “B” as an upper limit value.

### <<Radiation-Sensitive Composition>>

The radiation-sensitive composition of the present disclosure (hereinafter may also be referred to as “the present composition”) contains a polymer which includes a structural unit having a betaine structure (hereinafter may also be referred to as a “polymer (A)”) and a radiation-sensitive acid-generator having an iodo group (hereinafter may also be referred to as a “compound (B)”).

The polymer (A) may form a base resin of the radiation-sensitive composition or form a component other than the base resin. Examples of the component other than the base resin include a polymer having a fluorine atom content (by mass) higher than that of the base resin (hereinafter may also be referred to as a “high-fluorine content polymer”). As used herein, the term “base resin” refers to a polymer component accounting for 50 mass % or more of the entire solid content contained in the present composition. From the viewpoints of achieving high sensitivity and excellent LWR performance in formation of a resist pattern, and excellent effect of reducing development failures, the polymer (A) is preferably a high-fluorine content polymer. Hereinafter, the components contained in the present composition, and optionally incorporated components will be described in detail.

As used herein, the term “hydrocarbon group” encompasses a chain hydrocarbon group, an alicyclic hydrocarbon group, and an aromatic hydrocarbon group. The term “chain hydrocarbon group” refers to a linear-chain hydrocarbon group or a branched hydrocarbon group including one which is composed of only a chain structure and no ring structure. However, the chain hydrocarbon group may be saturated or unsaturated. The term “alicyclic hydrocarbon group” refers to a hydrocarbon group which contains only an alicyclic hydrocarbon moiety as a ring structure and contains no aromatic ring structure. However, the alicyclic hydrocarbon group is not necessarily formed only of an alicyclic hydrocarbon moiety and may contain a chain structure as a partial structure. The term “aromatic hydrocarbon group” refers to a hydrocarbon group which contains an aromatic ring structure as a ring structure. However, the aromatic hydrocarbon group is not necessarily formed only of an aromatic ring structure and may contain a chain structure or an alicyclic hydrocarbon moiety as a partial structure.

The term “aromatic ring group” refers to an n-valent group formed by removing n (n: an integer of 1 or greater) hydrogen atoms from a ring moiety of a substituted or unsubstituted aromatic ring. The term “aromatic ring” encompasses an aromatic hydrocarbon ring and an aromatic heterocycle. The expression “substituted or unsubstituted p-valent hydrocarbon group (p: an integer of 1 or greater)” encompasses a p-valent hydrocarbon group (i.e., an unsubstituted p-valent hydrocarbon group) and a group formed by removing p hydrogen atoms from a hydrocarbon moiety from a substituted hydrocarbon group. Examples of the substituted or unsubstituted p-valent hydrocarbon group include an alkyl group and a fluoroalkyl group (i.e., p=1) and an alkanediyl group and a fluoroalkanediyl group (i.e., p=2). Among them, the fluoroalkyl group corresponds to a “substituted monovalent hydrocarbon group”, and the fluoroalkanediyl group corresponds to a “substituted divalent hydrocarbon group”. The same convention applies to other groups with “substituted or unsubstituted”.

The term “bridged structure” refers to a polycyclic ring structure in which two carbon atoms selected from the carbon atoms forming the ring and not being adjacent to each other are linked by the mediation of a bond linkage chain having one or more carbon atoms. The term “condensed ring structure” refers to a polycyclic ring structure in which a plurality of rings possess a common side (i.e., a bond between two carbon atoms adjacent to each other). The term “spiro ring structure” refers to a polycyclic ring structure formed in a manner in which two rings have a common atom. The spiro ring structure may be formed from a combination of monocyclic structures or may include a bridged structure or a condensed ring structure. The term “organic group” refers to an atomic group formed by removing any hydrogen atom from a carbon-containing compound (i.e., an organic compound). The term “(meth)acryl” encompasses “acryl” and “methacryl”. The term “structural unit” refers to a unit which mainly forming a main-chain (backbone) structure, and two or more of which are included in the main-chain structure.

### <Polymer (A)>

The polymer (A) includes a structural unit represented by the following formula (1) (hereinafter may also be referred to as a “first structural unit”).

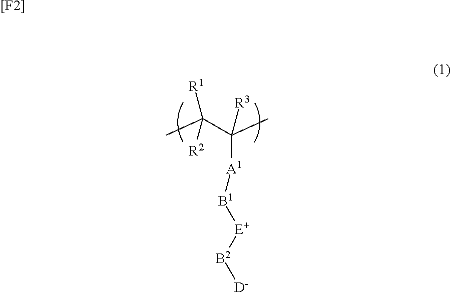

(In the formula (2), each of R¹, R², and R³ independently represents a hydrogen atom, a halogen atom, a hydroxy group, a cyano group, a nitro group, a C1 to C6 alkyl group, or a C1 to C6 haloalkyl group; A¹ represents a single bond, —O—, —CO—, —COO—, —NH—, —CONH—, or *¹—Ar¹-A³-; Ar¹ represents a divalent aromatic ring group; A³ represents a single bond, —O—, —CO—, —COO—, —NH—, or —CONH—; “*¹” represents a chemical bond to a carbon atom to which R³ is bound; B¹ represents a single bond or a C≥1 divalent organic group bound to E⁺ in the formula (1) at a carbon atom thereof; E⁺ represents a divalent group having an ammonium cation structure or a phosphonium cation structure; B² represents a C≥1 divalent organic group bound to E⁺ and D⁻ in the formula (1) at the same carbon atom or different carbon atoms thereof; and D⁻ represents a monovalent group having an anion structure.)

### First Structural Unit

In the aforementioned formula (1), the C1 to C6 alkyl group represented by R¹, R², or R³ may be linear-chain or branched. Examples of the halogen atom represented by R¹, R², or R³, or included in the C1 to C6 haloalkyl group include a fluorine atom, a chlorine atom, a bromine atom, and an iodine atom.

Each of R¹ and R² is preferably, among the aforementioned examples, a hydrogen atom, a halogen atom, a hydroxy group, a cyano group, a nitro group, a C1 to C3 alkyl group, or a C1 to C3 haloalkyl group, with a hydrogen atom being particularly preferred.

From the viewpoint of enhancing co-polymerizability of monomers for forming the first structural unit, R³ is preferably a hydrogen atom or a methyl group.

When A¹ is *¹—Ar¹-A³-, examples of the divalent aromatic ring group represented by Ar¹ include a substituted or unsubstituted phenylene group and a substituted or unsubstituted naphthanylene group. Examples of the substituent include a halogen atom, a hydroxy group, a cyano group, a nitro group, a C1 to C6 alkyl group, and a C1 to C6 haloalkyl group.

When B¹ is C≥1 divalent organic group bound to E⁺ in the aforementioned formula (1) at a carbon atom thereof, and B² is a divalent organic group, examples of the divalent organic group include a C1 to C20 substituted or unsubstituted divalent hydrocarbon group, and a divalent group formed by inserting —O—, —CO—, —COO—, —NH—, or —CONH— into a carbon-carbon bond of a substituted or unsubstituted hydrocarbon group.

Examples of the C1 to C20 divalent hydrocarbon group include a C1 to C20 divalent chain hydrocarbon group, a C3 to C20 divalent alicyclic hydrocarbon group, and a C6 to C20 divalent aromatic hydrocarbon group.

Examples of the C1 to C20 divalent chain hydrocarbon group include a C1 to C20 linear-chain or branched divalent saturated hydrocarbon group, and a C2 to C20 linear-chain or branched divalent unsaturated hydrocarbon group. Among them, a C1 to C20 linear-chain or branched divalent saturated hydrocarbon group is preferred, with a C1 to C10 linear-chain or branched divalent saturated hydrocarbon group being more preferred.

Examples of the C3 to C20 divalent alicyclic hydrocarbon group include groups formed by removing any two hydrogen atoms from a C3 to C20 alicyclic monocyclic hydrocarbon or an alicyclic polycyclic hydrocarbon. Examples of the ring in the alicyclic monocyclic hydrocarbon include saturated aliphatic rings such as cyclobutane, cyclopentane, cyclohexane, cycloheptane, and cyclooctane; and unsaturated aliphatic rings such as cyclobutene, cyclopentene, cyclohexene, cycloheptene, cyclooctene, and cyclodecene. Examples of the ring in alicyclic polycyclic hydrocarbon include saturated aliphatic rings such as norbornane, bicyclo[2.2.2]octane, adamantane, and tricyclo[5.2.1.0^{2,6}]decane; and unsaturated aliphatic rings such as norbornene.

Examples of the C6 to C20 divalent aromatic hydrocarbon group include groups formed by removing any two hydrogen atoms from an aromatic ring (e.g., benzene, naphthalene, anthracene, indene, and fluorene) or a structure in which a chain hydrocarbon or an alicyclic hydrocarbon is bound to the aromatic ring.

When B¹ or B² is a substituted hydrocarbon group, examples of the substituent in B¹ or B² include a halogen atom, a hydroxy group, a cyano group, a nitro group, a C1 to C6 haloalkyl group, a C1 to C6 alkoxy group, and a C2 to C6 alkoxycarbonyl group.

From the viewpoints of enhancing solubility in a developer and ease of synthesizing a monomer forming the first structural unit, B¹ is preferably, among the above examples, a C≥1 divalent organic group bound to E⁺ in the formula (1) at a carbon atom thereof. Also, the divalent organic group represented by B¹ or B² preferably has a chain structure. Specifically, the divalent organic group is preferably a C1 to C20 substituted or unsubstituted divalent chain hydrocarbon group, or a corresponding chain hydrocarbon group in which —O—, —CO—, —COO—, —NH—, or —CONH— is inserted into a carbon-carbon bond thereof. Of these, a C1 to C10 chain structure is more preferred.

Notably, the state of bonding of B¹ to E⁺ at a carbon atom thereof refers to that E⁺ (more specifically, a nitrogen atom or a phosphorus atom in E⁺) is directly bound to a carbon atom in B¹. Also, the bonding state of B² to E⁺ and D⁻ at a carbon atom thereof refers to that E⁺ (more specifically, a nitrogen atom or a phosphorus atom in E⁺) is directly bound to a carbon atom in B², and D⁻ is directly bound to a carbon atom in B². Each of the carbon atom in B¹ bound to E⁺, the carbon atom in B² bound to E⁺, and the carbon atom in B² bound to D⁻ may be any of a primary carbon atom, a secondary carbon atom, and a tertiary carbon atom, and may be located at an position adjacent to a heteroatom-containing group such as an oxygen atom or a carbonyl group in B¹ or B².

E⁺ is a divalent group having an ammonium cation structure or a phosphonium cation structure. Specific examples of preferred divalent groups represented by E⁺ include the structures represented by the following formulas (e-1), (e-2), and (e-3).

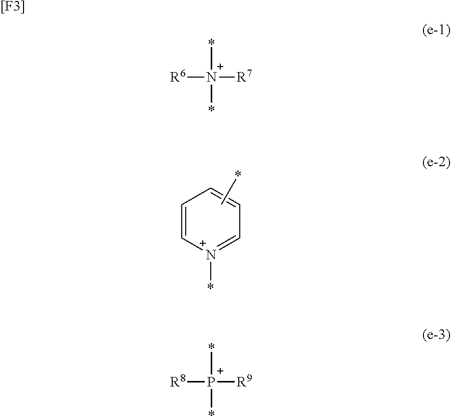

(In the formulas (e-1), (e-2), and (e-3), each of R⁶ and R⁷ independently represents a monovalent hydrocarbon group, or R⁶ and R⁷ are combined to form an aliphatic heterocyclic structure with a nitrogen atom to which R⁶ and R⁷ are bound; each of R⁸ and R⁹ independently represents a monovalent hydrocarbon group, or R⁸ and R⁹ are combined to form an heterocyclic structure with a phosphorus atom to which R⁸ and R⁹ are bound; and * represents a chemical bond.)

In the aforementioned formulas (e-1) to (e-3), examples of the monovalent hydrocarbon group represented by any of R⁶, R⁷, R⁸, and R⁹ include a C1 to C10 monovalent chain hydrocarbon group, a C3 to C20 monovalent alicyclic hydrocarbon group, and a C6 to C20 monovalent aromatic hydrocarbon group.

Examples of the C1 to C10 monovalent chain hydrocarbon group include a C1 to C10 linear-chain or branched saturated hydrocarbon group and a C1 to C10 linear-chain or branched unsaturated hydrocarbon group. Of these, a C1 to C10 linear-chain or branched saturated hydrocarbon group is preferred.

Examples of the C3 to C20 monovalent alicyclic hydrocarbon group include groups formed by removing one hydrogen atom from a C3 to C20 saturated alicyclic hydrocarbon, an unsaturated alicyclic hydrocarbon, or an alicyclic polycyclic hydrocarbon. Specific examples of such alicyclic hydrocarbon include the alicyclic monocyclic hydrocarbons and alicyclic polycyclic hydrocarbons as exemplified in relation to B¹ and B² in the aforementioned formula (1). Examples of the C6 to C20 monovalent aromatic hydrocarbon group include the groups formed by removing one hydrogen atom from the aromatic ring as exemplified in relation to B¹ and B² in the aforementioned formula (1).

Examples of the aliphatic heterocyclic structure formed through combination of R⁶ and R⁷ with a nitrogen atom to which R⁶ and R⁷ are bound include a group formed by extracting a hydrogen atom from a nitrogen atom forming a nitrogen-containing aliphatic heterocycle (e.g., a piperidine ring). Examples of the ring structure formed through combination of R⁸ and R⁹ with a phosphorus atom to which R⁸ and R⁹ are bound include a group formed by extracting a hydrogen atom from a phosphorus atom forming a phosphorus-containing heterocycle (e.g., a phosphinane ring or a phosphol ring). Each of the nitrogen-containing aliphatic heterocyclic structure and the phosphorus-containing heterocyclic structure may have another substituent (e.g., an alkyl group) in the ring thereof.

The divalent group represented by E⁺ preferably has an ammonium cation structure. Among them, the groups represented by the aforementioned formula (e-1) or (e-2) are preferred.

D⁻ is a monovalent group having an anion structure. Specific examples of D⁻ include —COO⁻, —SO₃⁻, —PO₃⁻, —POO⁻, and —O⁻. From the viewpoints of achieving an effect of satisfactorily improving LWR performance and reducing development failure, while achieving high sensitivity of the present composition, D⁻ is preferably, among others, a carboxylate structure (—COO⁻) or a sulfonate structure (—SO₃⁻). Of these, a sulfonate structure is more preferred from the viewpoint of sensitivity, and a carboxylate structure is more preferred from the viewpoint of achieving a satisfactory effect of improving LWR performance and reducing development failure.

The betaine structure in the first structural unit is preferably an intramolecular salt of an ammonium cation structure with a carboxylate structure or a sulfonate structure. Specific examples including preferred members of the first structural unit include the structural units represented by the following formula (1-1) or (1-2).

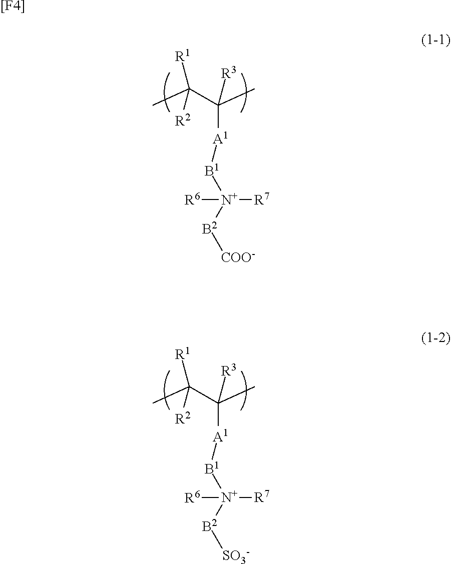

(In the formulas (1-1) and (1-2), R¹, R², R³, A¹, B¹, and B² have the same meanings as those defined in the aforementioned formula (1); and R⁶ and R⁷ have the same meanings as those defined in the aforementioned formula (e-1).)

Specific examples of the first structural unit include the structural units represented by the following formulas.

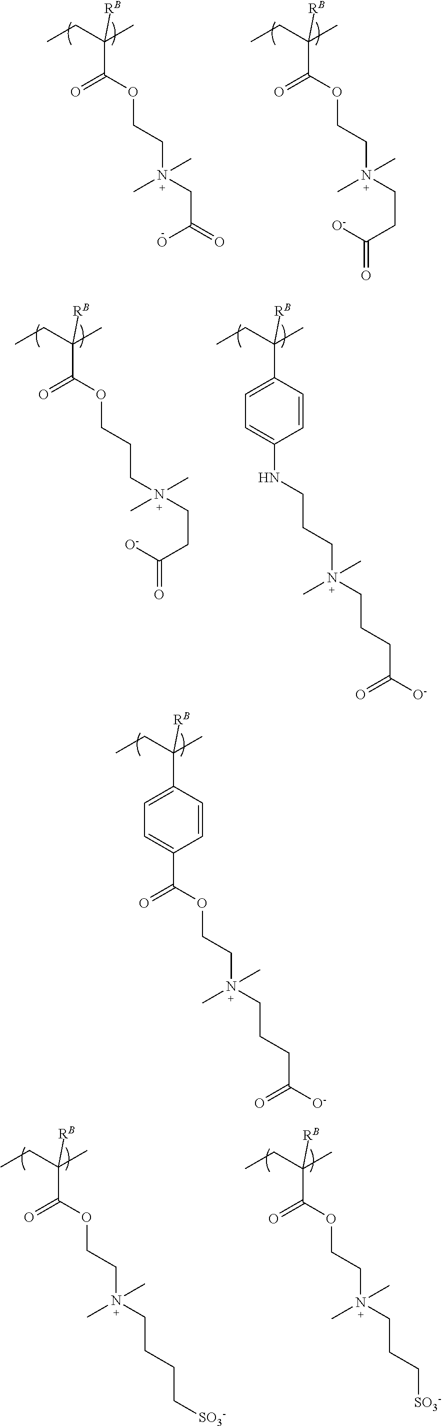

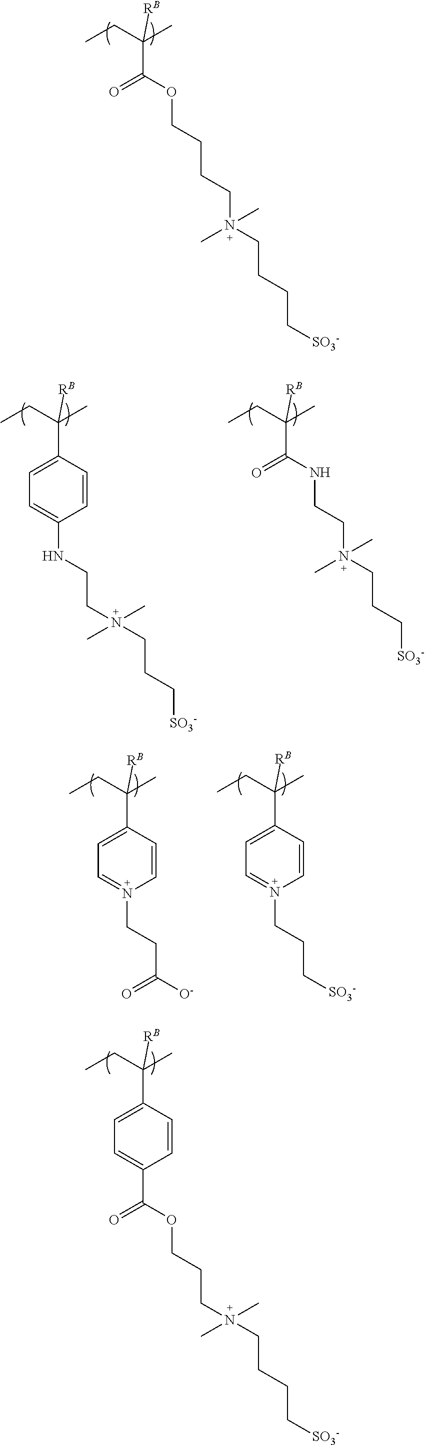

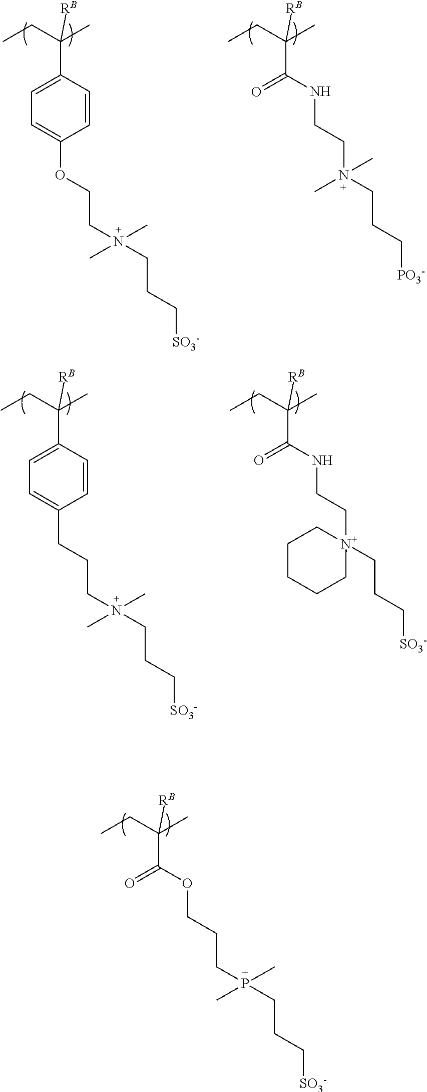

(In the formulas, R^{B} represents a hydrogen atom, a halogen atom, a hydroxy group, a cyano group, a nitro group, a C1 to C6 alkyl group, or a C1 to C6 haloalkyl group.)

From the viewpoint of achieving high sensitivity of the radiation-sensitive composition and an effect of satisfactorily improving LWR performance of the resist film, the relative amount of the first structural unit in the polymer (A) is preferably 1 mol % or more, with respect to all the structural units forming the polymer (A), more preferably 2 mol % or more, still more preferably 5 mol % or more. Also, from the viewpoint of achieving suitable LWR performance of the obtained resist film, the relative amount of the first structural unit in the polymer (A) is preferably 55 mol % or less, with respect to all the structural units forming the polymer (A), more preferably 50 mol % or less, still more preferably 40 mol % or less. By adjusting the first structural unit content to satisfy the above conditions, an effect of reducing occurrence of development failure can be satisfactorily achieved, while high sensitivity and suitable LWR performance are maintained. Notably, the polymer (A) may include the first structural unit singly or in combination of two or more species.

### (Additional Structural Unit)

Along with the first structural unit, the polymer (A) may further include a structural unit differing from the first structural unit (hereinafter may also be referred to as an “additional structural unit”). Examples of the additional structural unit include the following second to fifth structural units.

### Second Structural Unit

The polymer (A) may further include a structural unit having a fluorine atom (hereinafter may also be referred to as a “second structural unit”). Particularly, by incorporating the second structural unit into the polymer (A), to thereby employ the polymer as a high-fluorine content polymer, the polymer (A) may be localized to an upper layer of the resist film, with respect to the base resin. By virtue of the localization, water-repellency of the surface of the resist film during liquid immersion light exposure can be enhanced. Notably, the second structural unit differs from the first structural unit, since the second structural unit has no intramolecular salt structure.

When the polymer (A) is a high-fluorine content polymer, the fluorine atom content of the polymer (A) is preferably 1 mass % or more, more preferably 2 mass % or more, still more preferably 4 mass % or more, particularly more preferably 7 mass % or more. Also, the fluorine atom content of the polymer (A) is preferably 60 mass % or less, more preferably 40 mass % or less, still more preferably 30 mass % or less. The fluorine atom content (mass %) of a polymer can be obtained by determining the structure of the polymer (e.g., a ratio of the carbon atom originating from the monomer forming the second structural unit to the carbon atom originating from the monomer forming the first structural unit) through ¹³C-NMR spectrometry or the like and calculating the content based on the structure determined.

Examples of the second structural unit include a structural unit having a fluorinated aliphatic hydrocarbon structure (hereinafter may also be referred to as a “structural unit (fa)”) and a structural unit having an alkali-soluble group or an alkali-dissociable group and containing a fluorine atom (hereinafter may also be referred to as a “structural unit (fb)”). The term “alkali-dissociable group” refers to a group which is released by the action of alkali, to thereby enhance solubility in an alkaline developer. The polymer (A) may include either the structural unit (fa) or the structural unit (fb), or both the structural unit (fa) and the structural unit (fb). As the second structural unit, at least one species selected from the group consisting of the structural unit (fa) and the structural unit (fb) is preferably employed. Notably, a structural unit having both an alkali-soluble group or an alkali-dissociable group and a fluorinated aliphatic hydrocarbon structure is categorized to the structural unit (fb).

### Structural Unit (fa)

Examples of the structural unit (fa) include the structural units represented by the following formula (7-1). Through incorporation of the structural unit (fa) into the polymer (A), the fluorine atom content of the polymer (A) can be tuned.

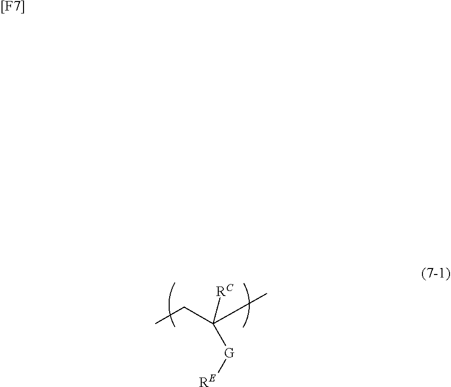

(In the formula (7-1), R^{C} represents a hydrogen atom, a fluoro group, a methyl group, or a trifluoromethyl group; G represents a single bond, an oxygen atom, a sulfur atom, —COO—, —SO₂—O—NH—, —CONH—, or —O—CO—NH—; R^{E} represents a C1 to C20 monovalent fluorinated chain hydrocarbon group; a monovalent group in which a part of the methylene groups of the fluorinated chain hydrocarbon group is substituted by an oxygen atom, a sulfur atom, —COO—, or —CONH—; or a C3 to C20 monovalent fluorinated alicyclic hydrocarbon group.)

In the aforementioned formula (7-1), R^{C} is preferably a hydrogen atom or a methyl group, more preferably a methyl group, from the viewpoint of co-polymerizability of monomers for forming the structural unit (fa). Also, G is preferably a single bond or —COO—, more preferably —COO—, from the viewpoint of co-polymerizability of monomers for forming the structural unit (fa).

Examples of the C1 to C20 monovalent fluorinated chain hydrocarbon group represented by R^{E} include a group in which hydrogen atoms of the C1 to C20 linear-chain or branched alkyl group are partially or totally substituted by a fluorine atom. Examples of the C3 to C20 monovalent fluorinated alicyclic hydrocarbon group represented by R^{E} include a group in which hydrogen atoms of the C3 to C20 monocyclic or polycyclic alicyclic hydrocarbon group (e.g., a group formed by removing one hydrogen atom from an alicyclic monocyclic hydrocarbon or an alicyclic polycyclic hydrocarbon as exemplified in relation to B¹ and B² in the aforementioned formula (1)) are partially or totally substituted by a fluorine atom.

Among them, R^{E} is preferably a monovalent fluorinated chain hydrocarbon group or a monovalent group in which a part of methylene groups of the fluorinated chain hydrocarbon group is substituted by an oxygen atom, a sulfur atom, —COO—, or —CONH—, more preferably a monovalent fluorinated alkyl group or a monovalent group in which a part of methylene groups of the fluorinated alkyl group is substituted by an oxygen atom, a sulfur atom, —COO—, or —CONH—.

Specific examples of the structural unit (fa) include the structural units represented by the following formulas.

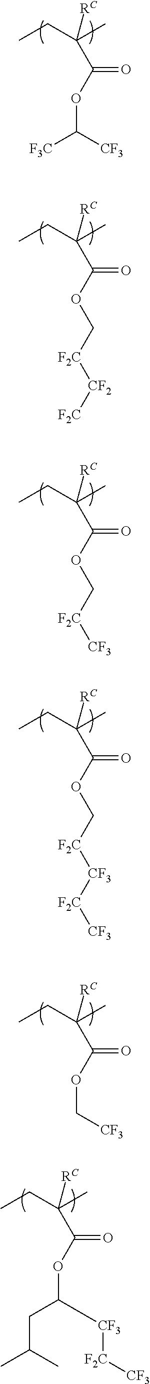

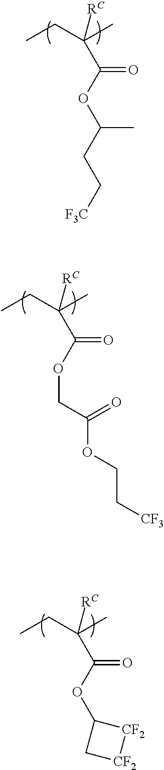

(In the formulas, R^{C} represents a hydrogen atom, a fluoro group, a methyl group, or a trifluoromethyl group.)

When the polymer (A) includes the structural unit (fa), the relative amount of the structural unit (fa), with respect to all the structural units forming the polymer (A), is preferably 30 mol % or more, more preferably 40 mol % or more, still more preferably 50 mol % or more. Also, the relative amount of the structural unit (fa), with respect to all the structural units forming the polymer (A), is preferably 99 mol % or less, more preferably 97 mol % or less, still more preferably 95 mol % or less.

### Structural Unit (fb)

Examples of the structural unit (fb) include the structural units represented by the following formula (7-2). Through incorporation of the structural unit (fb) into the polymer (A), solubility in an alkaline developer is enhanced, while the water-repellency of the resist film during liquid immersion light exposure is enhanced. As a result, occurrence of development failure can be further suppressed.

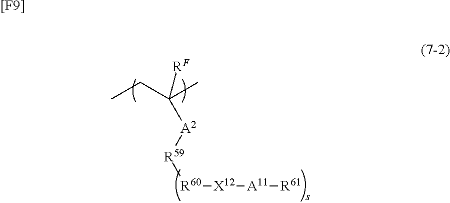

(In the formula (7-2), R^{F} represents a hydrogen atom, a fluoro group, a methyl group, or a trifluoromethyl group; A² represents a single bond, —O—, —CO—, —COO—, —NH—, or —CONH—; R⁵⁹ represents a C1 to C20 (s+1)-valent hydrocarbon group, or a group in which an oxygen atom, a sulfur atom, —NR⁶²—, a carbonyl group, —CO—O—, or —CO—NH— is bound to an R⁶⁰-side end of the hydrocarbon group; R⁶² represents a hydrogen atom or a monovalent organic group; R⁶⁰ represents a single bond or a C1 to C20 divalent organic group; X¹² represents a single bond, a C1 to C20 hydrocarbon group, or a C1 to C20 divalent fluorinated chain hydrocarbon group; A¹¹ represents an oxygen atom, —NR⁶³—, —CO—O—*, or —SO₂—O—*; R⁶³ represents a hydrogen atom or a C1 to C10 monovalent hydrocarbon group; * represents a chemical bond to R⁶¹; R⁶¹ represents a hydrogen atom or a C1 to C30 monovalent organic group; s is an integer of 1 to 3; and, when s is 2 or 3, a plurality of R⁶⁰, X¹², A¹¹, and R⁶¹, individually, are identical to or different from one another.)

When the structural unit (fb) has an alkali-soluble group, R⁶¹ represents a hydrogen atom, and A¹¹ represents an oxygen atom, —CO—O—*, or —SO₂—O—*. X¹² represents a single bond, a C1 to C20 hydrocarbon group, or a C1 to C20 divalent fluorinated chain hydrocarbon group. When A¹¹ is an oxygen atom, X¹² represents a fluorinated hydrocarbon group having a fluorine atom or a fluoroalkyl group on the carbon atom to which A¹¹ is bound. R⁶⁰ represents a single bond or a C1 to C20 divalent organic group. Through incorporation of an alkali-soluble group into the structural unit (fb), affinity to an alkaline developer can be enhanced, and occurrence of development failure can be further suppressed.

The structural unit (fb) having an alkali-soluble group is preferably a structural unit having a hydroxy group bound to a fluorinated saturated chain hydrocarbon structure. More specifically, preferably, in the aforementioned formula (7-2), R⁶¹ is a hydrogen atom; A¹¹ is an oxygen atom, and X¹² is a C1 to C20 divalent fluorinated chain hydrocarbon group. More preferably, X¹² is a fluorinated hydrocarbon group in which a fluorine atom or a fluoroalkyl group is bound to a carbon atom to which A¹¹ has been bound (e.g., a 1,1,1,3,3,3-hexafluoro-2,2-methanediyl group).

When the structural unit (fb) has an alkali-dissociable group, R⁶¹ represents a C1 to C30 monovalent organic group, and A¹¹ represents an oxygen atom, —NR⁶³—, —CO—O—*, or —SO₂—O—*. X¹² represents a single bond or a C1 to C20 divalent fluorinated chain hydrocarbon group. R⁶⁰ represents a single bond or a C1 to C20 divalent organic group. When A¹¹ is —CO—O—* or —SO₂—O—*, a fluorine atom is bound to the carbon atom to which A¹¹ has been bound, or to a carbon atom adjacent thereto, in X¹² or R⁶¹. When A¹¹ is an oxygen atom, each of X¹² and R⁶⁰ is a single bond. R⁵⁹ is a structure in which a carbonyl group is bound to the R⁶⁰-side end of the C1 to C20 hydrocarbon group. R⁶¹ is an organic group having a fluorine atom. In an alternative case, each of X¹² and R⁶⁰ is a single bond; R⁵⁹ is a C1 to C20 hydrocarbon group; R⁶¹ is a structure in which a carbonyl group is bound to the A¹¹-side end, in which a fluorine atom is bound to a carbon atom adjacent to the carbonyl group. By virtue of the presence of an alkali-soluble group in the structural unit (fb), a hydrophobic surface of the resist film changes to a hydrophilic surface in the alkali in an alkali development step. As a result, while water-repellency of the resist film during liquid immersion light exposure is enhanced, affinity between the resist film and the developer can be enhanced during development, whereby development failure can be more efficiently suppressed. In a preferred embodiment of the structural unit (fb) having an alkali-dissociable group, A¹¹ is —CO—O—* or an oxygen atom, and a carboxy group or a hydroxy group is formed by the action of alkali.

Specific examples of the structural unit (fb) include the structural units represented by the following formulas.

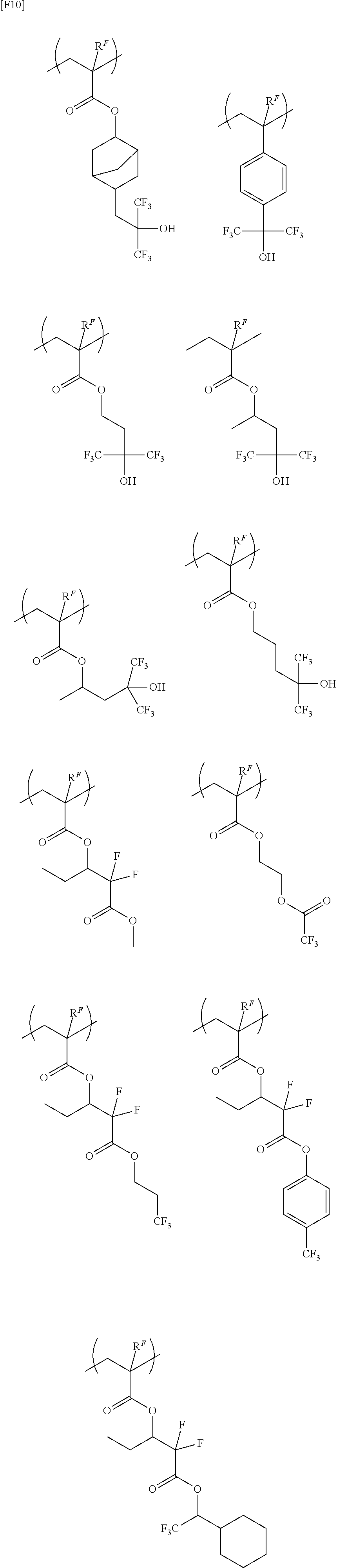

(In the formulas, R^{F} represents a hydrogen atom, a fluoro group, a methyl group, or a trifluoromethyl group.)

When the polymer (A) includes the structural unit (fb), the relative amount of the structural unit (fb), with respect to all the structural units forming the polymer (A), is preferably 30 mol % or more, more preferably 40 mol % or more, still more preferably 50 mol % or more. Also, the relative amount of the structural unit (fb), with respect to all the structural units forming the polymer (A), is preferably 99 mol % or less, more preferably 97 mol % or less, still more preferably 95 mol % or less.

Other than the structural units (fa) and (fb), examples of the second structural unit further include a structural unit having a fluorine atom and an acid-dissociable group. Notably, in the present specification, a structural unit having a fluorine atom (excepting a fluorine atom directly bound to a main chain and a fluorine atom in the trifluoromethyl group) and an acid-dissociable group is categorized to the second structural unit. From the viewpoint of achieving more enhanced effect of suppressing occurrence of development failure, the polymer (A) preferably include a structural unit (fb) as a second structural unit.

When the polymer (A) is a high-fluorine content polymer, the relative amount of the second structural unit in the polymer (A), with respect to all the structural units forming the polymer (A), is preferably 45 mol % or more, more preferably 50 mol % or more, still more preferably 60 mol % or more. Also, when the polymer (A) is a high-fluorine content polymer, the relative amount of the second structural unit in the polymer (A), with respect to all the structural units forming the polymer (A), is preferably 99 mol % or less, more preferably 97 mol % or less, still more preferably 95 mol % or less. By adjusting the second structural unit content to satisfy the aforementioned conditions, an effect of reducing occurrence of development failure can be satisfactorily achieved, while water-repellency of the resist film is sufficiently enhanced during liquid immersion light exposure.

When the polymer (A) is a base resin, the relative amount of the second structural unit in the polymer (A), with respect to all the structural units forming the polymer (A), is preferably 40 mol % or less, more preferably 30 mol % or less, still more preferably 20 mol % or less. Notably, the polymer (A) may include the second structural unit singly or in combination of two or more species.

### Third Structural Unit

The polymer (A) may further include a structural unit having an acid-dissociable group (excepting a structural unit belonging to the first structural unit or the second structural unit) (hereinafter may also be referred to as a “third structural unit”). As used herein, the acid-dissociable group refers to a group that can substitute a hydrogen atom of an acidic group such as a carboxy group or a hydroxy group, and which is dissociated by the action of acid. Through incorporation of the third structural unit into the polymer (A), the acid-dissociable group is dissociated by the acid generated from the present composition upon exposure to light, to thereby provide an acidic group. As a result, solubility of a polymer component in a developer is modified. Thus, the present composition can be endowed with suitable lithographic characteristics.

No particular limitation is imposed on the third structural unit, so long as it has an acid-dissociable group. Examples of the third structural unit include the structural units represented by the following formula (i-1) (hereinafter may also be referred to as “structural units (3-1)”), and the structural units represented by the following formula (i-2) (hereinafter may also be referred to as “structural units (3-2)”).

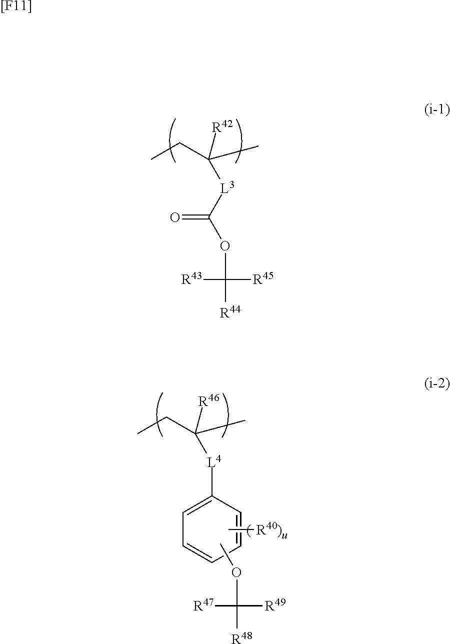

(In the formula (i-1), R⁴² represents a hydrogen atom, a fluorine atom, a methyl group, or a trifluoromethyl group; L³ represents a single bond, a substituted or unsubstituted phenylene group, **—COO—Ar¹—, or **—CONH—Ar¹—; Ar¹ represents a substituted or unsubstituted phenylene group; ** represents a chemical bond to a carbon atom which R⁴² is bound; R⁴³ represents a hydrogen atom or a C1 to C20 monovalent hydrocarbon group; each of R⁴⁴ and R⁴⁵ independently represents a C1 to C20 monovalent hydrocarbon group, a monovalent aromatic heterocyclic group, or a C3 to C20 alicyclic hydrocarbon structure formed by combining R⁴⁴ and R⁴⁵ with each other by the mediation of a carbon atom to which R⁴⁴ and R⁴⁵ are bound; when R⁴³ is a hydrogen atom, either R⁴⁴ or R⁴⁵ or both of them independently represent a monovalent unsaturated hydrocarbon group, a monovalent aromatic heterocyclic group, or a C3 to C20 alicyclic unsaturated hydrocarbon structure formed by combining R⁴⁴ and R⁴⁵ with each other by the mediation of a carbon atom to which R⁴⁴ and R⁴⁵ are bound; and at least a part of hydrogen atoms of R⁴³, R⁴⁴, and R⁴⁵ may be substituted by a halogen atom or an alkoxy group; and
in the formula (i-2), R⁴⁶ represents a hydrogen atom, a fluorine atom, a methyl group, or a trifluoromethyl group; L⁴ represents a single bond, —COO—, or —CONH—; each of R⁴⁷, R⁴⁸, and R⁴⁹ independently represents a hydrogen atom, a C1 to C20 monovalent hydrocarbon group, or a C1 to C20 monovalent oxyhydrocarbon group; R⁴⁰ represents a hydroxy group, a C1 to C10 monovalent hydrocarbon group, or a C1 to C10 oxyhydrocarbon group; u is an integer of 0 to 4; and at least a part of hydrogen atoms of R⁴⁷, R⁴⁸, and R⁴⁹ may be substituted by a halogen atom or an alkoxy group.)

In the aforementioned formula (i-1), R⁴² is preferably a hydrogen atom or a methyl group, more preferably a methyl group, from the viewpoint of co-polymerizability of monomers for forming a structural unit (3-1). In the aforementioned formula (i-2), R⁴⁶ is preferably a hydrogen atom, from the viewpoint of co-polymerizability of monomers for forming a structural unit (3-2).

Examples of the C1 to C20 monovalent hydrocarbon group represented by any of R⁴³ to R⁴⁵, and R⁴⁷ to R⁴⁹ include a C1 to C20 monovalent chain hydrocarbon group, a C3 to C20 monovalent alicyclic hydrocarbon group, and a C6 to C20 monovalent aromatic hydrocarbon group. Specific examples thereof include monovalent hydrocarbon groups corresponding to the C1 to C20 divalent hydrocarbon groups as exemplified in relation to B¹ and B² in the aforementioned formula (1). Examples of the monovalent unsaturated hydrocarbon group represented by R⁴⁴ or R⁴⁵ include the monocyclic or polycyclic alicyclic unsaturated hydrocarbon groups and aromatic hydrocarbon groups, as exemplified in relation to B¹ and B² in the aforementioned formula (1). Examples of monovalent aromatic heterocyclic groups include a furyl group and a thienyl group.

The C3 to C20 alicyclic hydrocarbon structure formed by combining R⁴⁴ and R⁴⁵ with each other by the mediation of a carbon atom to which R⁴⁴ and R⁴⁵ are bound may be saturated or unsaturated. Examples of the alicyclic hydrocarbon structure include groups formed by removing one hydrogen atom from each of the alicyclic monocyclic hydrocarbons and alicyclic polycyclic hydrocarbons, as exemplified in relation to B¹ and B² in the aforementioned formula (1). Notably, to a ring of the alicyclic hydrocarbon structure, a group such as an alkyl group, an alkoxy group, a halogen atom (e.g., a fluorine atom, a chlorine atom, a bromine atom, or an iodine atom), or the like may be bonded.

Examples of the C1 to C20 monovalent oxyhydrocarbon group represented by any of R⁴⁷ to R⁴⁹ include groups formed by incorporating one oxygen atom into the chemical bond-side end of each of the C1 to C20 monovalent hydrocarbon groups, as exemplified in relation to the aforementioned R⁴³ to R⁴⁵ and R⁴⁷ to R⁴⁹.

R⁴⁷ to R⁴⁹ are preferably a chain hydrocarbon group and a cycloalkyloxy group, among the above examples.

In the substituted phenylene group represented by L³ or and Ar¹, examples of the substituent incorporated into the phenylene group include a hydroxy group, a C1 to C10 monovalent hydrocarbon group, a C1 to C10 oxyhydrocarbon group, an acyl group, and an acyloxy group.

Specific examples of the structural unit (3-1) include the structural units represented by the following formulas.

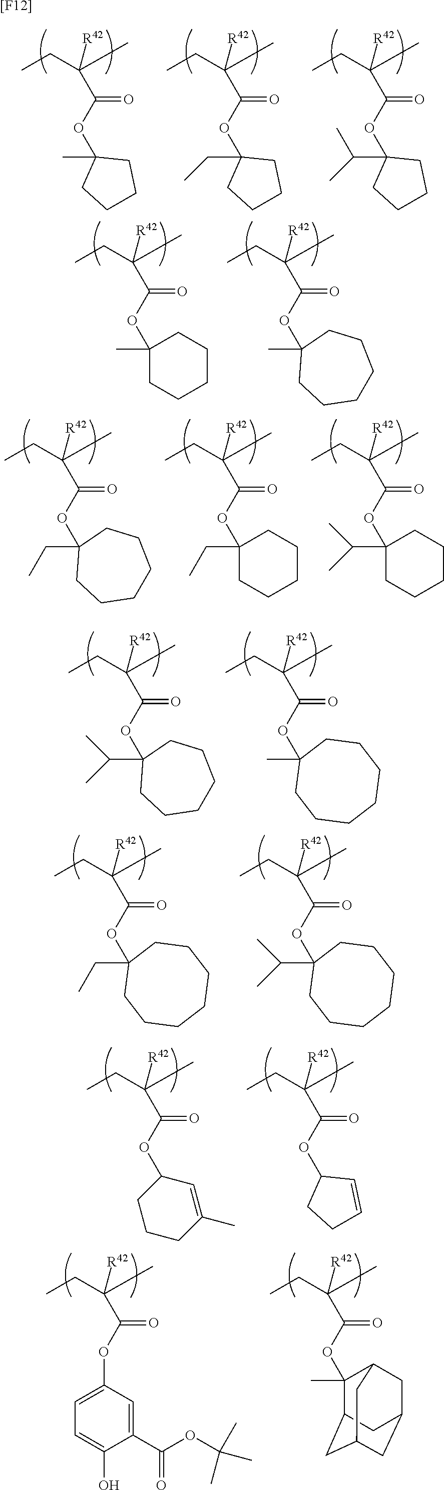

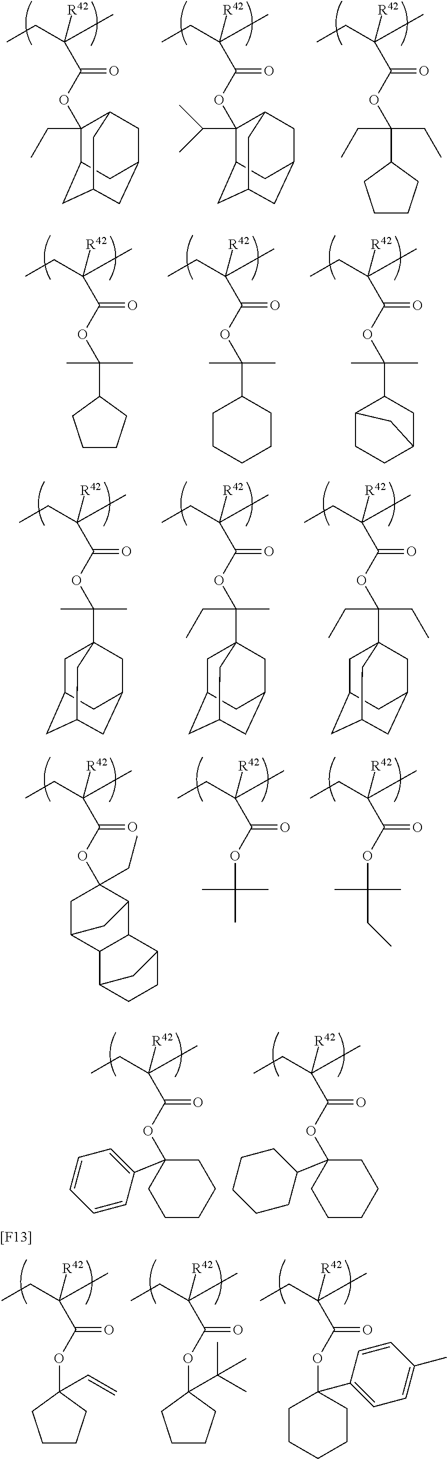

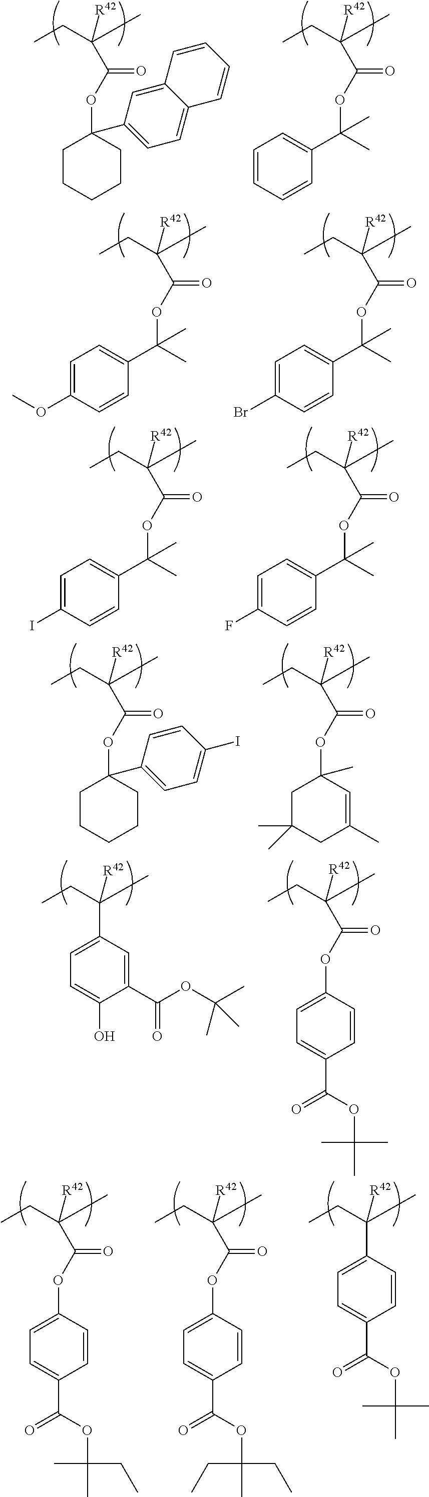

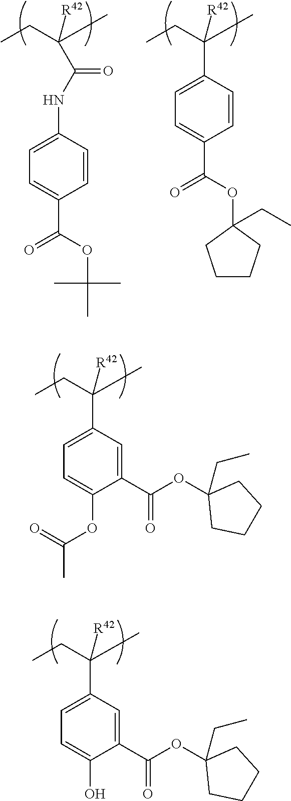

(In the above formulas, R⁴² represents a hydrogen atom, a fluorine atom, a methyl group, or a trifluoromethyl group.)

Specific examples of the structural unit (3-2) include the structural units represented by the following formulas.

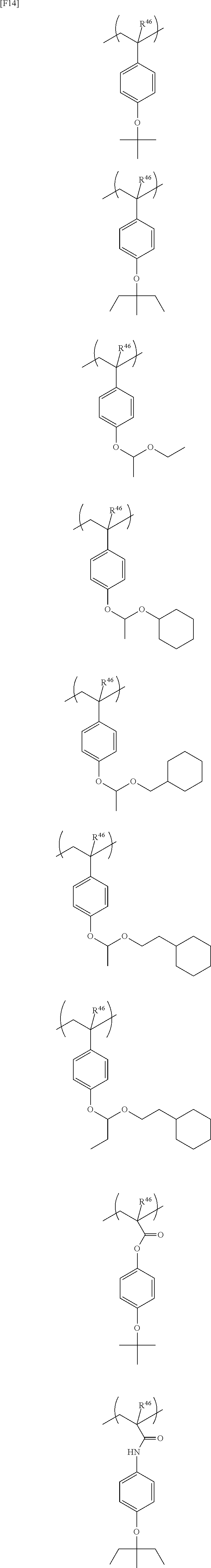

(In the above formulas, R⁴⁶ represents a hydrogen atom, a fluorine atom, a methyl group, or a trifluoromethyl group.)

When the polymer (A) include the third structural unit, the relative amount of the third structural unit, with respect to all the structural units forming the polymer (A), is preferably 2 mol % or more, more preferably 5 mol % or more, still more preferably 10 mol % or more. Also, the relative amount of the third structural unit, with respect to all the structural units forming the polymer (A), is preferably 80 mol % or less, more preferably 70 mol % or less, still more preferably 50 mol % or less. By adjusting the third structural unit content to satisfy the aforementioned conditions, the difference in dissolution rate to a developer between the light-exposed part and the light-unexposed part can be sufficiently elevated, to thereby provide a resist film with a suitable pattern shape, which is preferred. Notably, the polymer (A) may include the third structural unit singly or in combination of two or more species.

### Fourth Structural Unit

The polymer (A) may further include a structural unit having a hydroxy group bound to an aromatic ring (excepting a structural unit belonging to any of the first to third structural units) (hereinafter may also be referred to as a “fourth structural unit”). Through incorporation of the fourth structural unit into the polymer (A), etching resistance and difference in solubility in a developer between the light-exposed part and the light-unexposed part (i.e., dissolution contrast) can be enhanced, which is preferred. Particularly in pattern formation through exposure to a radiation having a wavelength 50 m or shorter, such as an electron beam or EUV, a polymer including the fourth structural unit is preferably employed.

In the fourth structural unit, examples of the aromatic ring to which a hydroxy group is bound include a benzene ring, a naphthalene ring, and an anthracene ring. Of these, a benzene ring or a naphthalene ring is preferred, with a benzene ring being more preferred. Also, in the fourth structural unit, no particular limitation is imposed on the number and positions of the hydroxy group(s) bound to an aromatic ring. The number of the hydroxy group(s) bound to an aromatic ring is preferably 1 to 3, more preferably 1 or 2. Examples of the fourth structural unit include the structural units represented by the following formula (ii).

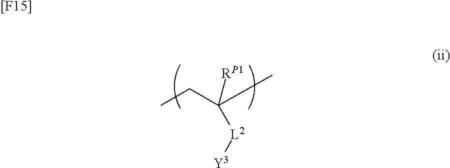

(In the formula (ii), R^{P1} represents a hydrogen atom, a fluorine atom, a methyl group, or a trifluoromethyl group; L² represents a single bond, —O—, —CO—, —COO—, or —CONH—; Y³ represents a monovalent group having a hydroxy group bound to an aromatic ring.)

In the aforementioned formula (ii), R^{P1} is preferably a hydrogen atom or a methyl group, from the viewpoint of co-polymerizability of monomers for forming the fourth structural unit. L² is preferably a single bond or —COO—.

Specific examples of the fourth structural unit include the structural units represented by the following formulas.

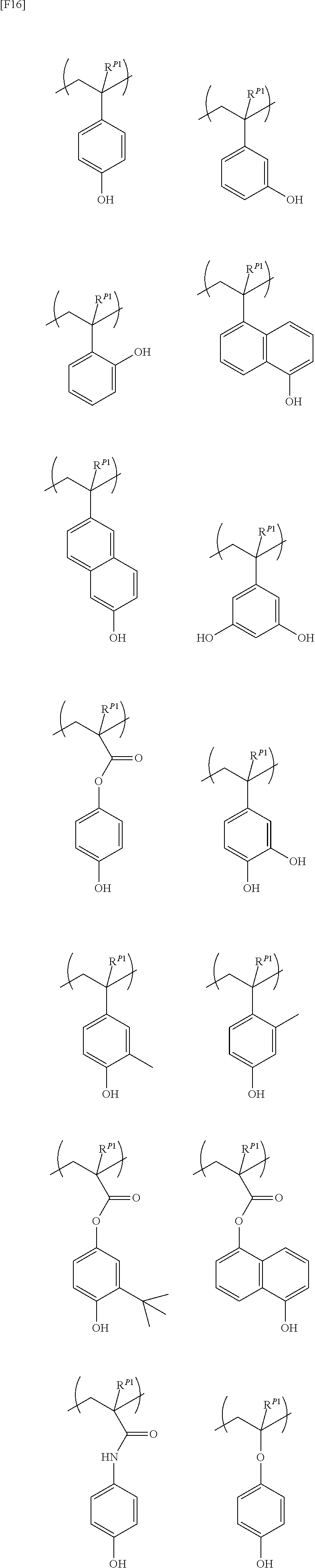

(In the formulas, R^{P1} represents a hydrogen atom, a fluorine atom, a methyl group, or a trifluoromethyl group.)

When the polymer (A) include the fourth structural unit, the relative amount of the fourth structural unit, with respect to all the structural units forming the polymer (A), is preferably 2 mol % or more, more preferably 5 mol % or more, still more preferably 10 mol % or more. Also, the relative amount of the fourth structural unit, with respect to all the structural units forming the polymer (A), is preferably 80 mol % or less, more preferably 70 mol % or less, still more preferably 60 mol % or less. Notably, the polymer (A) may include the fourth structural unit singly or in combination of two or more species. The polymer (A) may include a structural unit in which an acid-dissociable group and a hydroxy group are bound to the same aromatic ring or different aromatic rings. In the present specification, the structural unit in which an acid-dissociable group and a hydroxy group are bound to the same aromatic ring or different aromatic rings is categorized to the third structural unit.

### Fifth Structural Unit

The polymer (A) may further include a structural unit having a lactone structure, a cyclic carbonate structure, a sultone structure, or a combination of two or more of them (hereinafter may also be referred to as a “fifth structural unit”). Through incorporation of the fifth structural unit into the polymer (A), solubility of the polymer in a developer can be tuned, whereby lithographic characteristics of the present composition can be further improved, which is preferred. In addition, through incorporation of the fifth structural unit into the polymer (A), close adhesion of the resist film obtained from the present composition with a substrate can be improved.

Examples of the fifth structural unit include those represented by the following formulas.

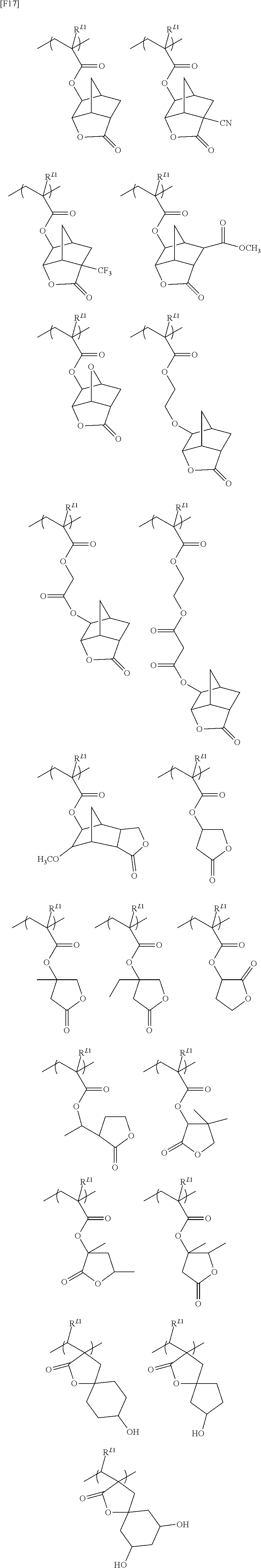

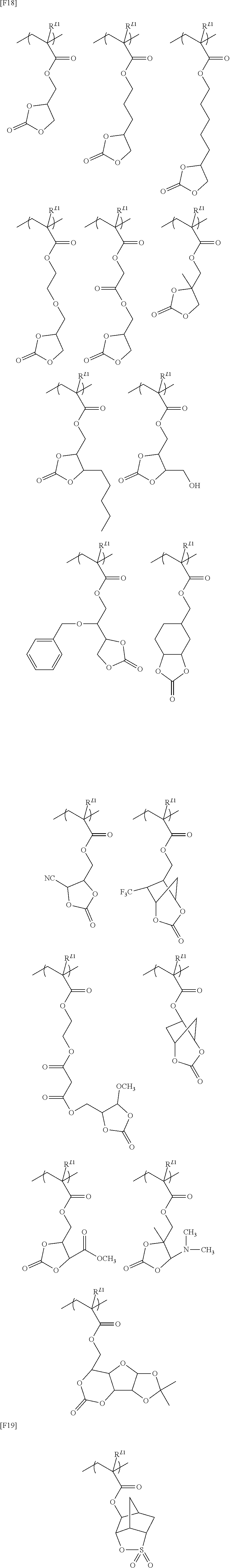

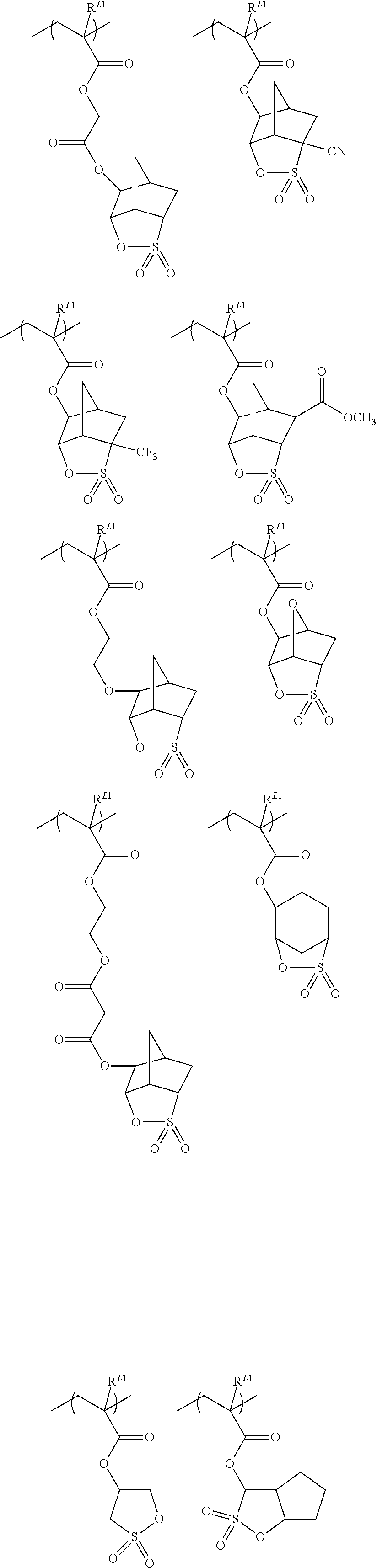

(In the formulas, R^{L1} represents a hydrogen atom, a fluorine atom, a methyl group, or a trifluoromethyl group.)

When the polymer (A) include the fifth structural unit, the relative amount of the fifth structural unit, with respect to all the structural units forming the polymer (A), is preferably 1 mol % or more, more preferably 3 mol % or more, still more preferably 5 mol % or more. Also, the relative amount of the fifth structural unit, with respect to all the structural units forming the polymer (A), is preferably 50 mol % or less, more preferably 40 mol % or less, still more preferably 30 mol % or less. By adjusting the fifth structural unit content of the polymer (A) to satisfy the aforementioned conditions, lithographic characteristics of the present composition can be improved, and close adhesion of the resist film obtained from the present composition with a substrate can be improved, which is preferred. Notably, the polymer (A) may include the fifth structural unit singly or in combination of two or more species.

In addition to the aforementioned examples, examples of the additional structural unit include the following structural units. So long as the effects of the present disclosure are not impaired, the relative amount of the relevant structural unit may be appropriately predetermined depending on the type of the structural unit.
Structural unit having an alcoholic hydroxy group (excepting any of the first to fifth structural units)
Structural unit having a partial structure that can generate an acid in the present composition through exposure to light (e.g., a structural unit having a partial structure formed of a triarylsulfonium cation and an organic anion, or a structural unit having a partial structure formed of a diaryliodonium cation and an organic anion)
Structural unit having a cyano group, a nitro group, or a sulfonamide group (e.g., a structural unit derived from 2-cyanomethyladamantan-2-yil (meth)acrylate)
Structural unit having a non-acid-dissociable hydrocarbon group (e.g., a structural unit derived from styrene, a structural unit derived from vinylnaphthalene, a structural unit derived from n-pentyl (meth)acrylate, or a structural unit derived from indene)

Examples of the structural unit having a having a partial structure that can generate an acid in the present composition through exposure to light (hereinafter may also be referred to as a “sixth structural unit”) include a structural unit having a partial structure that can generate a sulfonic acid (including a sulfonate group) in the present composition through exposure to light, and a structural unit having a partial structure that can generate a carboxylic acid (including a carboxylate group) in the present composition through exposure to light. Among them, the sixth structural unit is preferably a structural unit in which a sulfonate anion (—SO₃—) is bound to a main chain of the polymer by the mediation of a liking group, and a radiation-sensitive onium cation forms a counter ion (referred to as a “structural unit (6-1)”), or a structural unit in which a carboxylate anion (—CO₂—) is bound to a main chain of the polymer by the mediation of a liking group, and a radiation-sensitive onium cation forms a counter ion (referred to as a “structural unit (6-2)”). Also, the polymer(A) may further include, as a sixth structural unit, the structural unit (6-1) and the structural unit (6-2). From the viewpoint of further enhancing the sensitivity of the present composition, the sixth structural unit preferably has an iodo group.

The weight average molecular weight (Mw) of the polymer (A), which is determined through gel permeation chromatography (GPC) and is reduced to polystyrene, is preferably 1,000 or more, more preferably 2,000 or more, still more preferably 3,000 or more, particularly preferably 4,000 or more. Also, the Mw of the polymer (A) is preferably 50,000 or less, more preferably 30,000 or less, still more preferably 20,000 or less, particularly preferably 18,000 or less. Adjusting the Mw of the polymer (A) so as to satisfy the above conditions is preferred, since coatability of the present composition and heat resistance of the formed resist film can be improved, and development failure can be sufficiently suppressed.

The ratio (Mw/Mn) of Mw to the number average molecular weight (Mn) of the polymer (A), which is determined through GPC and is reduced to polystyrene, is preferably 5.0 or less, more preferably 3.0 or less, still more preferably 2.0 or less. Also, the Mw/Mn of the polymer (A) is generally 1.0 or greater.

When the polymer (A) is used as a high-fluorine content polymer, the polymer (A) content of the present composition, with respect to the total solid content of the present composition (i.e., the total amount of the composition excepting the solvent contained in the present composition), is preferably 0.1 mass % or higher, more preferably 0.5 mass % or higher, still more preferably 1 mass % or higher. Also, the polymer (A) content, with respect to the total solid content of the present composition, is preferably 20 mass % or lower, more preferably 15 mass % or lower, still more preferably 12 mass % or lower.

When the polymer (A) is used as a base resin, the polymer (A) content of the present composition, with respect to the total solid content of the present composition (i.e., the total amount of the composition excepting the solvent contained in the present composition), is preferably 70 mass % or higher, more preferably 75 mass % or higher, still more preferably 80 mass % or higher. Also, the polymer (A) content, with respect to the total solid content of the present composition, is preferably 99 mass % or lower, more preferably 98 mass % or lower, still more preferably 95 mass % or lower. The polymer (A) may be used singly or in combination of two or more species.

Notably, no particular limitation is imposed on the method of synthesizing the polymer (A). In one possible synthesis method, monomers for forming respective structural units are polymerized in an appropriate solvent in the presence of a radical polymerization initiator or the like.

### <Compound (B)>

The compound (B) is an onium salt formed of a cation and an organic anion. One or both of the cation and the organic anion forming the onium salt has an iodo group. In the present composition, the compound (B) serves as a radiation-sensitive acid-generator.

The organic anion forming the onium salt is generally an anion formed by removing a proton from an acid residue of an organic acid. The onium salt compound serving as a radiation-sensitive acid-generator releases an organic anion through the action of radiation via decomposition of the radiation-sensitive onium cation. The thus-released organic anion is bound to hydrogen extracted from a component of the present composition (e.g., a radiation-sensitive acid-generator itself or a solvent), to thereby generate an acid derived from the organic anion.

The compound (B) may be incorporated into the present composition as a radiation-sensitive acid-generating agent, into the present composition as a an acid diffusion control agent (more specifically, a light-degradable base), or into the present composition serving as both an acid-generating agent and an acid diffusion control agent. As used herein, the term “acid-generating agent” refers to a component for generating an acid (strong acid) which allows an acid-dissociable group included in the component in the radiation-sensitive composition to be released from the component upon exposure to light. The “acid diffusion control agent” is a component that can suppress diffusion of an acid generated in the resist film via light exposure originating from the acid-generating agent, whereby chemical reaction by the acid in the light-unexposed part is suppressed.

When the present composition contains two or more onium salt compounds as radiation-sensitive acid-generators, these onium salt compounds may be classified into an acid-generating agent and an acid diffusion control agent, depending on the relative strength of the acid. The degree of acidity can be evaluated on the basis of acid dissociation constant (pKa). The acid dissociation constant of the acid generated by the action of the light-degradable base is preferably −3 or higher, more preferably −1 to 7, still more preferably 0 to 5.

Notably, hereinafter, the radiation-sensitive acid-generating agent in which one or both of the cation and the organic anion have an iodo group may be referred to as an “acid-generating agent (B¹)”, and the acid diffusion control agent in which one or both of the cation and the organic anion have an iodo group may be referred to as a “light-degradable base (B²)”.

The present composition preferably contains both an acid-generating agent and a light-degradable base, as radiation-sensitive acid-generators. At least one of the acid-generating agent and the light-degradable base contained in the present composition essentially includes the compound (B). Specific modes of the radiation-sensitive acid-generator(s) contained in the present composition are the following modes 1 to 3.
[Mode 1] Containing the acid-generating agent (B1) and an onium salt compound which can generate an acid having an acidity lower than that of an acid generated by the acid-generating agent (B1) and which differs from the compound (B) (hereinafter may also be referred to as an “additional light-degradable base”).
[Mode 2] Containing the light-degradable base (B2) and an onium salt compound which can generate an acid having an acidity higher than that of an acid generated by the light-degradable base (B2) and which differs from the compound (B) (hereinafter may also be referred to as an “additional acid-generating agent”).
[Mode 3] Containing, as compounds (B), the acid-generating agent (B1) (i.e., a first onium salt compound), and the light-degradable base (B2) (i.e., a second onium salt compound which can generate an acid having an acidity lower than that of the acid generated by the first onium salt compound).
Through employment of any of the above modes, there can be provided a resist film in which exhibits excellent LWR performance and in which occurrence of development failure is reduced, while the sensitivity of the radiation-sensitive composition is enhanced.

### Structure of Compound (B)

In the compound (B), at least one of the cation and the organic anion essentially has an iodo group. Thus, the compound (B) may be an onium salt in which the cation has an iodo group and the organic anion has no iodo group, or an onium salt in which the organic anion has an iodo group and the cation has no iodo group. Alternatively, the compound (B) may be an onium salt in which both the cation and the organic anion each have an iodo group. The compound (B) may contain one single species or two or more species in combination.

The number of iodo groups in the compound (B) is essentially 1 or greater. From the viewpoint of enhancing the sensitivity of the radiation-sensitive composition and LWR performance of the obtained resist film, the total number of the iodo groups in the compound (B) is more preferably 2 or greater. From the viewpoint of achieving sensitivity, LWR performance, and ease of synthesis in a balanced manner, the number of the iodo groups in the compound (B) is preferably 10 or smaller, more preferably 8 or smaller.

No particular limitation is imposed on the position of bonding of an iodo group in the compound (B). From the viewpoint of yielding a radiation-sensitive composition with higher sensitivity, the compound (B) preferably has a structure in which an iodo group is bound to an aromatic ring. When the compound (B) has a plurality of iodo groups, those iodo groups may be bound to the same aromatic ring or different aromatic rings in the compound (B), respectively. The aromatic ring to which an iodo group or iodo groups are bound is preferably a benzene ring or a naphthalene ring, more preferably a benzene ring.

The compound (B) is preferably a compound that can generate a sulfonic acid, a carboxylic acid, or a sulfonamide in the composition upon exposure to light, more preferably a compound that can generate a sulfonic acid or a carboxylic acid. Specific examples of the compound (B) include the onium salts represented by the following formula (2) or (3).

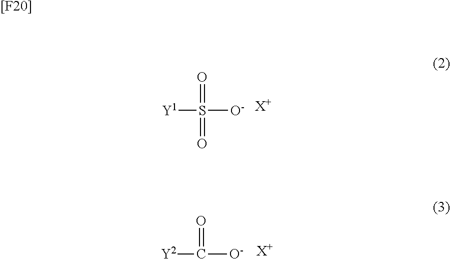

(In the formulas (2) and (3), each of Y¹ and Y² represents a C1 to C40 monovalent organic group; X⁺ represents a monovalent onium cation; in the formula (2), Y¹ or X⁺ or both have an iodo group; and, in the formula (3), Y² or X⁺ or both have an iodo group.)

In the aforementioned formulas (2) and (3), the C1 to C40 monovalent organic group represented by Y¹ or Y² may be a group having a chain structure (hereinafter may also be referred to as a “chain organic group”) or a group having a cyclic structure.

When the monovalent organic group represented by Y¹ or Y² is a chain organic group, examples of the chain organic group include a C1 to C40 linear-chain or branched saturated hydrocarbon group, a C1 to C40 linear-chain or branched unsaturated hydrocarbon group, a C2 to C40 monovalent group formed by inserting a (thio)ether group or an ester group into a carbon-carbon bond of a linear-chain or branched hydrocarbon group; and a C1 to C40 monovalent group in which any hydrogen atom of the monovalent group or a linear-chain or branched hydrocarbon group is substituted. Examples of the substituent include a halogen atom (e.g., a fluorine atom, a chlorine atom, a bromine atom, and an iodine atom), a hydroxy group, and a nitro group.

When the monovalent organic group represented by Y¹ or Y² is a group having a cyclic structure, examples of the cyclic structure in Y¹ or Y² include a C3 to C20 alicyclic hydrocarbon structure, a C3 to C20 aliphatic heterocyclic structure, and a C6 to C20 aromatic ring structure. Those cyclic structures may have a substituent. Examples of the substituent include an alkoxy group, an alkoxycarbonyl group, an alkoxycarbonyloxy group, a halogen atom (e.g., a fluorine atom, a chlorine atom, a bromine atom, and an iodine atom), a hydroxy group, and, and oxo group.

Examples of the C3 to C20 alicyclic hydrocarbon structure include a C3 to C20 alicyclic monocyclic hydrocarbon structure and a C6 to C20 alicyclic polycyclic hydrocarbon structure. The C3 to C20 alicyclic monocyclic hydrocarbon structure and the C6 to C20 alicyclic polycyclic hydrocarbon structure may be saturated or unsaturated. Also, the alicyclic polycyclic structure may be a bridged structure, a condensed ring structure, or a spiro ring structure.

Examples of the ring in the alicyclic monocyclic hydrocarbon structure include cyclopentane, cyclohexane, cycloheptane, cyclooctane, cyclopentene, cyclohexene, cycloheptene, cyclooctene, and cyclodecene. The alicyclic polycyclic hydrocarbon structure is preferably a bridged alicyclic saturated hydrocarbon structure or a condensed alicyclic saturated hydrocarbon structure. Examples thereof include a bicyclo[2.2.1]heptane structure, a bicyclo[2.2.2]octane structure, a tricyclo[3.3.1.1^{3,7}]decane structure, and a steroid structure.

Examples of the C3 to C20 aliphatic heterocyclic structure include a cyclic ether structure, a lactone structure, a cyclic acetal structure, a cyclic carbonate structure, and a sultone structure. The aliphatic heterocyclic structure may be a monocyclic structure or a polycyclic structure. Also, the polycyclic structure may be may be a bridged structure, a condensed ring structure, or a spiro ring structure. Notably, the C3 to C20 aliphatic heterocyclic structure represented by Y¹ or Y² may be a combined structure of two or more of a bridged structure, a condensed ring structure, and a spiro ring structure. When the C3 to C20 aliphatic heterocyclic structure represented by Y¹ or Y² has a spiro ring structure, the two or more rings forming the spiro ring structure may be a combination of aliphatic heterocycles, or a combination of an aliphatic heterocycle and an alicyclic hydrocarbon ring.

Examples of the ring in the C6 to C20 aromatic ring structure include a benzene ring, a naphthalene ring, an anthracene ring, an indene ring, and a fluorene ring.

When Y¹ or Y² is a monovalent group having a cyclic structure, Y¹ or Y² may include a chain structure in addition to a cyclic structure. When Y¹ or Y² is a group having a chain structure and a cyclic structure, specific examples thereof include a group formed by removing one hydrogen atom from the aforementioned monovalent chain organic group and bonding the aforementioned cyclic structure to the thus-provided divalent group.

From the viewpoint of enhancing hydrophobicity of the resist film obtained from the present composition so as to further increase the difference in solubility in a developer between a light-exposed part and a light-unexposed part, each of Y¹ in the aforementioned formula (2) and Y² in the aforementioned formula (3) is preferably a monovalent group having cyclic structure. Furthermore, when Y¹ or Y² has an iodo group, the monovalent organic group represented by Y¹ or Y² preferably has an aromatic ring structure, from the viewpoint of enhancing the sensitivity of the present composition. When Y¹ or Y² has no iodo group, Y¹ or Y² preferably has an alicyclic hydrocarbon structure or an aliphatic heterocyclic structure, more preferably a bridged alicyclic saturated hydrocarbon structure or a bridged aliphatic heterocyclic structure, from the viewpoint of enhancing transparency of the resist film.

In the compound (B), when the organic anion has an iodo group, at least one species selected from the group consisting of the onium salt represented by the following formula (2A) and the onium salt represented by the following formula (3A) is preferably used as the compound (B). Notably, among them, the onium salt represented by the following formula (2A) is preferably used as an acid-generating agent, and the onium salt represented by the following formula (3A) is preferably used as a light-degradable base.

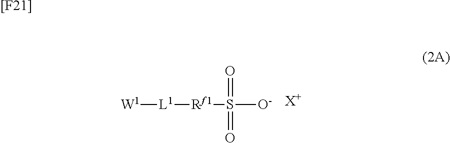

(In the formula (2A), W¹ represents a C5 to C40 monovalent aromatic ring group having an iodo group; L¹ represents a single bond or an (n1+1)-valent organic group; n1 is an integer of 1 or greater; R^{f1} represents an (n1+1)-valent fluorinated hydrocarbon group, when L¹ is a single bond; R^{f1} represents a divalent fluorinated hydrocarbon group, when L¹ is an (n1+1)-valent organic group; and X⁺ represents a monovalent onium cation.)

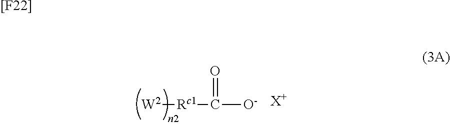

(In the formula (3A), W² represents a C5 to C40 monovalent aromatic ring group having an iodo group; n2 is an integer of 1 or greater; R^{c1} represents a single bond or a divalent organic group, when n2 is 1; R^{c1} represents an (n2+1)-valent organic group, when n2 is 2 or greater; and X⁺ represents a monovalent onium cation.)

In the aforementioned formulas (2A) and (3A), the monovalent aromatic ring group represented by W¹ or W² is preferably a group formed by removing one hydrogen atom from a ring moiety of a substituted aromatic ring. Examples of the aromatic ring include a benzene ring, a naphthalene ring, an anthracene ring, an indene ring, and a fluorene ring. Of these, a benzene ring or a naphthalene ring is preferred, with a benzene ring being more preferred. The substituent substituting a hydrogen atom of the aromatic ring has an iodo group. In addition to an iodo group, the aromatic ring in W¹ or W² may further have a substituent other than an iodo group. Examples of the substituent include a fluoro group, a bromo group, a chloro group, and a hydroxy group. From the viewpoint of sensitivity, W¹ and W² preferably have no fluorine atom.

The divalent fluorinated hydrocarbon group represented by R^{f1} is preferably a linear-chain or branched fluorinated saturated hydrocarbon group. The fluorinated saturated hydrocarbon group preferably has a structure in which any hydrogen atom in a linear-chain alkanediyl group (preferably, a C1 to C5, more preferably a C1 to C3) is substituted by a fluoro group or a fluoroalkyl group. Examples of the fluoroalkyl group include a trifluoromethyl group, a 2,2,2-trifluoroethyl group, a pentafluoroethyl group, a 2,2,3,3,3-pentafluoropropyl group, a 2,2,2-trifluoro-1-(trifluoromethyl)ethyl group, and a 5,5,5-trifluoro-1,1-diethylpentyl group. Of these, a C1 to C3 fluoroalkyl group is preferred, with a trifluoromethyl group being more preferred.

From the viewpoint of enhancing the sensitivity of the present composition, the divalent fluorinated hydrocarbon group represented by R^{f1} preferably has a structure in which a fluorine atom or a trifluoroalkyl group is bound to a carbon atom to which a sulfonate anion (—SO₃—) has been bound. More preferably, a fluorine atom or a trifluoromethyl group is bound.

When R^{f1} is a (n1+1)-valent fluorinated hydrocarbon group, examples include a group formed by removing (n1−1) hydrogen atoms from the aforementioned divalent fluorinated hydrocarbon group.

The parameter n1 is preferably 1 to 5, more preferably 1 to 3, still more preferably 1 or 2.

The divalent linking group represented by L¹ is preferably —O—, —CO—, —COO—, —OCO—, —O—CO—O—, —S—, —SO₂—, —CONH—, —NHCO—, or a divalent group in which any methylene group in a C2 to C10 alkanediyl group is substituted by —O—, —CO—, —COO—, —OCO—, —O—CO—O—, —S—, —SO₂—, —CONH—, or —NHCO—.

Examples of the divalent organic group represented by R^{c1} include a C1 to C20 substituted or unsubstituted alkanediyl group, and a divalent group in which any methylene group in the alkanediyl group is substituted by —O—, —CO—, —COO—, —OCO—, —O—CO—O—, —S—, —SO₂—, —CONH—, or —NHCO—. Examples of the substituent include a fluorine atom and a hydroxy group. The number of present carbons in the divalent organic group represented by R^{c1} is preferably 1 to 10.

When R^{c1} is a (n2+1)-valent organic group, examples include a group formed by removing (n2−1) hydrogen atoms from the aforementioned divalent organic group.

The parameter n2 is preferably 1 to 5, more preferably 1 to 3, still more preferably 1 or 2.

### Cation

The cation included in the compound (B) preferably has a sulfonium cation structure or an iodonium cation structure, from the viewpoints of achieving high sensitivity of the present composition and forming a resist film with higher LWR performance. The same applied to X⁺ in the aforementioned formula (2) or (3). In a particularly preferred mode, the cation included in the compound (B) and X⁺ in the aforementioned formula (2) or (3) each have an aromatic ring bound to a sulfonium cation or an iodonium cation, in which at least one group selected from the group consisting of a fluoroalkyl group, a fluoro group (excepting a fluoro group in the fluoroalkyl group), and an iodo group is bound to the aromatic ring.

From the viewpoint of sensitivity, the cation included in the compound (B) preferably has a triarylsulfonium cation structure or a diaryliodonium cation structure. Specifically, the cation is preferably any of the cations represented by the following formula (2B) or (3B).

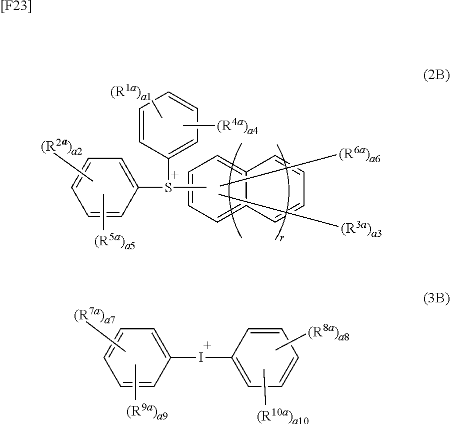

(In the formula (2B), each of R^{1a}, R^{2a}, and R^{3a} independently represents an iodo group, a fluoro group or a fluoroalkyl group; each of R^{4a} and R^{5a} independently represents a monovalent substituent, or R^{4a} and R^{5a} are combined to form a single bond or a divalent group which links the rings to which R^{4a} and R^{5a} are bound; R^{6a} is a monovalent substituent; each of a1, a2, and a3 is independently an integer of 0 to 5, with a1+a2+a3≥1 being satisfied; each of a4, a5, and a6 is independently an integer of 0 to 3; r is 0 or 1, with a1+a4≤5, a2+a5≤5, and a3+a6≤2×r+5 being satisfied; and each of R^{7a} and R^{8a} independently represents an iodo group, a fluoro group or a fluoroalkyl group; each of R^{9a} and R^{10a} independently represents a monovalent substituent; each of a7 and a8 is independently an integer of 0 to 5; and each of a9 and a10 is independently an integer of 0 to 3, with a7+a9≤5 and a8+a10≤5 being satisfied.)

In the aforementioned formulas (2B) and (3B), specific examples including preferred members of fluoroalkyl groups represented by R^{1a}, R^{2a}, R^{3a}, R^{7a}, and R^{8a} include the same groups as mentioned in relation to the fluoroalkyl group included in the divalent fluorinated hydrocarbon group represented by R^{f1} in the aforementioned formula (2A).

Among the aforementioned members, each of R^{1a}, R^{2a}, R^{3a}, R^{7a}, and R^{8a} is preferably an iodo group, a fluoro group, a trifluoromethyl group, a 2,2,2-trifluoroethyl group, or a perfluoroethyl group; more preferably an iodo group, a fluoro group, or a trifluoromethyl group; particularly preferably an iodo group or a fluoro group. Also, a1, a2, and a3 preferably satisfy the condition: “a1+a2+a3≥1”, and a7 and a8 preferably satisfy the condition: “a7+a8≥1”. By use of an onium salt having a structure in which an iodo group, a fluoro group, or a trifluoromethyl group is directly bound to an aromatic ring present in a triarylsulfonium cation structure or a diaryliodonium cation structure, the sensitivity of the present composition can be further enhanced, and a composition exhibiting excellent LWR performance can be provided.

In the aforementioned formulas (2B) and (3B), specific examples of the monovalent substituents represented by R^{4a}, R^{5a}, R^{6a}, R^{9a}, and R^{10a} include a chloro group, a bromo group, a substituted or unsubstituted alkyl group (excepting a fluoroalkyl group), a substituted or unsubstituted alkoxy group, a substituted or unsubstituted cycloalkyl group, a substituted or unsubstituted cycloalkyloxy group, an ester group, an alkylsulfonyl group, a cycloalkylsulfonyl group, a hydroxy group, a carboxy group, a cyano group, and a nitro group.

When one or more parameters (a1, a2, and a3) showing the numbers of R^{1a}, R^{2a}, and R^{3a}, respectively, in the aforementioned formula (2B) are integers of 1 or greater, and each of the groups (R^{1a}, R^{2a}, and R^{3a}) corresponding to the parameters is an iodo group, the cation having the structure is a “cation having an iodo group”. Similarly, when one or more parameters (a7 and a8) showing the numbers of R^{7a} and R^{8a}, respectively, in the aforementioned formula (3B) are integers of 1 or greater, and each of the groups (R^{7a} and R^{8a}) corresponding to the parameters is an iodo group, the cation having the structure is a “cation having an iodo group”.

When the cation forming the compound (B) has an iodo group, the organic anion forming the compound (B) may have no iodo group. In this case, no particular limitation is imposed on the structure of the organic anion. Specific examples of the organic anion when the cation forming the compound (B) has an iodo group and the organic anion has no iodo group include the following anions.

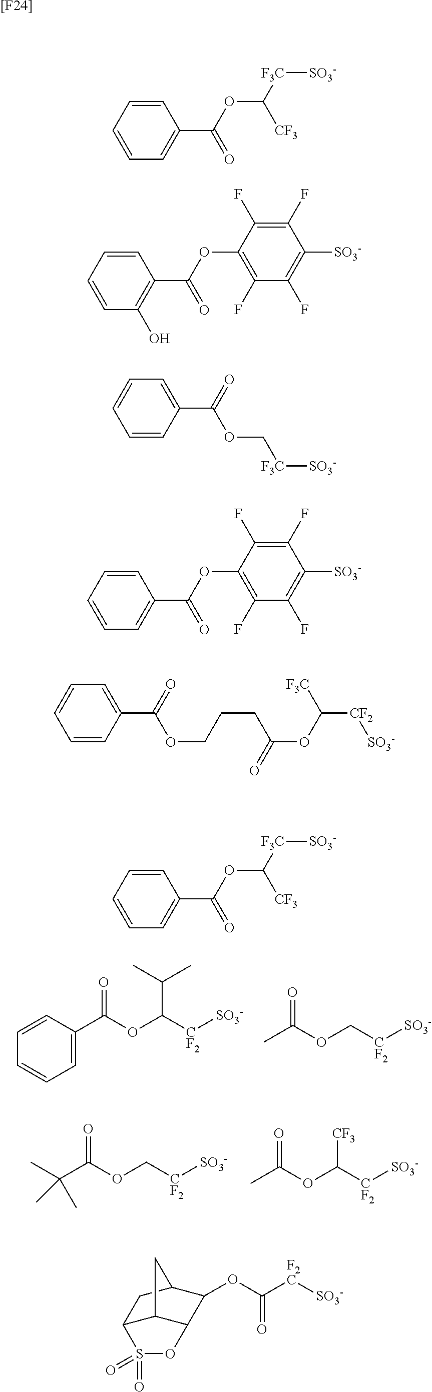

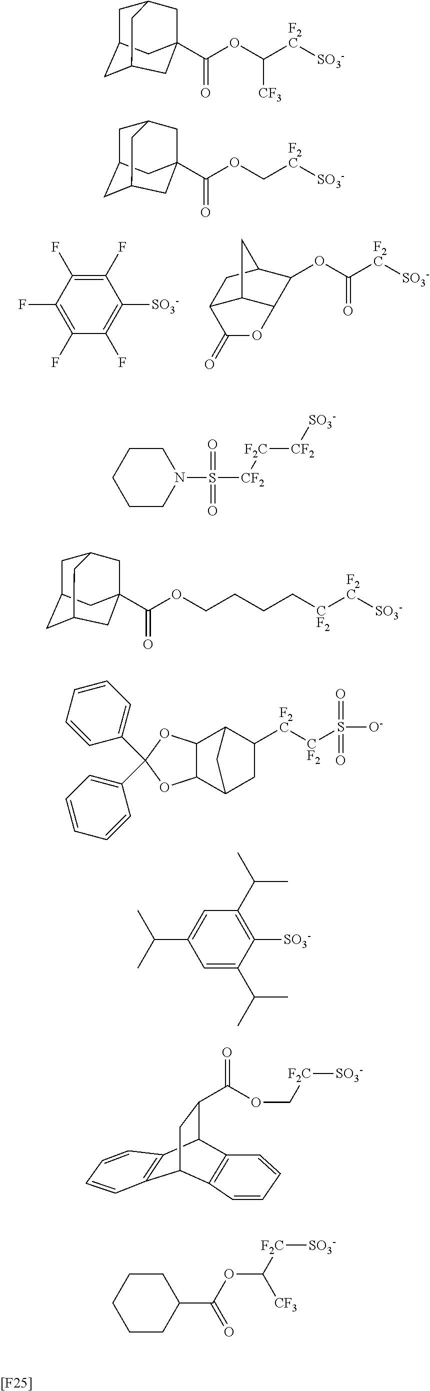

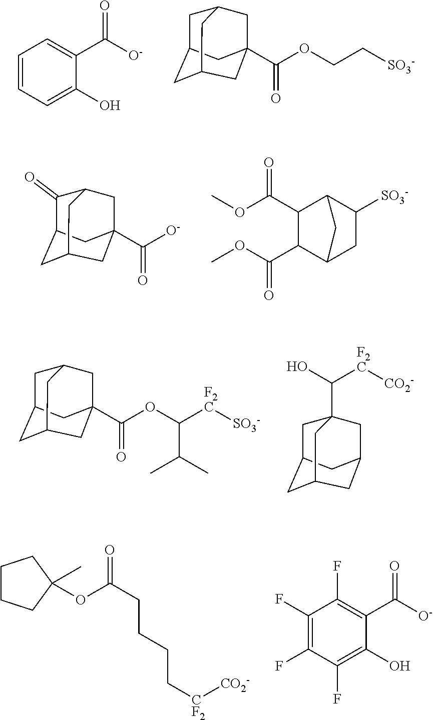

Specific examples of the onium salt suitably used as the acid-generating agent (B¹) include the compounds represented by the following structures. However, the compound (B) and the acid-generating agent (B¹) are not limited to the following examples.

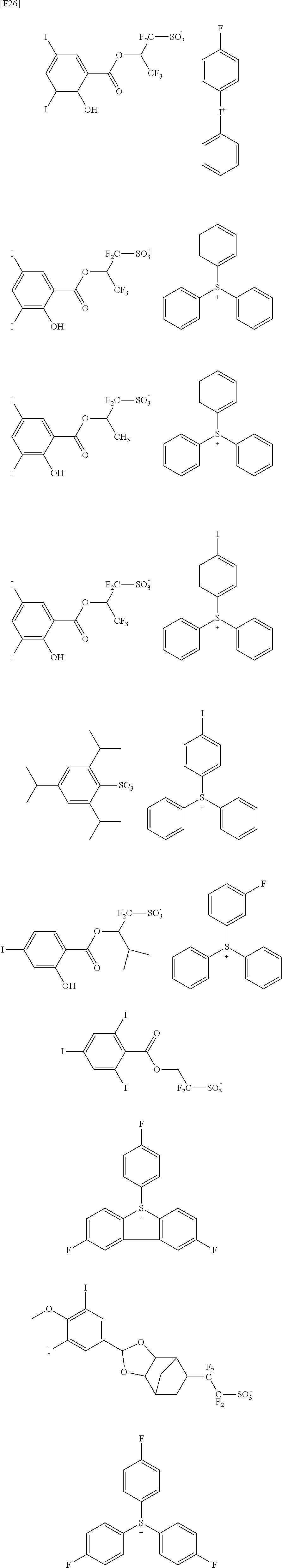

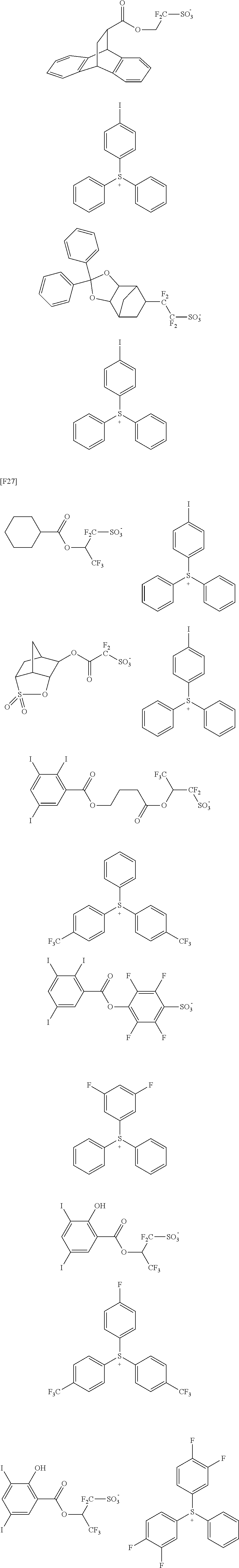

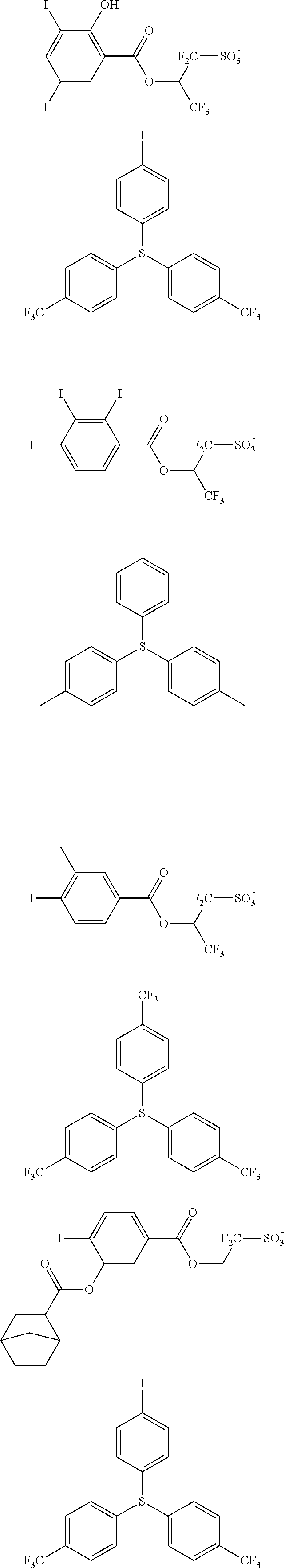

Specific examples of the onium salt suitably used as the light-degradable base (B2) include the compounds represented by the following structures. However, the compound (B) and the light-degradable base (B2) are not limited to the following examples.

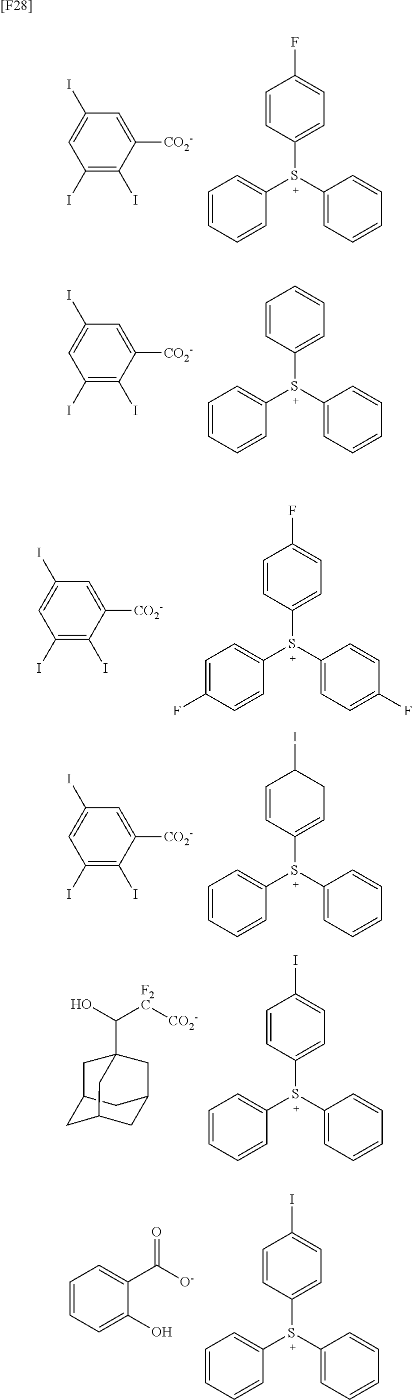

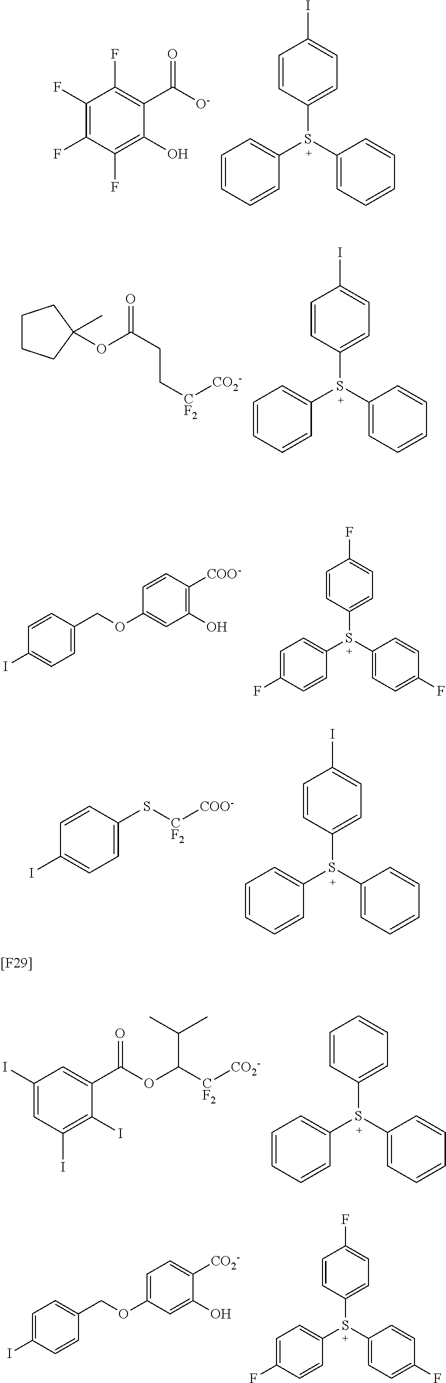

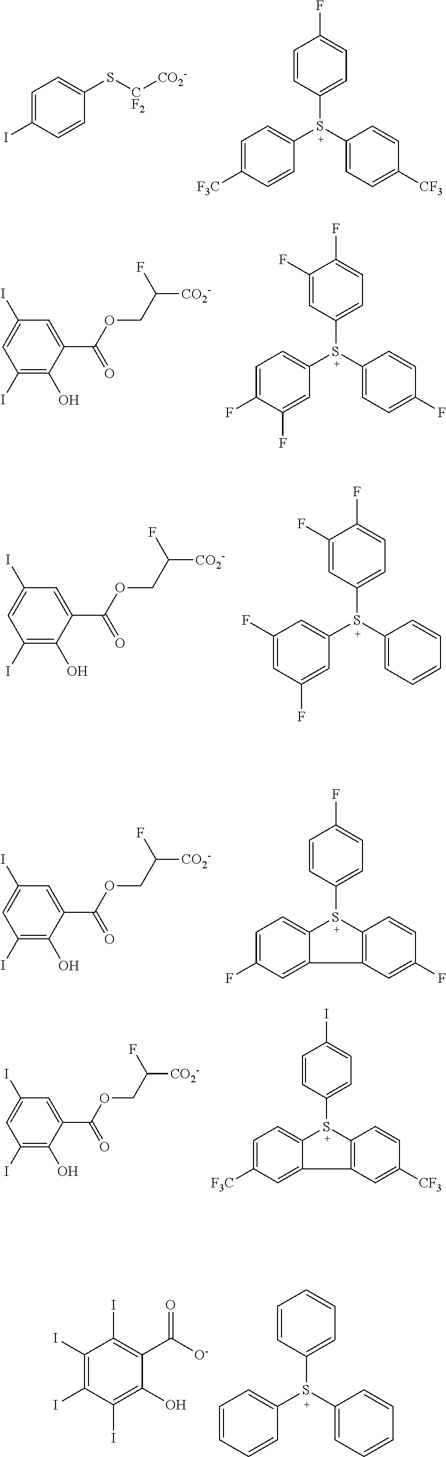

The relative amount of the compound (B) in the present composition, with respect to 100 parts by mass of the base resin contained in the present composition, is preferably 0.5 parts by mass or more, more preferably 2 parts by mass or more, still more preferably 5 parts by mass or more. Also, the relative amount of the compound (B), with respect to 100 parts by mass of the base resin, is preferably 65 parts by mass or less, more preferably 50 parts by mass or less, still more preferably 40 parts by mass or less. By adjusting the compound (B) content to satisfy the aforementioned conditions, the effect of enhancing the sensitivity and LWR performance of the present composition can be achieved, while occurrence of development failure is satisfactorily suppressed.

The compound (B) will be described in more detail. When the present composition contains the acid-generating agent (B1) as the compound (B), the relative amount of the acid-generating agent (B1) in the present composition, with respect to 100 parts by mass of the base resin contained in the present composition, is preferably 1 part by mass or more, more preferably 5 parts by mass or more, still more preferably 10 parts by mass or more. Also, the relative amount of the acid-generating agent (B1), with respect to 100 parts by mass of the base resin, is preferably 50 parts by mass or less, more preferably 40 parts by mass or less, still more preferably 30 parts by mass or less. By adjusting the acid-generating agent (B1) content to satisfy the aforementioned conditions, the effect of enhancing the sensitivity and LWR performance of the present composition can be achieved. The acid-generating agent (B1) may be used singly or in combination of two or more species.

When the present composition contains the light-degradable base (B2) as the compound (B), the relative amount of the light-degradable base (B2) in the present composition, with respect to 100 parts by mass of the base resin contained in the present composition, is preferably 1 part by mass or more, more preferably 3 parts by mass or more, still more preferably 5 parts by mass or more. Also, the relative amount of the light-degradable base (B2), with respect to 100 parts by mass of the base resin, is preferably 30 parts by mass or less, more preferably 25 parts by mass or less, still more preferably 20 parts by mass or less. By adjusting the light-degradable base (B2) content to satisfy the aforementioned conditions, the effect of enhancing the sensitivity and LWR performance of the present composition can be achieved. The light-degradable base (B2) may be used singly or in combination of two or more species.

There has been elucidated no conceivable reason why the sensitivity and LWR performance of the radiation-sensitive composition can be enhanced and, further, occurrence of development failure can be suppressed, by virtue of the present composition containing the polymer (A) and the compound (B). However, the following reason would be conceived. A study conducted by the present inventors previously revealed that, although a radiation-sensitive acid-generator having an iodo group exhibits high sensitivity to radiation, the presence of the iodine atom tends to result in high hydrophobicity. Therefore, when such a radiation-sensitive acid-generator having an iodo group (i.e., the compound (B)) is used, conceivably, solubility in a developer decreases, thereby evoking generation of development failure, although high sensitivity may be achieved. In contrast, by incorporating a polymer including a structural unit represented by the aforementioned formula (1) (i.e., the polymer (A)) into a radiation-sensitive composition, conceivably, the solubility of the radiation-sensitive composition in a developer increases, whereby excellent LWR performance and reduction of development failure can be achieved, while the sensitivity of the radiation-sensitive composition is enhanced.

### <Additional Component>

The present composition may further contain a component differing from the polymer (A) and the compound (B). Examples of such an additional component which the present composition may contain include a polymer which does not include a structural unit represented by the aforementioned formula (1) (hereinafter may also be referred to as an “additional polymer”), a radiation-sensitive acid-generating agent differing from the acid-generating agent (B1) (hereinafter may also be referred to as an “additional acid-generating agent”), an acid diffusion control agent differing from the light-degradable base (B2) (hereinafter may also be referred to as an “additional acid diffusion control agent”), and a solvent.

### (Additional Polymer)

When the polymer (A) is a high-fluorine content polymer, the present composition preferably contains a base resin as another component other than the polymer (A). The base resin serving as the additional polymer is a polymer differing from the polymer (A) and having a fluorine atom content (by mass) lower than that of the polymer (A). The base resin serving as the additional polymer is preferably a polymer including a structural unit having an acid-dissociable group (i.e., a third structural unit) (hereinafter may also be referred to as a “polymer (C)”). Specific examples including preferred members of the third structural unit in the polymer (C) are the same structural units as described in relation to the third structural unit which the polymer (A) may include.

The third structural unit content of the polymer (C) is preferably greater than the third structural unit content of the polymer (A), from the viewpoints of providing a sufficiently large difference in solubility in a developer between the light-exposed part and the light-unexposed part so as to yield a resist film having excellent LWR performance and sufficiently reducing occurrence of development failure. More specifically, the third structural unit content of the polymer (C), with respect to all the structural units forming the polymer (C), is preferably 10 mol % or more, more preferably 20 mol % or less, still more preferably 30 mol % or more. Also, the third structural unit content, with respect to all the structural units forming the polymer (C), is preferably 90 mol % or more, more preferably 85 mol % or less, still more preferably 80 mol % or less. Notably, the polymer (C) may include only one third structural unit or two or more members thereof.

In addition, the polymer (C) may further include a structural unit differing from the third and first structural units, in addition to the third structural unit. Examples of such a structural unit include the same structural unit as exemplified in relation to the additional structural units which the polymer (A) may include (e.g., second, fourth, and fifth structural units).

Particularly when the present composition is used in pattern formation employing light exposure by an electron beam or a radiation having a wavelength of 50 nm or shorter (e.g., EUV), preferably, the polymer (C) further includes the aforementioned fourth structural unit. When the polymer (C) includes a fourth structural unit, the relative amount of the fourth structural unit in the polymer (C), with respect to all the structural units forming the polymer (C), is preferably 10 mol % or more, more preferably 15 mol % or more, still more preferably 20 mol % or more. Also, the relative amount of the fourth structural unit in the polymer (C), with respect to all the structural units forming the polymer (C), is preferably 80 mol % or less, more preferably 75 mol % or less. Notably, the polymer (C) may include the fourth structural unit singly or in combination of two or more members.

Meanwhile, when the polymer (A) contained in the present composition forms a base resin, the present composition may further contain a high-fluorine content polymer as an additional polymer. Examples of the additional polymer serving as the high-fluorine content polymer include a polymer including a second structural unit, as exemplified in relation to the description of the polymer (A). Also, the high-fluorine content polymer serving as the additional polymer may further include, in addition to the second structural unit, one or more members of the third to fifth structural units, a structural unit having a non-acid-dissociable hydrocarbon group, etc.

### (Additional Acid-Generating Agent)

As the additional acid-generating agent, an onium salt which is formed of a cation and an organic anion and which has no iodo group may be suitably used. Specific examples of the additional acid-generating agent include an onium salt formed of an organic anion having no iodo group and a cation having no iodo group, which is exemplified in relation to the description of the compound (B).

Alternatively, as the additional acid-generating agent, there may be used a polymer formed of a structural unit having a partial structure that can generate an acid in the present composition through exposure to light (e.g., a structural unit having a partial structure formed of a triarylsulfonium cation and an organic anion or a structural unit having a partial structure formed of a diaryliodonium cation and an organic anion).

The relative amount of the additional acid-generating agent in the present composition; i.e., the total amount of the acid-generating agent (B1) and the additional acid-generating agent, with respect to 100 parts by mass of the base resin contained in the present composition, is preferably 1 part by mass or more, more preferably 2 parts by mass or more, still more preferably 3 parts by mass or more. Also, the relative amount of the additional acid-generating agent content; i.e., the total amount of the acid-generating agent (B1) and the additional acid-generating agent, with respect to 100 parts by mass of the base resin contained in the present composition, is preferably 50 parts by mass or less, more preferably 40 parts by mass or less, still more preferably 30 parts by mass or less. The additional acid-generating agent may be used singly or in combination of two or more species.

### (Additional Acid Diffusion Control Agent)

As the additional acid diffusion control agent, an onium salt which is formed of a cation and an organic anion and which has no iodo group is preferably used. Specific examples of the onium salt include an onium salt formed of an organic anion having no iodo group and a cation having no iodo group, which is exemplified in relation to the description of the compound (B).

Also, as the additional acid diffusion control agent, there may be used a compound other than the light-degradable base such as an amino group-containing compound (e.g., an alkylamine, an aromatic amine, or a polyamine), an amido group-containing compound, a urea compound, a nitrogen-containing heterocyclic compound, or a nitrogen-containing compound having an acid-dissociable group).

The relative amount of the additional acid diffusion control agent in the present composition; i.e., the total amount of the acid diffusion control agent (B2) and the additional acid diffusion control agent, with respect to 100 parts by mass of the base resin contained in the present composition, is preferably 1 part by mass or more, more preferably 3 parts by mass or more, still more preferably 5 parts by mass or more. Also, the relative amount of the additional acid diffusion control agent; i.e., the total amount of the acid diffusion control agent (B2) and the additional acid diffusion control agent, with respect to 100 parts by mass of the base resin contained in the present composition, is preferably 30 parts by mass or less, more preferably 25 parts by mass or less, still more preferably 20 parts by mass or less. The additional acid diffusion control agent may be used singly or in combination of two or more species.

When the present composition contains an acid-generating agent and an acid diffusion control agent, the relative amount of the acid diffusion control agent, with respect to the amount of the acid-generating agent contained in the present composition (the total amount thereof in the case two or more species are used), is preferably 1 mol % or more, more preferably 2 mol % or more, still more preferably 5 mol % or more. Also, the relative amount of the acid diffusion control agent, with respect to the amount of the acid-generating agent contained in the present composition, is preferably 50 mol % or less, more preferably 40 mol % or less. By adjusting the acid diffusion control agent content to satisfy the above conditions, LWR performance of the present composition can be further enhanced.

### <Solvent>

No particular limitation is imposed on the solvent, so long as the solvent can dissolve or disperse the components incorporated into the present composition therein. Examples of the solvent include an alcohol, an ether, a ketone, an amide, an ester, and a hydrocarbon.

Examples of the alcohol include C1 to C18 aliphatic monoalcohols such as 4-methyl-2-pentanol and n-hexanol; C3 to C18 alicyclic monoalcohols such as cyclohexanol; C2 to C18 polyhydric alcohols such as 1,2-propylene glycol; and C3 to C19 polyhydric alcohol partial ethers such as propylene glycol monomethyl ether. Examples of the ether include dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic ring-containing ethers such as diphenyl ether and anisole.

Examples of the ketone include chain ketones such as acetone, methyl ethyl ketone, methyl n-propyl ketone, methyl n-butyl ketone, diethyl ketone, methyl isobutyl ketone, 2-heptanone, ethyl n-butyl ketone, methyl n-hexyl ketone, diisobutyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amide include cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the ester include monocarboxylic acid ester-type solvents such as n-butyl acetate and ethyl lactate; polyhydric alcohol carboxylates such as propylene glycol acetate; polyhydric alcohol partial ester carboxylates such as propylene glycol monomethyl ether acetate; polybasic carboxylic acid diesters such as diethyl oxalate; carbonates such as dimethyl carbonate and diethyl carbonate; and cyclic esters such as γ-butyrolactone. Examples of the hydrocarbon include C5 to C12 aliphatic hydrocarbons such as n-pentane and n-hexane; and C6 to C16 aromatic hydrocarbons such as toluene and xylene.

Among the above-exemplified solvents, the solvent preferably includes at least one member selected from the group consisting of the ester and the ketone, more preferably at least one member selected from the group consisting of polyhydric alcohol partial ether carboxylates and cyclic ketones, still more preferably one or more species from among propylene glycol monomethyl ether acetate, ethyl lactate, and cyclohexanone. These solvents may be used singly or in combination of two or more species.

### <Additional and Optional Component>

The present composition may further contain a component which differs from the aforementioned polymer (A), compound (B), polymer (C), additional acid-generating agent, additional acid diffusion control agent, and solvent (hereinafter may also be referred to as an “additional and optional component”). Examples of the additional and optional component include a surfactant, a compound having an alicyclic skeleton (e.g., 1-adamantanecarboxylic acid, 2-adamantanone, or t-butyl deoxycholate), a sensitizer, and a localization accelerator. So long as the effects of the present disclosure are not impaired, the additional and optional component content of the present composition may be appropriately set depending on the property of the component.

### <Method of Producing Radiation-Sensitive Composition>

The present composition may be produced through, for example, the following procedure: mixing the polymer (A) and the compound (B) with optional components such as the polymer (C) and the solvent at desired proportions and filtering the resultant mixture by means of a filter (e.g., a filter having a pore size of about 0.2 m) or the like. The solid content of the present composition is preferably 0.1 mass % or more, more preferably 0.5 mass % or more, still more preferably 1 mass % or more. Also, the solid content of the present composition is preferably 50 mass % or less, more preferably 20 mass % or less, still more preferably 5 mass % or less. By adjusting the solid content of the present composition to satisfy the above conditions, coatability of the composition can be enhanced, to thereby obtain a resist pattern having a suitable shape, which is preferred.

The thus-obtained present composition may also be used as a composition for forming a positive pattern, which is employed for pattern formation by use of an alkaline developer. Alternatively, the present composition may be used as a composition for forming a negative pattern, which is employed for pattern formation by use of a developer containing organic solvent.

### <Method for Forming Resist Pattern>

The method of forming a resist pattern of the present disclosure includes a step of applying the present composition onto one surface of a substrate (hereinafter may also be referred to as a “application step”), a step of exposing to light a resist film obtained in the application step (hereinafter may also be referred to as a “light-exposure step”), and a step of developing the light-exposed resist film (hereinafter may also be referred to as a “development step”). Examples of the pattern obtained through the method of forming a resist pattern of the present disclosure include a line-and-space pattern and a hole pattern. Since a resist film is formed by use of the present composition in the method of forming a resist pattern of the present disclosure, suitable sensitivity and lithographic characteristics are achieved, and a resist pattern which has few development failure can be formed. The steps will next be described in detail.

### [Application Step]

In the application step, the present composition is applied onto one surface of a substrate, to thereby form a resist film on the substrate. A conventionally known substrate can be used as a substrate on which resist film is to be formed. Examples of the substrate include a silicon wafer and a wafer coated with silicon dioxide or aluminum. Alternatively, an organic or inorganic anti-reflection film (see, for example, Japanese Patent Publication (kokoku) No. 1994-12452) may be formed on a substrate to be used. Examples of the method of applying the present composition include spin coating, flow casting, and roller coating. After application, the applied composition may be subjected to pre-baking (PB, also called soft baking (SB)) so as to evaporate the solvent remaining in the coating film. The temperature of PB is preferably 60 to 140° C., more preferably 80 to 130° C. The time of PB is preferably 5 to 600 seconds, more preferably 10 to 300 seconds. The average thickness of the formed resist film is preferably 10 to 1,000 nm, more preferably 20 to 500 nm.

### [Light-Exposure Step]

In the light-exposure step, the resist film formed through the above application step is exposed to light. In the light exposure, the resist film is irradiated with radiation by the mediation of a photomask or, in some cases, a liquid immersion medium such as water. The radiation is selected in accordance with the line width of a target pattern, and examples thereof include electromagnetic waves such as visible light, a UV ray, a far-UV ray, an extreme UV (EUV) ray, an X-ray, and a γ-ray; and charged particle rays such as an electron beam and an α-ray. Among them, the radiation applied to the resist film formed from the present composition is preferably a far-UV ray, an EUV ray, or an electron beam, more preferably ArF excimer laser light (wavelength: 193 nm), KrF excimer laser light (wavelength: 248 nm), an EUV ray, or an electron beam, still more preferably ArF excimer laser light, an EUV ray, or an electron beam.

After completion of the above light exposure, post exposure baking (PEB) is preferably performed, so as to promote dissociation of an acid-dissociable group by the mediation of an acid generated through light exposure by an acid-generating agent in the light-exposed part of the resist film. Through PEB, the difference in dissolution performance with respect to a developer between the light-exposed part and the light-unexposed part can be increased. The temperature of PEB is preferably 50 to 180° C., more preferably 80 to 130° C. The time of PEB is preferably 5 to 600 seconds, more preferably 10 to 300 seconds.

### [Development Step]

In the development step, the resist film which has been exposed to light in the above step is developed, whereby a resist pattern of interest can be formed. The developer may be an alkaline developer or an organic solvent developer. The developer may be appropriately chosen in accordance with the target type of the pattern (i.e., a positive-type pattern or a negative-type pattern).

Examples of the developer employed in the alkali development include aqueous alkaline solutions in which at least one species from among alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo[5.4.0]-7-undecene, 1,5-diazabicyclo[4.3.0]-5-nonene, and the like is dissolved. Among such alkaline solutions, an aqueous TMAH solution is preferred. In the case of development with an organic solvent, examples of the developer include organic solvents such as hydrocarbons, ethers, esters, ketones, and alcohols; and a solvent containing any of the above organic solvents. Examples of the organic solvent include one or more solvents as exemplified in relation to the solvent which may be added to the present composition. No particular limitation is imposed on the development method, and development may be conducted through an appropriately selected known method.

The present composition described hereinabove, containing the polymer (A) and the compound (B), exhibits high sensitivity to the exposure light and can provide a resist pattern which exhibits excellent LWR performance and few development failures. Thus, the present composition can be suitably employed for forming a fine resist pattern in a lithography step in production of electronic devices such as semiconductor devices and liquid crystal devices.

According to the present disclosure described in detail hereinabove, the following means are provided.
[Means 1]A radiation-sensitive composition comprising (A) a polymer including a structural unit represented by the aforementioned formula (1) and (B) an onium salt compound which is formed of an organic anion and a cation, in which the organic anion or the cation or both have an iodo group, and which generates an acid through exposure to radiation.
[Means 2] The radiation-sensitive composition as described in [Means 1], wherein the compound (B) is represented by the aforementioned formula (2) or (3).
[Means 3] The radiation-sensitive composition as described in [Means 1] or [Means 2], wherein the compound (B) has a structure in which an iodo group is bound to an aromatic ring.
[Means 4] The radiation-sensitive composition as described in any of [Means 1] to [Means 3], which contains a compound represented by the aforementioned formula (2A) as the compound (B).
[Means 5] The radiation-sensitive composition as described in any of [Means 1] to [Means 4], which contains a compound represented by the aforementioned formula (3A) as the compound (B).
[Means 6] The radiation-sensitive composition as described in any of [Means 1] to [Means 5], which further contains an onium salt compound which can generate an acid having an acidity lower than that of an acid generated by the compound (B) and which differs from the compound (B).
[Means 7] The radiation-sensitive composition as described in any of [Means 1] to [Means 6], which further contains an onium salt compound which can generate an acid having an acidity higher than that of an acid generated by the compound (B) and which differs from the compound (B).
[Means 8] The radiation-sensitive composition as described in any of [Means 1] to [Means 5], which contains, as the compound (B), a first onium salt compound, and a second onium salt compound which can generate an acid having an acidity lower than that of an acid generated by the first onium salt compound.
[Means 9] The radiation-sensitive composition as described in any of [Means 1] to [Means 8], wherein the cation has a sulfonium cation structure or an iodonium cation structure.
[Means 10] The radiation-sensitive composition as described in [Means 9], wherein the cation includes an aromatic ring bound to a sulfonium cation or an iodonium cation, and at least one group selected from the group consisting of a fluoroalkyl group, a fluoro group (excepting a fluoro group in the fluoroalkyl group), and an iodo group is bound to the aromatic ring.
[Means 11] The radiation-sensitive composition as described in any of [Means 1] to [Means 10], wherein the polymer (A) further includes a structural unit having an acid-dissociable group.
[Means 12] The radiation-sensitive composition as described in any of [Means 1] to [Means 11], wherein the polymer (A) further includes a structural unit having a hydroxy group bound to an aromatic ring.
[Means 13] The radiation-sensitive composition as described in any of [Means 1] to [Means 12], wherein the polymer (A) further includes a structural unit having a fluorine atom.
[Means 14] The radiation-sensitive composition as described in [Means 13], wherein the polymer (A) includes the structural unit having a fluorine atom in a relative amount, with respect to all the structural units forming the polymer (A), of 45 to 99 mol %.
[Means 15] The radiation-sensitive composition as described in any of [Means 1] to [Means 14], which contains the polymer (A) in a relative amount, with respect to the entire amount of the composition excepting the solvent, of 0.1 to 20 mass %.
[Means 16] The radiation-sensitive composition as described in any of [Means 1] to [Means 15], wherein the polymer (A) includes the structural unit represented by the aforementioned formula (1) in a relative amount, with respect to all the structural units forming the polymer (A), of 1 to 55 mol %.
[Means 17] The radiation-sensitive composition as described in any of [Means 1] to [Means 16], wherein the composition further contains a polymer which includes a structural unit having an acid-dissociable group (C) and which differs from the polymer (A), and the polymer (A) has a fluorine atom content by mass higher than that of the polymer (C).
[Means 18] The radiation-sensitive composition as described in any of [Means 1] to [Means 17], wherein E⁺ in the aforementioned formula (1) is represented by the aforementioned formula (e-1), (e-2), or (e-3).
[Means 19] The radiation-sensitive composition as described in any of [Means 1] to [Means 18], wherein D⁻ in the aforementioned formula (1) is any of —COO⁻, —SO₃⁻, —PO₃⁻, —POO⁻, or —O⁻.
[Means 20]A method for forming a resist pattern, including a step of forming a resist film on a substrate by applying a radiation-sensitive composition as recited in any of [Means 1] to [Means 19] onto a substrate, a step of exposing the resist film to light, and a step of developing the light-exposed resist film.

### EXAMPLES

The present disclosure will next be described in detail by way of examples, which should not be construed as limiting the disclosure thereto.

### <Determination of Physical Properties of Polymer>

### [Determination of Weight Average Molecular Weight (Mw), Number Average Molecular Weight (Mn), and Molecular Weight Distribution (Mw/Mn)]

Determined through gel permeation chromatography (GPC) with GPC columns (G2000HXL×2, G3000HXL×1, and G4000HXL×1) (products of Tosoh Corp.) under the following conditions; i.e., flow rate: 1.0 mL/min, eluent: tetrahydrofuran, column temperature: 40° C., and standard: monodispersed polystyrene.

[¹H-NMR and ¹³C-NMR Analysis]

Performed by means of a nuclear magnetic resonance apparatus (“JNM-Delta400”, product of JEOL).

### <Synthesis of Polymers>

Monomers used for synthesizing polymers in the Examples and Comparative Examples are as follows. Notably, in the following Synthesis Examples, unless otherwise specified, the unit “parts by mass” is based on the total mass of the monomers used as 100 parts by mass. The unit “mol %” is based on the total amount by mole of the monomers used as 100 mol %.

### (Monomers for Providing Structural Units Represented by Formula (1) (i.e., First Structural Units))

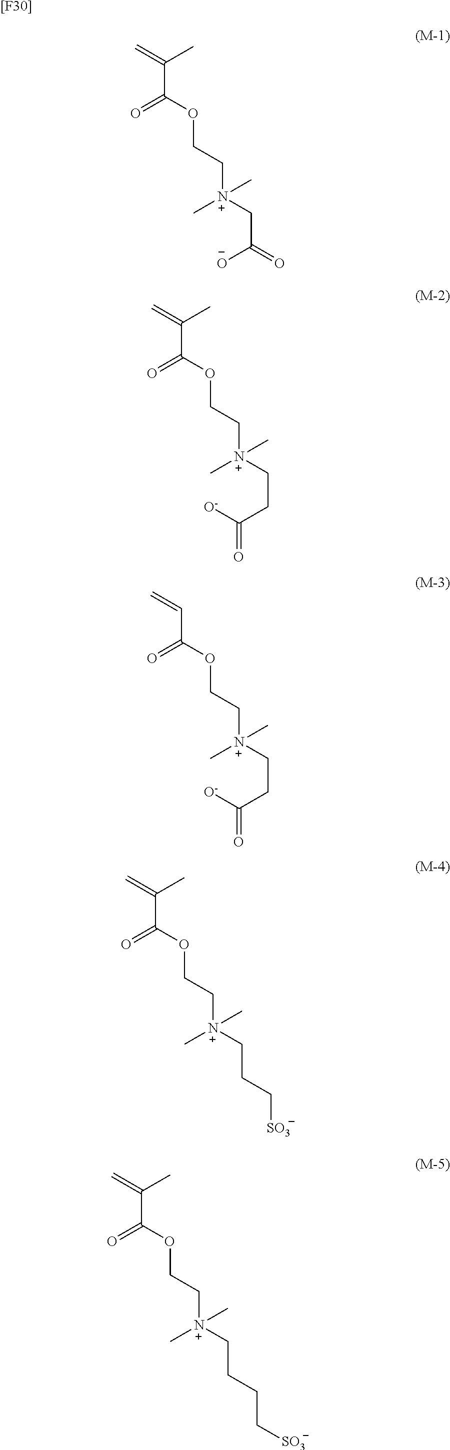

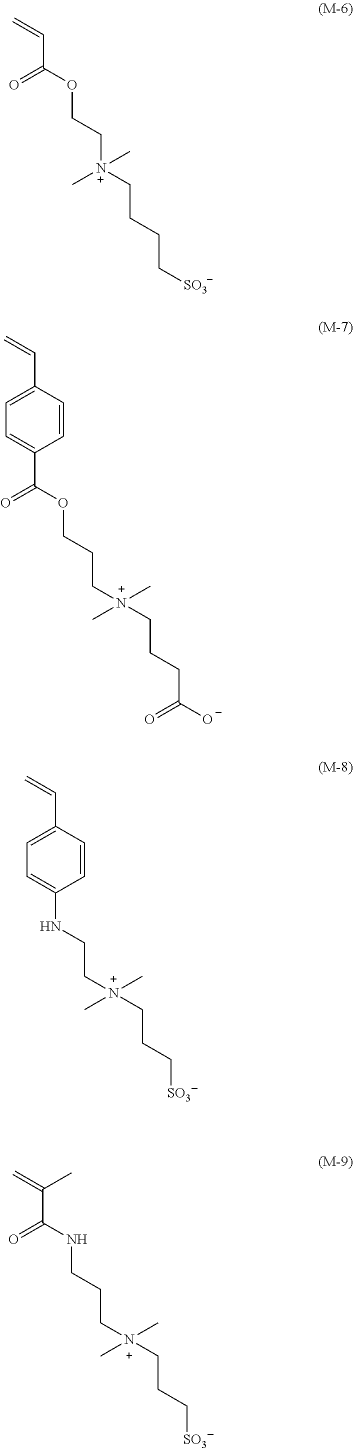

### (Monomers for Providing Additional Structural Units)

### Second Structural Units

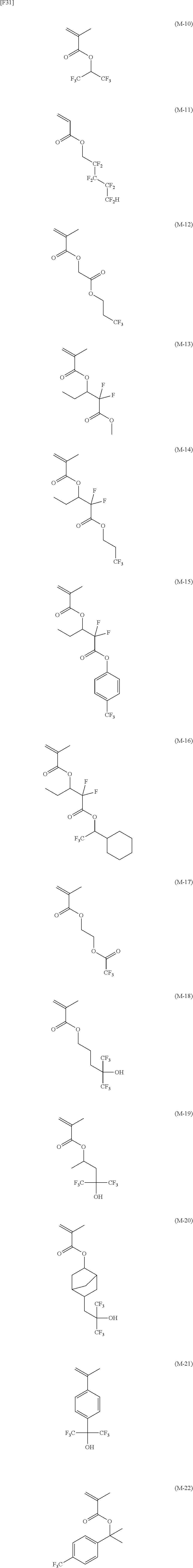

Units Other than Second Structural Unit

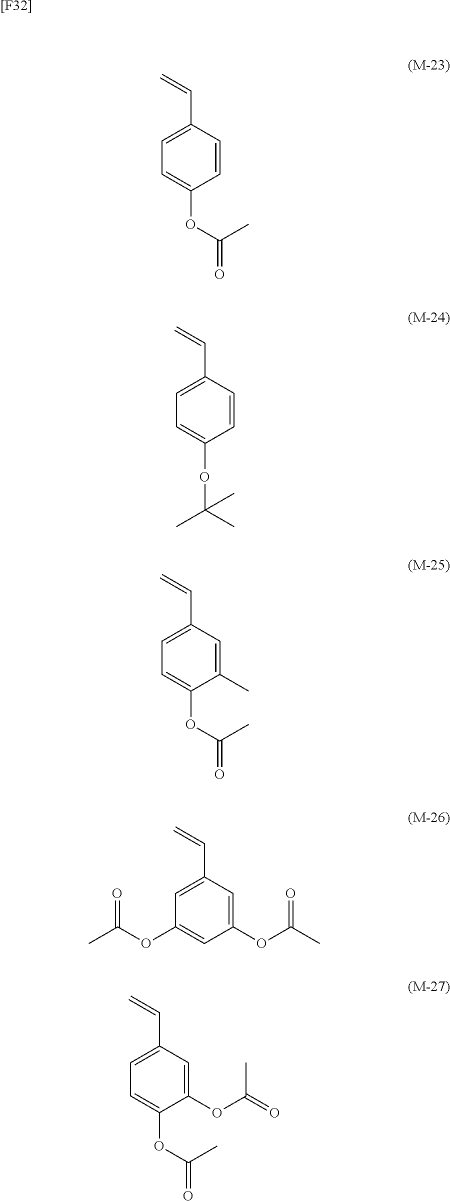

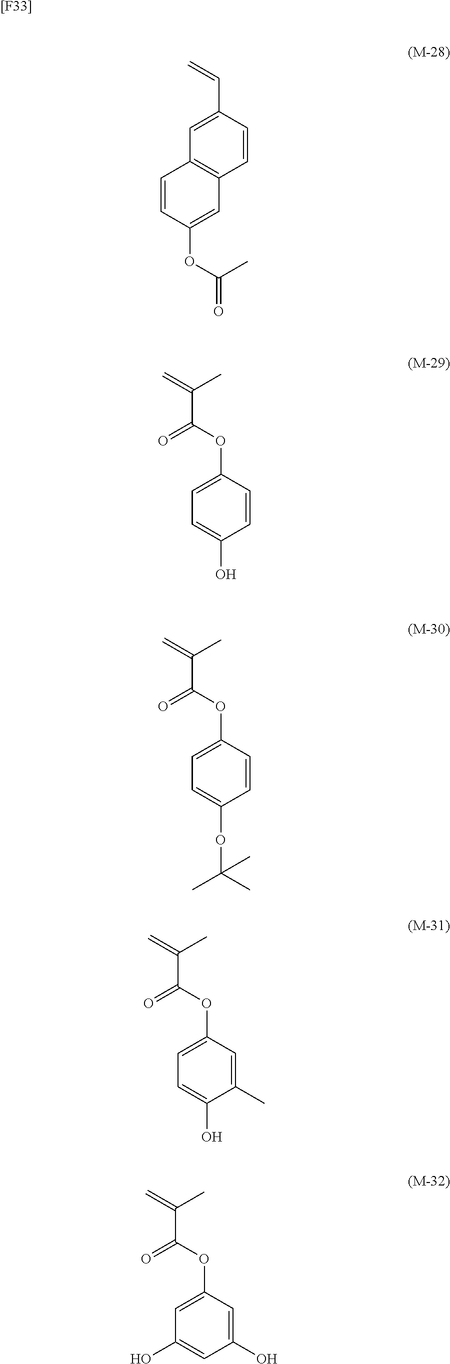

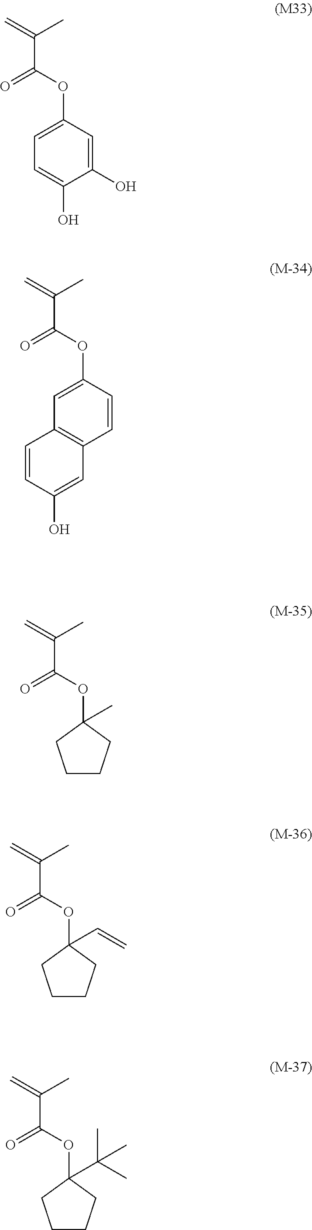

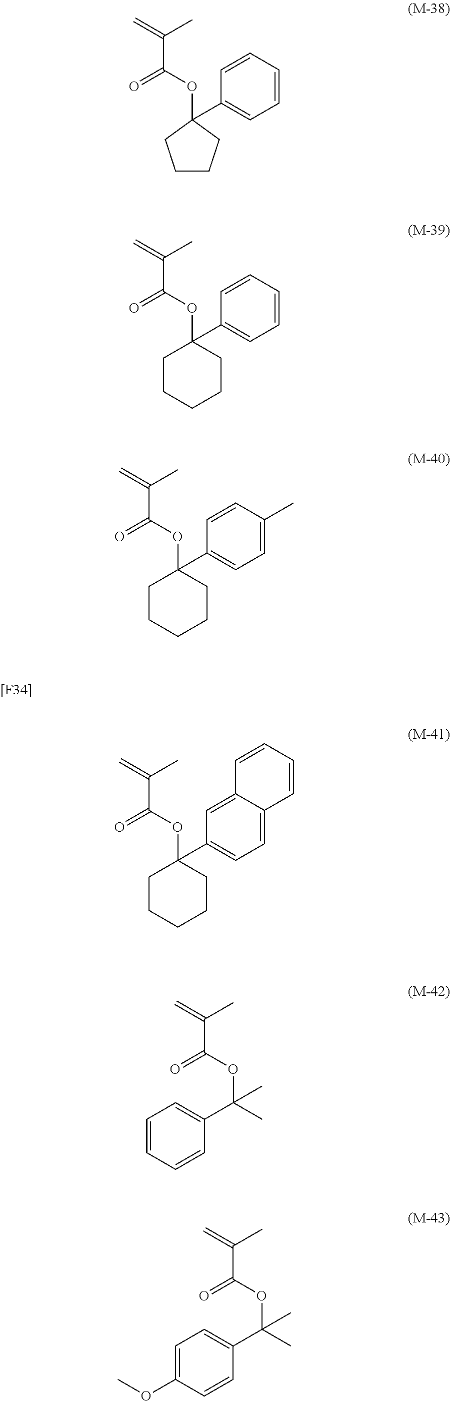

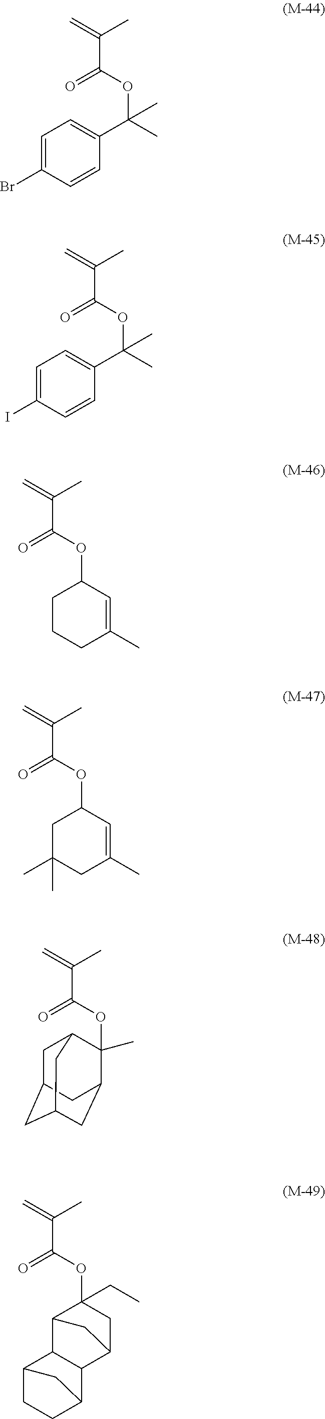

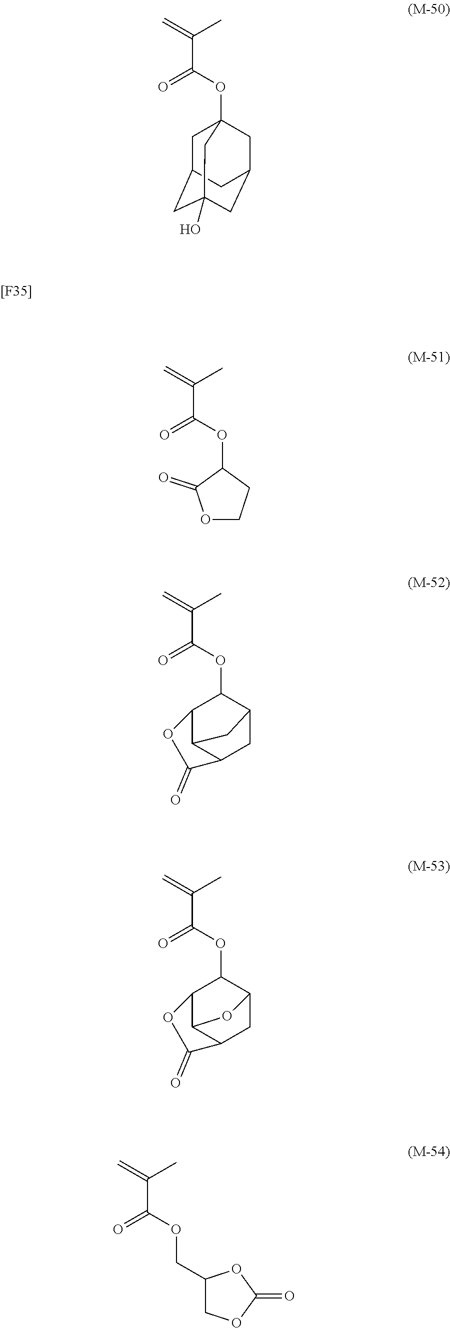

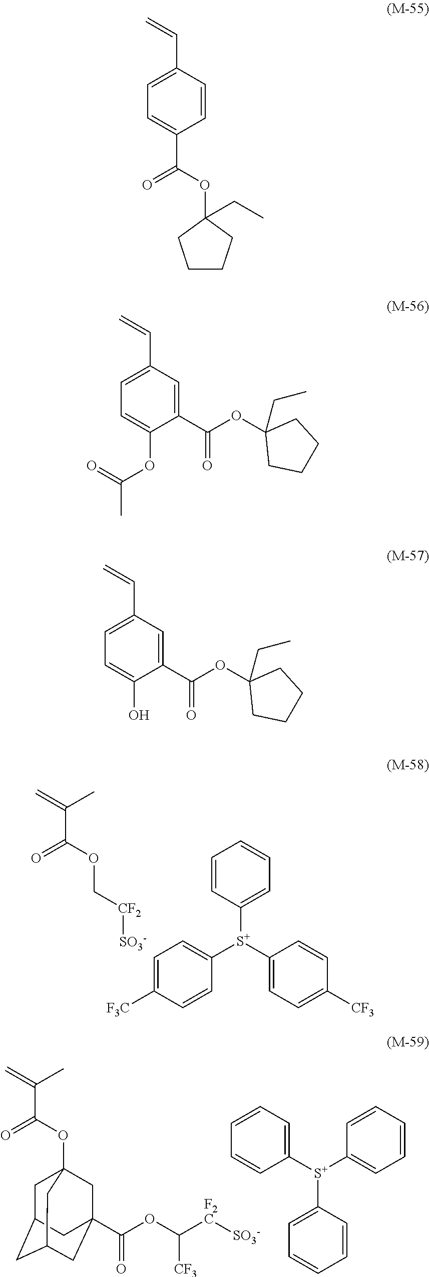

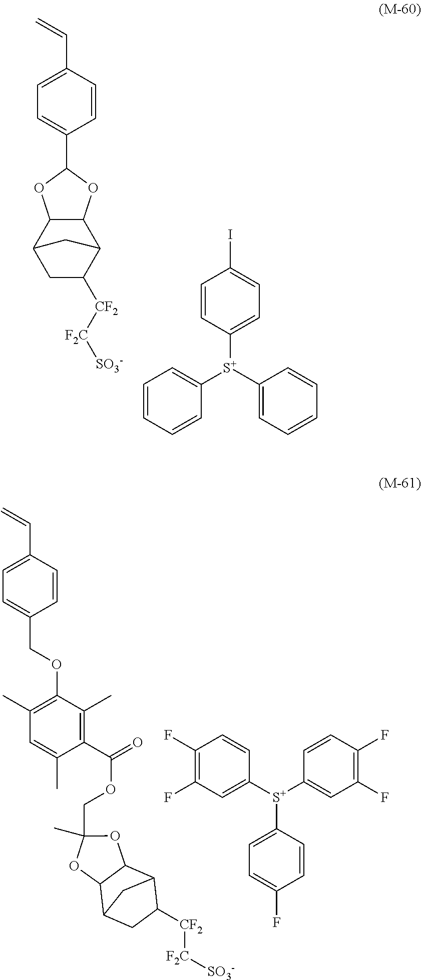

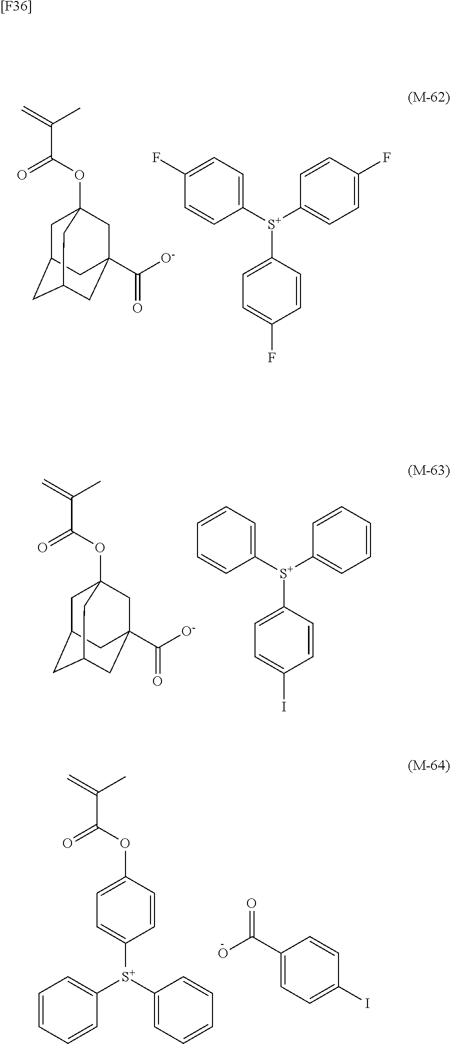

### Synthesis of Polymer (A)

### Synthesis Example 1

### Synthesis of Polymer (A-1)

The compound (M-1) and the compound (M-12) were dissolved in 2-butanone (200 parts by mass with respect to all the monomers) so as to adjust the mole ratio to 5/95. Azobisisobutyronitrile (AIBN) serving as a polymerization initiator was added thereto in an amount of 6 mol % with respect to all the monomers, to thereby prepare a monomer solution. Separately, 2-butanone (100 parts by mass) was added to a vacant reaction container and heated at 80° C. under stirring. Subsequently, the above-prepared monomer solution was added dropwise to the container over 3 hours, and the reaction was further heated at 80° C. for 3 hours. After completion of polymerization reaction, the polymerization solution was cooled to room temperature. To the thus-prepared polymerization solution, acetonitrile (100 parts by mass) and hexane (600 parts by mass) were added, and the mixture was stirred. The lower layer was separated, and the solvent was removed from the lower layer, to thereby yield a polymer (A-1). Table 1 shows Mw and Mw/Mn of the obtained polymer.

### Synthesis Examples 2 to 46

### Synthesis of Polymers (A-2) to (A-46)

The procedure of Synthesis Example 1 was repeated, except that the monomers of the type and amounts shown in Table 1 were used, to thereby yield polymers (A-2) to (A-46). Table 1 shows values of Mw and Mw/Mn of the obtained polymers.

TABLE 1

| | | First | | Second | | Additional | | | |
|---|---|---|---|---|---|---|---|---|---|
| | | structual | | structual | | structual | | | |
| | | unit | | unit | | unit | | Physical | |
| | Polymer | | Amount | | Amount | | Amount | properties | |
| | (A) | Type | (mol %) | Type | (mol %) | Type | (mol %) | Mw | Mw/Mn |
| Synthesis | A-1 | M-1 | 5 | M- | 95 | — | — | 9500 | 1.6 |
| Example | | | | 12 | | | | | |
| 1 | | | | | | | | | |
| Synthesis | A-2 | M-1 | 15 | M- | 85 | — | — | 9900 | 1.6 |
| Example | | | | 12 | | | | | |
| 2 | | | | | | | | | |
| Synthesis | A-3 | M-1 | 30 | M- | 70 | — | — | 9100 | 1.7 |
| Example | | | | 12 | | | | | |
| 3 | | | | | | | | | |
| Synthesis | A-4 | M-1 | 50 | M- | 50 | — | — | 8900 | 1.6 |
| Example | | | | 12 | | | | | |
| 4 | | | | | | | | | |
| Synthesis | A-5 | M-1 | 15 | M- | 85 | — | — | 4200 | 1.3 |
| Example | | | | 12 | | | | | |
| 5 | | | | | | | | | |
| Synthesis | A-6 | M-1 | 15 | M- | 85 | — | — | 15200 | 2.0 |
| Example | | | | 12 | | | | | |
| 6 | | | | | | | | | |
| Synthesis | A-7 | M-1 | 15 | M- | 85 | — | — | 19000 | 2.3 |
| Example | | | | 12 | | | | | |
| 7 | | | | | | | | | |
| Synthesis | A-8 | M-2 | 15 | M- | 85 | — | — | 8700 | 1.5 |
| Example | | | | 12 | | | | | |
| 8 | | | | | | | | | |
| Synthesis | A-9 | M-3 | 15 | M- | 85 | — | — | 9100 | 1.6 |
| Example | | | | 12 | | | | | |
| 9 | | | | | | | | | |
| Synthesis | A-10 | M-4 | 15 | M- | 85 | — | — | 9600 | 1.8 |
| Example | | | | 12 | | | | | |
| 10 | | | | | | | | | |
| Synthesis | A-11 | M-5 | 15 | M- | 85 | — | — | 7700 | 1.6 |
| Example | | | | 12 | | | | | |
| 11 | | | | | | | | | |
| Synthesis | A-12 | M-6 | 15 | M- | 85 | — | — | 9500 | 1.8 |
| Example | | | | 12 | | | | | |
| 12 | | | | | | | | | |
| Synthesis | A-13 | M-7 | 15 | M- | 85 | — | — | 11000 | 1.9 |
| Example | | | | 12 | | | | | |
| 13 | | | | | | | | | |
| Synthesis | A-14 | M-8 | 15 | M- | 85 | — | — | 8600 | 1.6 |
| Example | | | | 12 | | | | | |
| 14 | | | | | | | | | |
| Synthesis | A-15 | M-9 | 15 | M- | 85 | — | — | 9900 | 1.7 |
| Example | | | | 12 | | | | | |
| 15 | | | | | | | | | |
| Synthesis | A-16 | M-1 | 15 | M- | 85 | — | — | 7800 | 1.6 |
| Example | | | | 10 | | | | | |
| 16 | | | | | | | | | |
| Synthesis | A-17 | M-1 | 15 | M- | 85 | — | — | 8500 | 1.6 |
| Example | | | | 11 | | | | | |
| 17 | | | | | | | | | |
| Synthesis | A-18 | M-1 | 15 | M- | 85 | — | — | 9100 | 1.7 |
| Example | | | | 13 | | | | | |
| 18 | | | | | | | | | |
| Synthesis | A-19 | M-1 | 15 | M- | 85 | — | — | 9300 | 1.7 |
| Example | | | | 14 | | | | | |
| 19 | | | | | | | | | |
| Synthesis | A-20 | M-1 | 15 | M- | 85 | — | — | 7900 | 1.6 |
| Example | | | | 15 | | | | | |
| 20 | | | | | | | | | |
| Synthesis | A-21 | M-1 | 15 | M- | 85 | — | — | 8800 | 1.6 |
| Example | | | | 16 | | | | | |
| 21 | | | | | | | | | |
| Synthesis | A-22 | M-1 | 15 | M- | 85 | — | — | 7600 | 1.6 |
| Example | | | | 17 | | | | | |
| 22 | | | | | | | | | |
| Synthesis | A-23 | M-1 | 15 | M- | 85 | — | — | 9500 | 1.7 |
| Example | | | | 18 | | | | | |
| 23 | | | | | | | | | |
| Synthesis | A-24 | M-1 | 15 | M- | 85 | — | — | 8200 | 1.7 |
| Example | | | | 19 | | | | | |
| 24 | | | | | | | | | |
| Synthesis | A-25 | M-1 | 15 | M- | 85 | — | — | 7300 | 1.6 |
| Example | | | | 20 | | | | | |
| 25 | | | | | | | | | |
| Synthesis | A-26 | M-1 | 15 | M- | 85 | — | — | 9400 | 1.7 |
| Example | | | | 21 | | | | | |
| 26 | | | | | | | | | |
| Synthesis | A-27 | M-1 | 15 | M- | 85 | — | — | 8100 | 1.6 |
| Example | | | | 22 | | | | | |
| 27 | | | | | | | | | |
| Synthesis | A-28 | M-1 | 15 | M- | 70 | M- | 15 | 8300 | 1.6 |
| Example | | | | 12 | | 29 | | | |
| 28 | | | | | | | | | |
| Synthesis | A-29 | M-1 | 15 | M- | 70 | M- | 15 | 7500 | 1.5 |
| Example | | | | 12 | | 32 | | | |
| 29 | | | | | | | | | |
| Synthesis | A-30 | M-1 | 15 | M- | 70 | M- | 15 | 7800 | 1.5 |
| Example | | | | 12 | | 34 | | | |
| 30 | | | | | | | | | |
| Synthesis | A-31 | M-1 | 15 | M- | 70 | M- | 15 | 7700 | 1.5 |
| Example | | | | 12 | | 35 | | | |
| 31 | | | | | | | | | |
| Synthesis | A-32 | M-1 | 15 | M- | 70 | M- | 15 | 8500 | 1.9 |
| Example | | | | 12 | | 36 | | | |
| 32 | | | | | | | | | |
| Synthesis | A-33 | M-1 | 15 | M- | 70 | M- | 15 | 8800 | 1.6 |
| Example | | | | 12 | | 38 | | | |
| 33 | | | | | | | | | |
| Synthesis | A-34 | M-1 | 15 | M- | 70 | M- | 15 | 8700 | 1.7 |
| Example | | | | 12 | | 41 | | | |
| 34 | | | | | | | | | |
| Synthesis | A-35 | M-1 | 15 | M- | 70 | M- | 15 | 9100 | 1.8 |
| Example | | | | 12 | | 44 | | | |
| 35 | | | | | | | | | |
| Synthesis | A-36 | M-1 | 15 | M- | 70 | M- | 15 | 7500 | 1.5 |
| Example | | | | 12 | | 45 | | | |
| 36 | | | | | | | | | |
| Synthesis | A-37 | M-1 | 15 | M- | 70 | M- | 15 | 8300 | 1.6 |
| Example | | | | 12 | | 46 | | | |
| 37 | | | | | | | | | |
| Synthesis | A-38 | M-1 | 15 | M- | 70 | M- | 15 | 9100 | 1.8 |
| Example | | | | 12 | | 49 | | | |
| 38 | | | | | | | | | |
| Synthesis | A-39 | M-1 | 15 | M- | 70 | M- | 15 | 7700 | 1.7 |
| Example | | | | 12 | | 50 | | | |
| 39 | | | | | | | | | |
| Synthesis | A-40 | M-1 | 15 | M- | 70 | M- | 15 | 7600 | 1.6 |
| Example | | | | 12 | | 51 | | | |
| 40 | | | | | | | | | |
| Synthesis | A-41 | M-1 | 15 | M- | 70 | M- | 15 | 8200 | 1.7 |
| Example | | | | 12 | | 52 | | | |
| 41 | | | | | | | | | |
| Synthesis | A-42 | M-1 | 15 | M- | 70 | M- | 15 | 8800 | 1.8 |
| Example | | | | 12 | | 53 | | | |
| 42 | | | | | | | | | |
| Synthesis | A-43 | M-1 | 15 | M- | 70 | M- | 15 | 8400 | 1.7 |
| Example | | | | 12 | | 54 | | | |
| 43 | | | | | | | | | |
| Synthesis | A-44 | — | — | M- | 100 | — | — | 7500 | 1.5 |
| Example | | | | 12 | | | | | |
| 44 | | | | | | | | | |
| Synthesis | A-45 | — | — | M- | 70 | M- | 30 | 7100 | 1.5 |
| Example | | | | 12 | | 29 | | | |
| 45 | | | | | | | | | |
| Synthesis | A-46 | — | — | M- | 70 | M- | 30 | 7600 | 1.6 |
| Example | | | | 12 | | 51 | | | |
| 46 | | | | | | | | | |

### Synthesis of Polymer(C)

### Synthesis Example 47

### Synthesis of Polymer (C-1)

The compound (M-35) and the compound (M-23) were dissolved in methanol (200 parts by mass with respect to all the monomers) so as to adjust the mole ratio to 70/30. AIBN serving as a polymerization initiator was added thereto in an amount of 6 mol % with respect to all the monomers, to thereby prepare a monomer solution. Separately, 1-methoxy-2-propanol (100 parts by mass with respect to all the monomers) was added to a vacant reaction container and heated at 85° C. under stirring. Subsequently, the above-prepared monomer solution was added dropwise to the container over 3 hours, and the reaction was further heated at 85° C. for 3 hours. After completion of polymerization reaction, the polymerization solution was cooled to room temperature.

The thus-cooled polymerization solution was put into hexane (500 parts by mass with respect to the polymerization solution), and precipitated white powder was separated through filtration. The white powder separated through filtration was washed twice with hexane (100 parts by mass with respect to the polymerization solution), and the washed powder was dissolved again in 1-methoxy-2-propanol (300 parts by mass). Subsequently, while methanol (500 parts by mass), triethylamine (50 parts by mass), and ultra-pure water (10 parts by mass) were added to the solution, the mixture was subjected to hydrolysis at 70° C. for 6 hours under stirring.

After completion of reaction, the residual solvent was removed through distillation, and the resultant solid was dissolved in acetone (100 parts by mass). The solution was added dropwise to water (500 parts by mass), to thereby solidify the resin, and the formed solid was separated through filtration. The solid was dried at 50° C. for 12 hours, to thereby yield a polymer (C-1) as a white powder. Table 2 shows Mw and Mw/Mn of the obtained polymer.

### Synthesis Examples 48 to 86

### Synthesis of Polymers (C-2) to (C-40)

The procedure of Synthesis Example 47 was repeated, except that the monomers of the type and amounts shown in Table 2 were used, to thereby yield polymers (C-2) to (C-40). Table 2 shows values of Mw and Mw/Mn of the obtained polymers.

TABLE 2

| | | Third | | Fourth | | Additional | | | |
|---|---|---|---|---|---|---|---|---|---|
| | | structual | | structual | | structual | | | |
| | | unit(s) | | unit(s) | | unit | | Physical | |
| | Polymer | | Amount | | Amount | | Amount | properties | |
| | (C) | Type | (mol %) | Type | (mol %) | Type | (mol %) | Mw | Mw/Mn |
| Synthesis | C-1 | M-35 | 70 | M-23 | 30 | — | — | 5800 | 1.4 |
| Example | | | | | | | | | |
| 47 | | | | | | | | | |
| Synthesis | C-2 | M-35 | 50 | M-23 | 50 | | | 6200 | 1.5 |
| Example | | | | | | | | | |
| 48 | | | | | | | | | |
| Synthesis | C-3 | M-35 | 30 | M-23 | 70 | — | — | 5900 | 1.4 |
| Example | | | | | | | | | |
| 49 | | | | | | | | | |
| Synthesis | C-4 | M-35 | 50 | M-23 | 50 | — | — | 3100 | 1.3 |
| Example | | | | | | | | | |
| 50 | | | | | | | | | |
| Synthesis | C-5 | M-35 | 50 | M-23 | 50 | — | — | 8900 | 1.6 |
| Example | | | | | | | | | |
| 51 | | | | | | | | | |
| Synthesis | C-6 | M-35 | 50 | M-23 | 50 | — | — | 11000 | 1.8 |
| Example | | | | | | | | | |
| 52 | | | | | | | | | |
| Synthesis | C-7 | M-35 | 50 | M-23/M- | 25/25 | — | — | 8700 | 1.5 |
| Example | | | | 24 | | | | | |
| 53 | | | | | | | | | |
| Synthesis | C-8 | M-35 | 50 | M-23/M- | 25/25 | — | — | 6200 | 1.4 |
| Example | | | | 25 | | | | | |
| 54 | | | | | | | | | |
| Synthesis | C-9 | M-35 | 50 | M-23/M- | 25/25 | — | — | 6600 | 1.5 |
| Example | | | | 26 | | | | | |
| 55 | | | | | | | | | |
| Synthesis | C-10 | M-35 | 50 | M-23/M- | 25/25 | — | — | 5400 | 1.4 |
| Example | | | | 27 | | | | | |
| 56 | | | | | | | | | |
| Synthesis | C-11 | M-35 | 50 | M-23/M- | 25/25 | — | — | 5500 | 1.3 |
| Example | | | | 28 | | | | | |
| 57 | | | | | | | | | |
| Synthesis | C-12 | M-35 | 50 | M-23/M- | 25/25 | — | — | 5700 | 1.4 |
| Example | | | | 29 | | | | | |
| 58 | | | | | | | | | |
| Synthesis | C-13 | M-35 | 50 | M-23/M- | 25/25 | — | — | 6100 | 1.4 |
| Example | | | | 30 | | | | | |
| 59 | | | | | | | | | |
| Synthesis | C-14 | M-35 | 50 | M-23/M- | 25/25 | — | — | 6300 | 1.5 |
| Example | | | | 31 | | | | | |
| 60 | | | | | | | | | |
| Synthesis | C-15 | M-35 | 50 | M-23/M- | 25/25 | — | — | 5800 | 1.4 |
| Example | | | | 32 | | | | | |
| 61 | | | | | | | | | |
| Synthesis | C-16 | M-35 | 50 | M-23/M- | 25/25 | — | — | 5100 | 1.4 |
| Example | | | | 33 | | | | | |
| 62 | | | | | | | | | |
| Synthesis | C-17 | M-35 | 50 | M-23/M- | 25/25 | — | — | 5500 | 1.4 |
| Example | | | | 34 | | | | | |
| 63 | | | | | | | | | |
| Synthesis | C-18 | M-36 | 50 | M-23 | 50 | — | — | 5400 | 1.6 |
| Example | | | | | | | | | |
| 64 | | | | | | | | | |
| Synthesis | C-19 | M-37 | 50 | M-23 | 50 | — | — | 6000 | 1.4 |
| Example | | | | | | | | | |
| 65 | | | | | | | | | |
| Synthesis | C-20 | M-38 | 50 | M-23 | 50 | — | — | 6200 | 1.5 |
| Example | | | | | | | | | |
| 66 | | | | | | | | | |
| Synthesis | C-21 | M-39 | 50 | M-23 | 50 | — | — | 6200 | 1.5 |
| Example | | | | | | | | | |
| 67 | | | | | | | | | |
| Synthesis | C-22 | M-40 | 50 | M-23 | 50 | — | — | 5800 | 1.4 |
| Example | | | | | | | | | |
| 68 | | | | | | | | | |
| Synthesis | C-23 | M-41 | 50 | M-23 | 50 | — | — | 5900 | 1.4 |
| Example | | | | | | | | | |
| 69 | | | | | | | | | |
| Synthesis | C-24 | M-42 | 50 | M-23 | 50 | — | — | 5500 | 1.4 |
| Example | | | | | | | | | |
| 70 | | | | | | | | | |
| Synthesis | C-25 | M-43 | 50 | M-23 | 50 | — | — | 5600 | 1.4 |
| Example | | | | | | | | | |
| 71 | | | | | | | | | |
| Synthesis | C-26 | M-44 | 50 | M-23 | 50 | — | — | 5200 | 1.3 |
| Example | | | | | | | | | |
| 72 | | | | | | | | | |
| Synthesis | C-27 | M-45 | 50 | M-23 | 50 | — | — | 6100 | 1.5 |
| Example | | | | | | | | | |
| 73 | | | | | | | | | |
| Synthesis | C-28 | M-46 | 50 | M-23 | 50 | — | — | 6600 | 1.5 |
| Example | | | | | | | | | |
| 74 | | | | | | | | | |
| Synthesis | C-29 | M-47 | 50 | M-23 | 50 | — | — | 6500 | 1.5 |
| Example | | | | | | | | | |
| 75 | | | | | | | | | |
| Synthesis | C-30 | M-35 | 25 | M-23 | 50 | M- | 25 | 6300 | 1.5 |
| Example | | | | | | 48 | | | |
| 76 | | | | | | | | | |
| Synthesis | C-31 | M-35 | 25 | M-23 | 50 | M- | 25 | 5800 | 1.4 |
| Example | | | | | | 49 | | | |
| 77 | | | | | | | | | |
| Synthesis | C-32 | M-35 | 50 | M-23 | 25 | M- | 25 | 5400 | 1.4 |
| Example | | | | | | 50 | | | |
| 78 | | | | | | | | | |
| Synthesis | C-33 | M-35 | 50 | M-23 | 25 | M- | 25 | 5500 | 1.3 |
| Example | | | | | | 51 | | | |
| 79 | | | | | | | | | |
| Synthesis | C-34 | M-35 | 50 | M-23 | 25 | M- | 25 | 5700 | 1.4 |
| Example | | | | | | 52 | | | |
| 80 | | | | | | | | | |
| Synthesis | C-35 | M-35 | 50 | M-23 | 25 | M- | 25 | 6200 | 1.5 |
| Example | | | | | | 53 | | | |
| 81 | | | | | | | | | |
| Synthesis | C-36 | M-35 | 50 | M-23 | 25 | M- | 25 | 5400 | 1.6 |
| Example | | | | | | 54 | | | |
| 82 | | | | | | | | | |
| Synthesis | C-37 | M-35/M- | 25/25 | M-23 | 50 | — | — | 5800 | 1.4 |
| Example | | 55 | | | | | | | |
| 83 | | | | | | | | | |
| Synthesis | C-38 | M-55 | 50 | M-23 | 50 | — | — | 4800 | 1.3 |
| Example | | | | | | | | | |
| 84 | | | | | | | | | |
| Synthesis | C-39 | M-35/M- | 25/25 | M-23 | 50 | — | — | 5700 | 1.4 |
| Example | | 56 | | | | | | | |
| 85 | | | | | | | | | |
| Synthesis | C-40 | M-56 | 50 | M-23 | 50 | — | — | 5900 | 1.4 |
| Example | | | | | | | | | |
| 86 | | | | | | | | | |

### Synthesis Example 87

### Synthesis of Polymer (C-41)

The compound (M-29), the compound (M-35), and the compound (M-58) were dissolved in 2-butanone (200 parts by mass with respect to all the monomers) so as to adjust the mole proportions to 30/50/20. Azobisisobutyronitrile (AIBN) serving as a polymerization initiator was added thereto in an amount of 6 mol % with respect to all the monomers, to thereby prepare a monomer solution. Separately, 2-butanone (100 parts by mass with respect to all the monomers) was added to a vacant reaction container and heated at 80° C. under stirring. Subsequently, the above-prepared monomer solution was added dropwise to the container over 3 hours, and the reaction was further heated at 80° C. for 3 hours. After completion of polymerization reaction, the polymerization solution was cooled to room temperature.

To the thus-cooled polymerization solution, acetonitrile (100 parts by mass) and hexane (600 parts by mass) were added, and the mixture was stirred. The lower layer was separated, and the solvent was removed from the lower layer, to thereby yield a polymer (C-41).

### Synthesis Examples 88 to 100

### Synthesis of Polymers (C-42) to (C-54)

The procedure of Synthesis Example 87 was repeated, except that the monomers of the type and amounts shown in Table 3 were used, to thereby yield polymers (C-42) to (C-54). Table 3 shows values of Mw and Mw/Mn of the obtained polymers.

TABLE 3

| | | Third | | Fourth | | Additional | | | |
|---|---|---|---|---|---|---|---|---|---|
| | | structual | | structual | | structual | | | |
| | | unit(s) | | unit | | unit(s) | | Physical | |
| | Polymer | | Amount | | Amount | | Amount | properties | |
| | (C) | Type | (mol %) | Type | (mol %) | Type | (mol %) | Mw | Mw/Mn |
| Synthesis | C-41 | M-35 | 50 | M- | 30 | M-58 | 20 | 6100 | 1.4 |
| Example | | | | 29 | | | | | |
| 87 | | | | | | | | | |
| Synthesis | C-42 | M-45 | 50 | M- | 30 | M-58 | 20 | 6600 | 1.4 |
| Example | | | | 29 | | | | | |
| 88 | | | | | | | | | |
| Synthesis | C-43 | M-55 | 50 | M- | 30 | M-58 | 20 | 6400 | 1.4 |
| Example | | | | 29 | | | | | |
| 89 | | | | | | | | | |
| Synthesis | C-44 | M-57 | 50 | M- | 30 | M-58 | 20 | 5900 | 1.3 |
| Example | | | | 29 | | | | | |
| 90 | | | | | | | | | |
| Synthesis | C-45 | M-35/M- | 25/25 | M- | 30 | M-58 | 20 | 6800 | 1.3 |
| Example | | 45 | | 29 | | | | | |
| 91 | | | | | | | | | |
| Synthesis | C-46 | M-35/M- | 25/25 | M- | 30 | M-58 | 20 | 6500 | 1.3 |
| Example | | 55 | | 29 | | | | | |
| 92 | | | | | | | | | |
| Synthesis | C-47 | M-35/M- | 25/25 | M- | 30 | M-58 | 20 | 6200 | 1.4 |
| Example | | 57 | | 29 | | | | | |
| 93 | | | | | | | | | |
| Synthesis | C-48 | M-35 | 50 | M- | 30 | M-59 | 20 | 6200 | 1.4 |
| Example | | | | 29 | | | | | |
| 94 | | | | | | | | | |
| Synthesis | C-49 | M-35 | 50 | M- | 30 | M-60 | 20 | 5300 | 1.3 |
| Example | | | | 29 | | | | | |
| 95 | | | | | | | | | |
| Synthesis | C-50 | M-35 | 50 | M- | 30 | M-61 | 20 | 5500 | 1.3 |
| Example | | | | 29 | | | | | |
| 96 | | | | | | | | | |
| Synthesis | C-51 | M-35 | 50 | M- | 30 | M-62 | 20 | 5900 | 1.4 |
| Example | | | | 29 | | | | | |
| 97 | | | | | | | | | |
| Synthesis | C-52 | M-35 | 50 | M- | 30 | M-63 | 20 | 6100 | 1.4 |
| Example | | | | 29 | | | | | |
| 98 | | | | | | | | | |
| Synthesis | C-53 | M-35 | 50 | M- | 30 | M-64 | 20 | 6200 | 1.4 |
| Example | | | | 29 | | | | | |
| 99 | | | | | | | | | |
| Synthesis | C-54 | M-35 | 50 | M- | 30 | M-58/M- | 10/10 | 6000 | 1.4 |
| Example | | | | 29 | | 62 | | | |
| 100 | | | | | | | | | |

### <Preparation of Radiation-Sensitive Composition>

The acid-generating agent (PAG), acid diffusion control agent, and solvent used in preparation of radiation-sensitive compositions are as follows.

### [Acid-Generating Agent]

### Bp-1 to Bp-16: Compounds Represented by the Following Formulas (Bp-1) to (Bp-16).

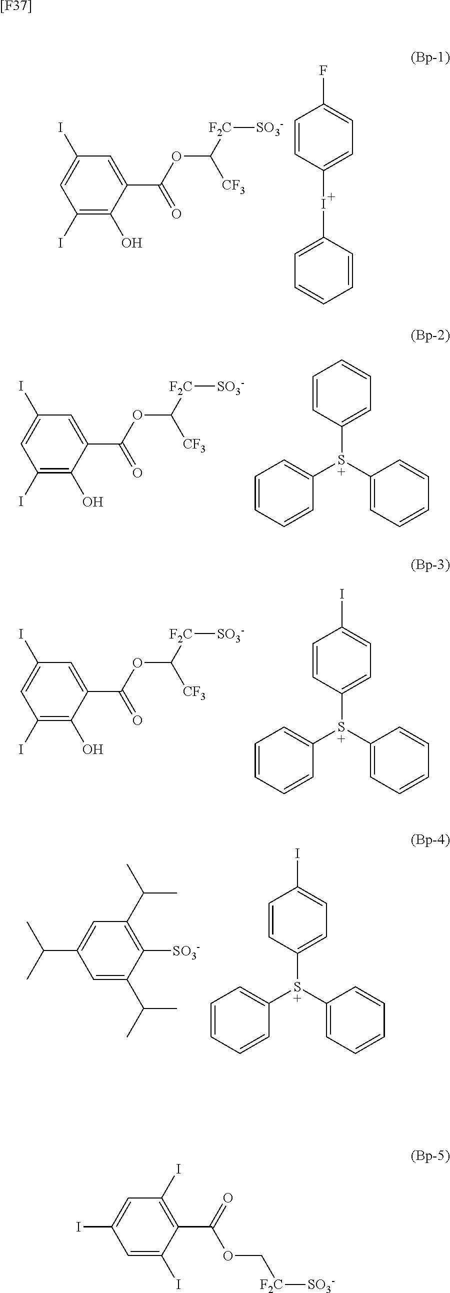

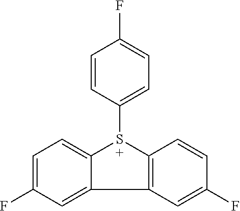

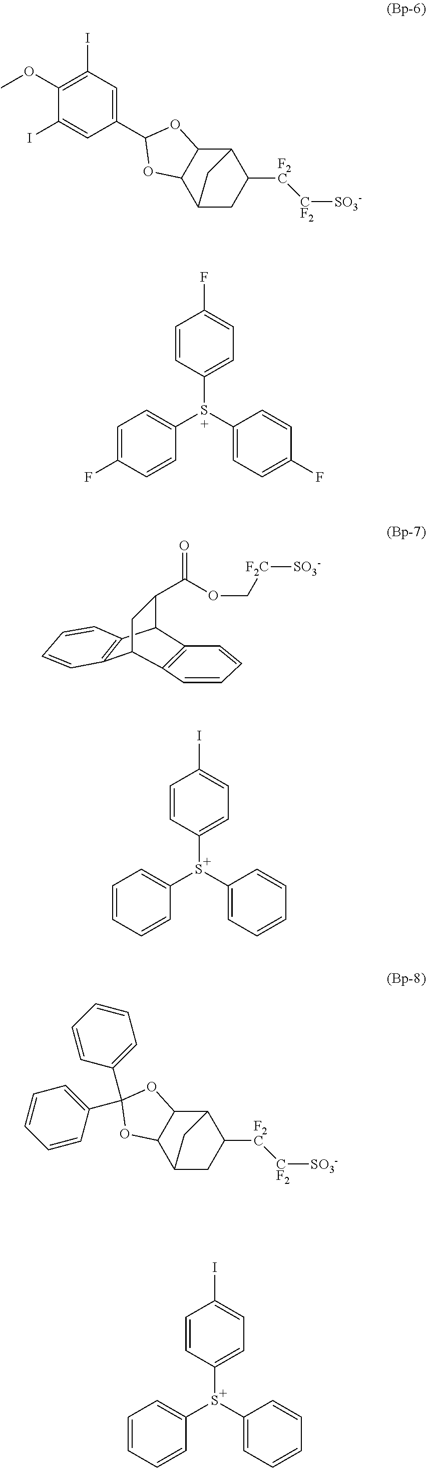

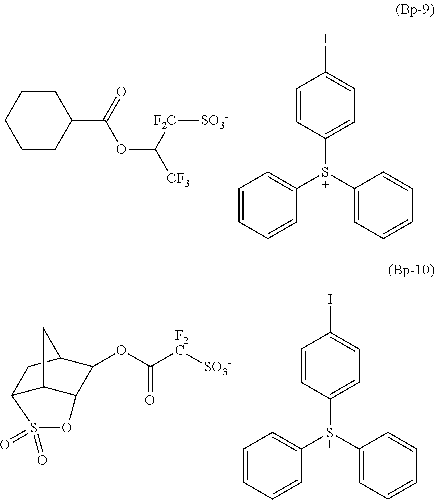

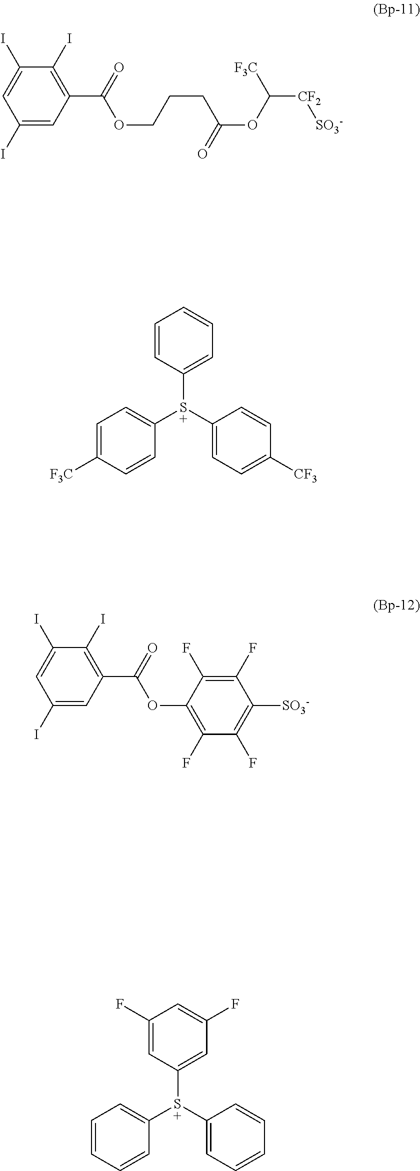

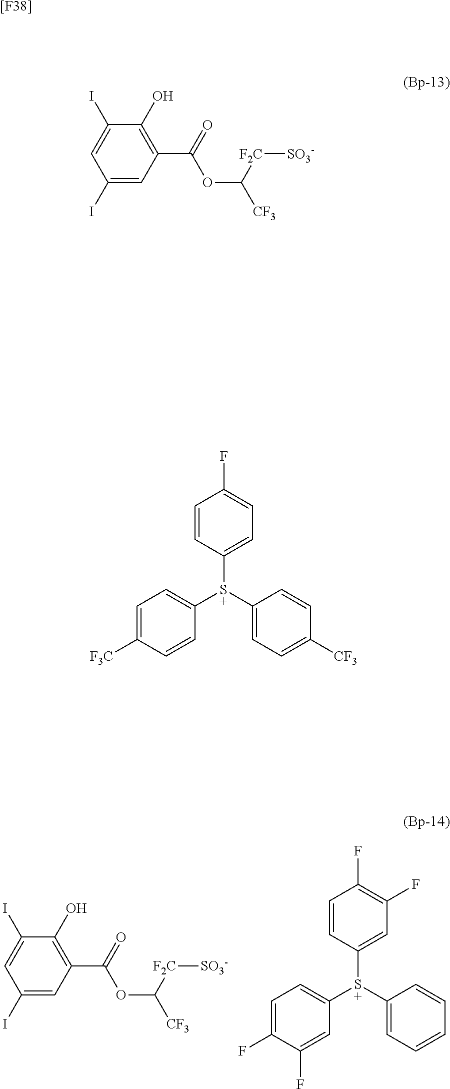

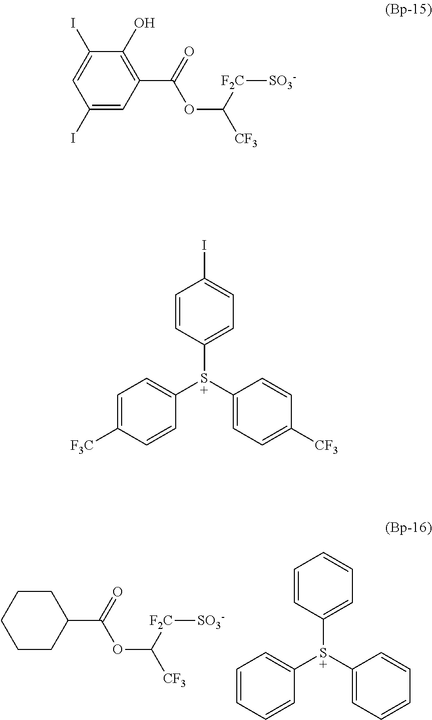

### [Acid Diffusion Control Agent]

### Bq-1 to Bq-16: Compounds Represented by the Following Formulas (Bq-1) to (Bq-16).

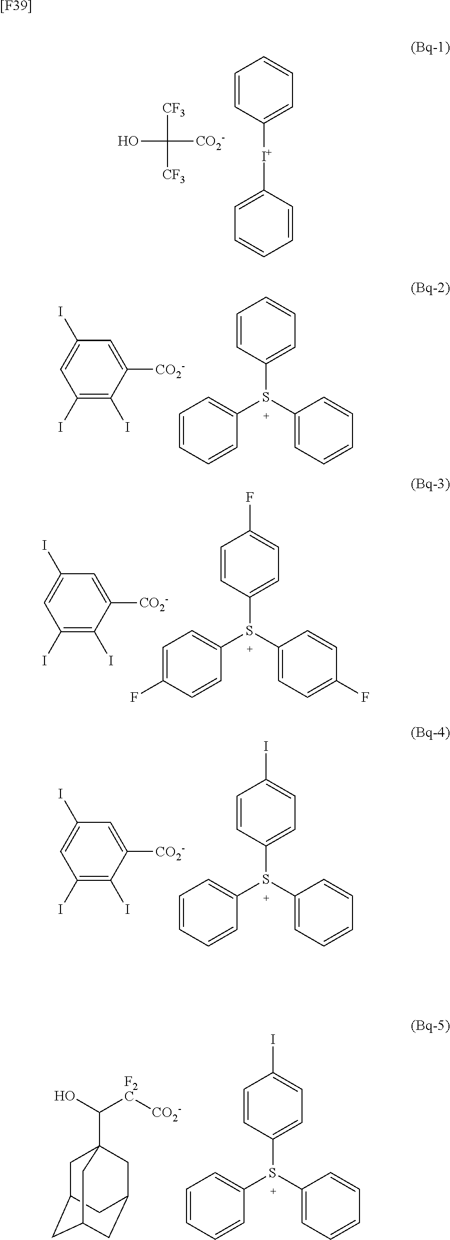

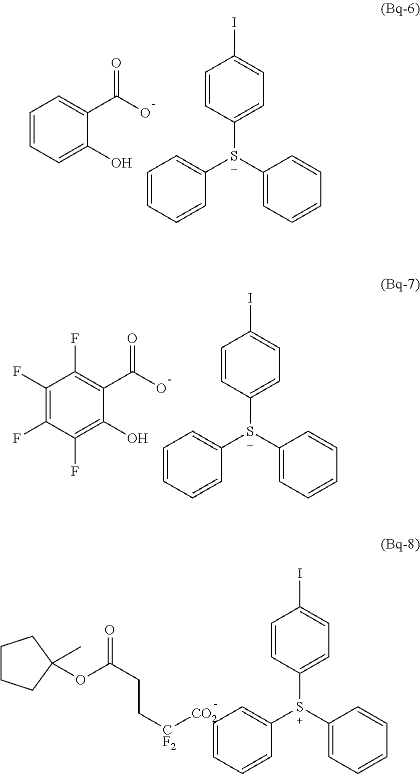

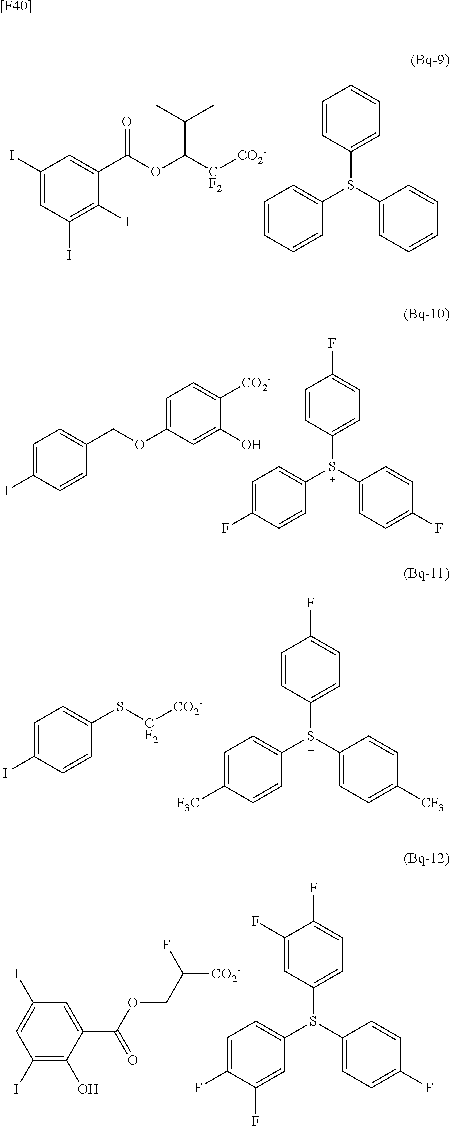

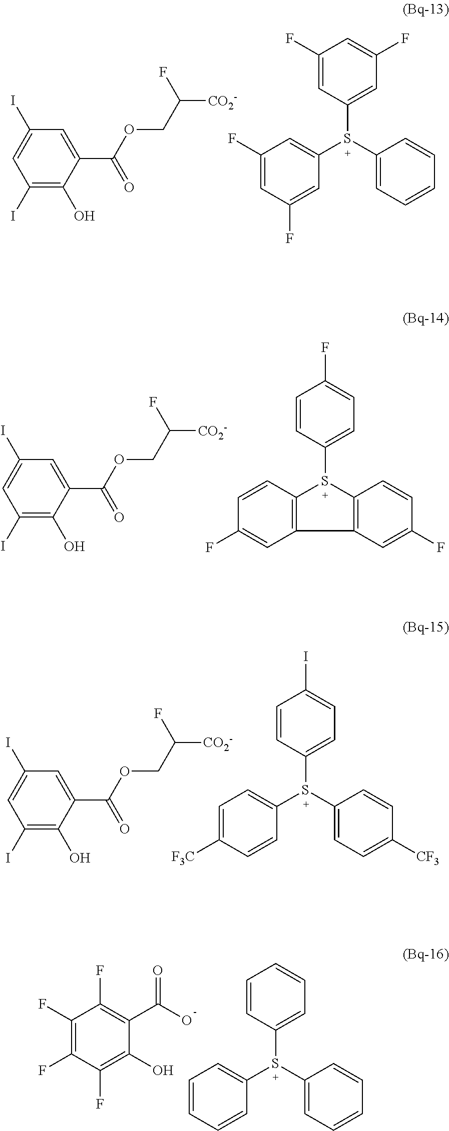

### [Solvent]

E-1: Propylene glycol monomethyl ether acetate
E-2: Propylene glycol 1-monomethyl ether

### Example 1

The polymer (A-2) (1 part by mass), the polymer (C-2) (100 parts by mass), the acid-generating agent (Bp-1) (20 parts by mass), the acid diffusion control agent (Bq-1) (20 mol % with respect to the acid-generating agent (Bp-1)), the solvent (E-1) (4,800 parts by mass), and the solvent (E-2) (2,000 parts by mass) were blended with mixing. Then, the liquid mixture was filtered through a membrane filter (pore size: 0.20 m), to thereby prepare a radiation-sensitive composition (R-1).

### Examples 2 to 128 and Comparative Examples 1 to 4

The procedure of Example 1 was repeated, except that the components of the type and amounts shown in the following tables 4, 5, 6, and 7 were used, to thereby prepare radiation-sensitive compositions (R-2) to (R-128) and (CR-1) to (CR-4), respectively. In Tables 4 to 7, the amount of the acid diffusion control agent corresponds to a ratio (mol %) with respect to the amount of the acid-generating agent.

TABLE 4

| | | | | | | | | Acid diffusion | | | |
|---|---|---|---|---|---|---|---|---|---|---|---|
| | | | | | | | | control agent | | | |
| | | | | | | | | | ratio | | |
| | | | | | | | | | (mol %) | | |
| | | | | | | | | | with | | |
| | | | | | | Acid-generating | | | respect to | | |
| | | Polymer (A) | | Polymer (C) | | agent | | | amount | Solvent | |
| | Radiation- | | Part(s) | | Part(s) | | Part(s) | | of acig- | | Part(s) |
| | sensitive | | by | | by | | by | | generating | | by |
| | composition | Type | mass | Type | mass | Type | mass | Type | agent | Type | mass |
| Example | R-1 | A-2 | 1 | C-2 | 100 | Bp-1 | 20 | Bq-1 | 20 | E-1/E-2 | 4800/2000 |
| 1 | | | | | | | | | | | |
| Example | R-2 | A-2 | 7 | C-2 | 100 | Bp-1 | 20 | Bq-1 | 20 | E-1/E-2 | 4800/2000 |
| 2 | | | | | | | | | | | |
| Example | R-3 | A-2 | 15 | C-2 | 100 | Bp-1 | 20 | Bq-1 | 20 | E-1/E-2 | 4800/2000 |
| 3 | | | | | | | | | | | |
| Example | R-4 | A-1 | 7 | C-2 | 100 | Bp-1 | 20 | Bq-1 | 20 | E-1/E-2 | 4800/2000 |
| 4 | | | | | | | | | | | |
| Example | R-5 | A-3 | 7 | C-2 | 100 | Bp-1 | 20 | Bq-1 | 20 | E-1/E-2 | 4800/2000 |
| 5 | | | | | | | | | | | |
| Example | R-6 | A-4 | 7 | C-2 | 100 | Bp-1 | 20 | Bq-1 | 20 | E-1/E-2 | 4800/2000 |
| 6 | | | | | | | | | | | |
| Example | R-7 | A-5 | 7 | C-2 | 100 | Bp-1 | 20 | Bq-1 | 20 | E-1/E-2 | 4800/2000 |
| 7 | | | | | | | | | | | |
| Example | R-8 | A-6 | 7 | C-2 | 100 | Bp-1 | 20 | Bq-1 | 20 | E-1/E-2 | 4800/2000 |
| 8 | | | | | | | | | | | |
| Example | R-9 | A-7 | 7 | C-2 | 100 | Bp-1 | 20 | Bq-1 | 20 | E-1/E-2 | 4800/2000 |
| 9 | | | | | | | | | | | |
| Example | R-10 | A-8 | 7 | C-2 | 100 | Bp-1 | 20 | Bq-1 | 20 | E-1/E-2 | 4800/2000 |
| 10 | | | | | | | | | | | |
| Example | R-11 | A-9 | 7 | C-2 | 100 | Bp-1 | 20 | Bq-1 | 20 | E-1/E-2 | 4800/2000 |
| 11 | | | | | | | | | | | |
| Example | R-12 | A-10 | 7 | C-2 | 100 | Bp-1 | 20 | Bq-1 | 20 | E-1/E-2 | 4800/2000 |
| 12 | | | | | | | | | | | |
| Example | R-13 | A-11 | 7 | C-2 | 100 | Bp-1 | 20 | Bq-1 | 20 | E-1/E-2 | 4800/2000 |
| 13 | | | | | | | | | | | |
| Example | R-14 | A-12 | 7 | C-2 | 100 | Bp-1 | 20 | Bq-1 | 20 | E-1/E-2 | 4800/2000 |
| 14 | | | | | | | | | | | |
| Example | R-15 | A-13 | 7 | C-2 | 100 | Bp-1 | 20 | Bq-1 | 20 | E-1/E-2 | 4800/2000 |
| 15 | | | | | | | | | | | |
| Example | R-16 | A-14 | 7 | C-2 | 100 | Bp-1 | 20 | Bq-1 | 20 | E-1/E-2 | 4800/2000 |
| 16 | | | | | | | | | | | |
| Example | R-17 | A-15 | 7 | C-2 | 100 | Bp-1 | 20 | Bq-1 | 20 | E-1/E-2 | 4800/2000 |
| 17 | | | | | | | | | | | |
| Example | R-18 | A-16 | 7 | C-2 | 100 | Bp-1 | 20 | Bq-1 | 20 | E-1/E-2 | 4800/2000 |
| 18 | | | | | | | | | | | |
| Example | R-19 | A-17 | 7 | C-2 | 100 | Bp-1 | 20 | Bq-1 | 20 | E-1/E-2 | 4800/2000 |
| 19 | | | | | | | | | | | |
| Example | R-20 | A-18 | 7 | C-2 | 100 | Bp-1 | 20 | Bq-1 | 20 | E-1/E-2 | 4800/2000 |
| 20 | | | | | | | | | | | |
| Example | R-21 | A-19 | 7 | C-2 | 100 | Bp-1 | 20 | Bq-1 | 20 | E-1/E-2 | 4800/2000 |
| 21 | | | | | | | | | | | |
| Example | R-22 | A-20 | 7 | C-2 | 100 | Bp-1 | 20 | Bq-1 | 20 | E-1/E-2 | 4800/2000 |
| 22 | | | | | | | | | | | |
| Example | R-23 | A-21 | 7 | C-2 | 100 | Bp-1 | 20 | Bq-1 | 20 | E-1/E-2 | 4800/2000 |
| 23 | | | | | | | | | | | |
| Example | R-24 | A-22 | 7 | C-2 | 100 | Bp-1 | 20 | Bq-1 | 20 | E-1/E-2 | 4800/2000 |
| 24 | | | | | | | | | | | |
| Example | R-25 | A-23 | 7 | C-2 | 100 | Bp-1 | 20 | Bq-1 | 20 | E-1/E-2 | 4800/2000 |
| 25 | | | | | | | | | | | |
| Example | R-26 | A-24 | 7 | C-2 | 100 | Bp-1 | 20 | Bq-1 | 20 | E-1/E-2 | 4800/2000 |
| 26 | | | | | | | | | | | |
| Example | R-27 | A-25 | 7 | C-2 | 100 | Bp-1 | 20 | Bq-1 | 20 | E-1/E-2 | 4800/2000 |
| 27 | | | | | | | | | | | |
| Example | R-28 | A-26 | 7 | C-2 | 100 | Bp-1 | 20 | Bq-1 | 20 | E-1/E-2 | 4800/2000 |
| 28 | | | | | | | | | | | |
| Example | R-29 | A-27 | 7 | C-2 | 100 | Bp-1 | 20 | Bq-1 | 20 | E-1/E-2 | 4800/2000 |
| 29 | | | | | | | | | | | |
| Example | R-30 | A-28 | 7 | C-2 | 100 | Bp-1 | 20 | Bq-1 | 20 | E-1/E-2 | 4800/2000 |
| 30 | | | | | | | | | | | |
| Example | R-31 | A-29 | 7 | C-2 | 100 | Bp-1 | 20 | Bq-1 | 20 | E-1/E-2 | 4800/2000 |
| 31 | | | | | | | | | | | |
| Example | R-32 | A-30 | 7 | C-2 | 100 | Bp-1 | 20 | Bq-1 | 20 | E-1/E-2 | 4800/2000 |
| 32 | | | | | | | | | | | |
| Example | R-33 | A-31 | 7 | C-2 | 100 | Bp-1 | 20 | Bq-1 | 20 | E-1/E-2 | 4800/2000 |
| 33 | | | | | | | | | | | |
| Example | R-34 | A-32 | 7 | C-2 | 100 | Bp-1 | 20 | Bq-1 | 20 | E-1/E-2 | 4800/2000 |
| 34 | | | | | | | | | | | |
| Example | R-35 | A-33 | 7 | C-2 | 100 | Bp-1 | 20 | Bq-1 | 20 | E-1/E-2 | 4800/2000 |
| 35 | | | | | | | | | | | |

TABLE 5

| | | | | | | | | Acid diffusion | | | |
|---|---|---|---|---|---|---|---|---|---|---|---|
| | | | | | | | | control agent | | | |
| | | | | | | | | | ratio | | |
| | | | | | | | | | (mol %) | | |
| | | | | | | | | | with | | |
| | | | | | | Acid-generating | | | respect to | | |
| | | Polymer (A) | | Polymer (C) | | agent | | | amount of | | |
| | | | Part(s) | | Part(s) | | Part(s) | | acig- | Solvent | |
| | Radiation- | | by | | by | | by | | generating | | Part(s) by |
| | composition | Type | mass | Type | mass | Type | mass | Type | agent | Type | mass |
| Example | R-36 | A-34 | 7 | C-2 | 100 | Bp-1 | 20 | Bq-1 | 20 | E-1/E-2 | 4800/2000 |
| 36 | | | | | | | | | | | |
| Example | R-37 | A-35 | 7 | C-2 | 100 | Bp-1 | 20 | Bq-1 | 20 | E-1/E-2 | 4800/2000 |
| 37 | | | | | | | | | | | |
| Example | R-38 | A-36 | 7 | C-2 | 100 | Bp-1 | 20 | Bq-1 | 20 | E-1/E-2 | 4800/2000 |
| 38 | | | | | | | | | | | |
| Example | R-39 | A-37 | 7 | C-2 | 100 | Bp-1 | 20 | Bq-1 | 20 | E-1/E-2 | 4800/2000 |
| 39 | | | | | | | | | | | |
| Example | R-40 | A-38 | 7 | C-2 | 100 | Bp-1 | 20 | Bq-1 | 20 | E-1/E-2 | 4800/2000 |
| 40 | | | | | | | | | | | |
| Example | R-41 | A-39 | 7 | C-2 | 100 | Bp-1 | 20 | Bq-1 | 20 | E-1/E-2 | 4800/2000 |
| 41 | | | | | | | | | | | |
| Example | R-42 | A-40 | 7 | C-2 | 100 | Bp-1 | 20 | Bq-1 | 20 | E-1/E-2 | 4800/2000 |
| 42 | | | | | | | | | | | |
| Example | R-43 | A-41 | 7 | C-2 | 100 | Bp-1 | 20 | Bq-1 | 20 | E-1/E-2 | 4800/2000 |
| 43 | | | | | | | | | | | |
| Example | R-44 | A-42 | 7 | C-2 | 100 | Bp-1 | 20 | Bq-1 | 20 | E-1/E-2 | 4800/2000 |
| 44 | | | | | | | | | | | |
| Example | R-45 | A-43 | 7 | C-2 | 100 | Bp-1 | 20 | Bq-1 | 20 | E-1/E-2 | 4800/2000 |
| 45 | | | | | | | | | | | |
| Example | R-46 | A-2 | 7 | C-1 | 100 | Bp-1 | 20 | Bq-1 | 20 | E-1/E-2 | 4800/2000 |
| 46 | | | | | | | | | | | |
| Example | R-47 | A-2 | 7 | C-3 | 100 | Bp-1 | 20 | Bq-1 | 20 | E-1/E-2 | 4800/2000 |
| 47 | | | | | | | | | | | |
| Example | R-48 | A-2 | 7 | C-4 | 100 | Bp-1 | 20 | Bq-1 | 20 | E-1/E-2 | 4800/2000 |
| 48 | | | | | | | | | | | |
| Example | R-49 | A-2 | 7 | C-5 | 100 | Bp-1 | 20 | Bq-1 | 20 | E-1/E-2 | 4800/2000 |
| 49 | | | | | | | | | | | |
| Example | R-50 | A-2 | 7 | C-6 | 100 | Bp-1 | 20 | Bq-1 | 20 | E-1/E-2 | 4800/2000 |
| 50 | | | | | | | | | | | |
| Example | R-51 | A-2 | 7 | C-7 | 100 | Bp-1 | 20 | Bq-1 | 20 | E-1/E-2 | 4800/2000 |
| 51 | | | | | | | | | | | |
| Example | R-52 | A-2 | 7 | C-8 | 100 | Bp-1 | 20 | Bq-1 | 20 | E-1/E-2 | 4800/2000 |
| 52 | | | | | | | | | | | |
| Example | R-53 | A-2 | 7 | C-9 | 100 | Bp-1 | 20 | Bq-1 | 20 | E-1/E-2 | 4800/2000 |
| 53 | | | | | | | | | | | |
| Example | R-54 | A-2 | 7 | C-10 | 100 | Bp-1 | 20 | Bq-1 | 20 | E-1/E-2 | 4800/2000 |
| 54 | | | | | | | | | | | |
| Example | R-55 | A-2 | 7 | C-11 | 100 | Bp-1 | 20 | Bq-1 | 20 | E-1/E-2 | 4800/2000 |
| 55 | | | | | | | | | | | |
| Example | R-56 | A-2 | 7 | C-12 | 100 | Bp-1 | 20 | Bq-1 | 20 | E-1/E-2 | 4800/2000 |
| 56 | | | | | | | | | | | |
| Example | R-57 | A-2 | 7 | C-13 | 100 | Bp-1 | 20 | Bq-1 | 20 | E-1/E-2 | 4800/2000 |
| 57 | | | | | | | | | | | |
| Example | R-58 | A-2 | 7 | C-14 | 100 | Bp-1 | 20 | Bq-1 | 20 | E-1/E-2 | 4800/2000 |
| 58 | | | | | | | | | | | |
| Example | R-59 | A-2 | 7 | C-15 | 100 | Bp-1 | 20 | Bq-1 | 20 | E-1/E-2 | 4800/2000 |
| 59 | | | | | | | | | | | |
| Example | R-60 | A-2 | 7 | C-16 | 100 | Bp-1 | 20 | Bq-1 | 20 | E-1/E-2 | 4800/2000 |
| 60 | | | | | | | | | | | |
| Example | R-61 | A-2 | 7 | C-17 | 100 | Bp-1 | 20 | Bq-1 | 20 | E-1/E-2 | 4800/2000 |
| 61 | | | | | | | | | | | |
| Example | R-62 | A-2 | 7 | C-18 | 100 | Bp-1 | 20 | Bq-1 | 20 | E-1/E-2 | 4800/2000 |
| 62 | | | | | | | | | | | |
| Example | R-63 | A-2 | 7 | C-19 | 100 | Bp-1 | 20 | Bq-1 | 20 | E-1/E-2 | 4800/2000 |
| 63 | | | | | | | | | | | |
| Example | R-64 | A-2 | 7 | C-20 | 100 | Bp-1 | 20 | Bq-1 | 20 | E-1/E-2 | 4800/2000 |
| 64 | | | | | | | | | | | |
| Example | R-65 | A-2 | 7 | C-21 | 100 | Bp-1 | 20 | Bq-1 | 20 | E-1/E-2 | 4800/2000 |
| 65 | | | | | | | | | | | |
| Example | R-66 | A-2 | 7 | C-22 | 100 | Bp-1 | 20 | Bq-1 | 20 | E-1/E-2 | 4800/2000 |
| 66 | | | | | | | | | | | |
| Example | R-67 | A-2 | 7 | C-23 | 100 | Bp-1 | 20 | Bq-1 | 20 | E-1/E-2 | 4800/2000 |
| 67 | | | | | | | | | | | |
| Example | R-68 | A-2 | 7 | C-24 | 100 | Bp-1 | 20 | Bq-1 | 20 | E-1/E-2 | 4800/2000 |
| 68 | | | | | | | | | | | |
| Example | R-69 | A-2 | 7 | C-25 | 100 | Bp-1 | 30 | Bq-1 | 20 | E-1/E-2 | 4800/2000 |
| 69 | | | | | | | | | | | |
| Example | R-70 | A-2 | 7 | C-26 | 100 | Bp-1 | 20 | Bq-1 | 20 | E-1/E-2 | 4800/2000 |
| 70 | | | | | | | | | | | |

TABLE 6

| | | | | | | | | Acid diffusion | | | |
|---|---|---|---|---|---|---|---|---|---|---|---|
| | | | | | | | | control agent | | | |
| | | | | | | | | | ratio (mol %) | | |
| | | | | | | Acid-generating | | | with respect | | |
| | | Polymer (A) | | Polymer (C) | | agent | | | to amount | Solvent | |
| | Radiation- | | Part(s) | | Part(s) | | Part(s) | | of acig- | | Part(s) |
| | sensitive | | by | | by | | by | | generating | | by |
| | composition | Type | mass | Type | mass | Type | mass | Type | agent | Type | mass |
| Example | R-71 | A-2 | 7 | C-27 | 100 | Bp-1 | 20 | Bq-1 | 20 | E-1/E-2 | 4800/2000 |
| 71 | | | | | | | | | | | |
| Example | R-72 | A-2 | 7 | C-28 | 100 | Bp-1 | 20 | Bq-1 | 20 | E-1/E-2 | 4800/2000 |
| 72 | | | | | | | | | | | |
| Example | R-73 | A-2 | 7 | C-29 | 100 | Bp-1 | 20 | Bq-1 | 20 | E-1/E-2 | 4800/2000 |
| 73 | | | | | | | | | | | |
| Example | R-74 | A-2 | 7 | C-30 | 100 | Bp-1 | 20 | Bq-1 | 20 | E-1/E-2 | 4800/2000 |
| 74 | | | | | | | | | | | |
| Example | R-75 | A-2 | 7 | C-31 | 100 | Bp-1 | 20 | Bq-1 | 20 | E-1/E-2 | 4800/2000 |
| 75 | | | | | | | | | | | |
| Example | R-76 | A-2 | 7 | C-32 | 100 | Bp-1 | 20 | Bq-1 | 20 | E-1/E-2 | 4800/2000 |
| 76 | | | | | | | | | | | |
| Example | R-77 | A-2 | 7 | C-33 | 100 | Bp-1 | 20 | Bq-1 | 20 | E-1/E-2 | 4800/2000 |
| 77 | | | | | | | | | | | |
| Example | R-78 | A-2 | 7 | C-34 | 100 | Bp-1 | 20 | Bq-1 | 20 | E-1/E-2 | 4800/2000 |
| 78 | | | | | | | | | | | |
| Example | R-79 | A-2 | 7 | C-35 | 100 | Bp-1 | 20 | Bq-1 | 20 | E-1/E-2 | 4800/2000 |
| 79 | | | | | | | | | | | |
| Example | R-80 | A-2 | 7 | C-36 | 100 | Bp-1 | 20 | Bq-1 | 20 | E-1/E-2 | 4800/2000 |
| 80 | | | | | | | | | | | |
| Example | R-81 | A-2 | 7 | C-2 | 100 | Bp-2 | 20 | Bq-1 | 20 | E-1/E-2 | 4800/2000 |
| 81 | | | | | | | | | | | |
| Example | R-82 | A-2 | 7 | C-2 | 100 | Bp-3 | 20 | Bq-1 | 20 | E-1/E-2 | 4800/2000 |
| 82 | | | | | | | | | | | |
| Example | R-83 | A-2 | 7 | C-2 | 100 | Bp-4 | 20 | Bq-1 | 20 | E-1/E-2 | 4800/2000 |
| 83 | | | | | | | | | | | |
| Example | R-84 | A-2 | 7 | C-2 | 100 | Bp-5 | 20 | Bq-1 | 20 | E-1/E-2 | 4800/2000 |
| 84 | | | | | | | | | | | |
| Example | R-85 | A-2 | 7 | C-2 | 100 | Bp-6 | 20 | Bq-1 | 20 | E-1/E-2 | 4800/2000 |
| 85 | | | | | | | | | | | |
| Example | R-86 | A-2 | 7 | C-2 | 100 | Bp-7 | 20 | Bq-1 | 20 | E-1/E-2 | 4800/2000 |
| 86 | | | | | | | | | | | |
| Example | R-87 | A-2 | 7 | C-2 | 100 | Bp-8 | 20 | Bq-1 | 20 | E-1/E-2 | 4800/2000 |
| 87 | | | | | | | | | | | |
| Example | R-88 | A-2 | 7 | C-2 | 100 | Bp-9 | 20 | Bq-1 | 20 | E-1/E-2 | 4800/2000 |
| 88 | | | | | | | | | | | |
| Example | R-89 | A-2 | 7 | C-2 | 100 | Bp- | 20 | Bq-1 | 20 | E-1/E-2 | 4800/2000 |
| 89 | | | | | | 10 | | | | | |
| Example | R-90 | A-2 | 7 | C-2 | 100 | Bp- | 20 | Bq-1 | 20 | E-1/E-2 | 4800/2000 |
| 90 | | | | | | 11 | | | | | |
| Example | R-91 | A-2 | 7 | C-2 | 100 | Bp- | 20 | Bq-1 | 20 | E-1/E-2 | 4800/2000 |
| 91 | | | | | | 12 | | | | | |
| Example | R-92 | A-2 | 7 | C-2 | 100 | Bp- | 20 | Bq-1 | 20 | E-1/E-2 | 4800/2000 |
| 92 | | | | | | 13 | | | | | |
| Example | R-93 | A-2 | 7 | C-2 | 100 | Bp- | 20 | Bq-1 | 20 | E-1/E-2 | 4800/2000 |
| 93 | | | | | | 14 | | | | | |
| Example | R-94 | A-2 | 7 | C-2 | 100 | Bp | 20 | Bq-1 | 20 | E-1/E-2 | 4800/2000 |
| 94 | | | | | | 15 | | | | | |
| Example | R-95 | A-2 | 7 | C-2 | 100 | Bp- | 20 | Bq-2 | 20 | E-1/E-2 | 4800/2000 |
| 95 | | | | | | 16 | | | | | |
| Example | R-96 | A-2 | 7 | C-2 | 100 | Bp-1 | 20 | Bq-2 | 20 | E-1/E-2 | 4800/2000 |
| 96 | | | | | | | | | | | |
| Example | R-97 | A-2 | 7 | C-2 | 100 | Bp-1 | 20 | Bq-3 | 20 | E-1/E-2 | 4800/2000 |
| 97 | | | | | | | | | | | |
| Example | R-98 | A-2 | 7 | C-2 | 100 | Bp-1 | 20 | Bq-4 | 20 | E-1/E-2 | 4800/2000 |
| 98 | | | | | | | | | | | |
| Example | R-99 | A-2 | 7 | C-2 | 100 | Bp-1 | 20 | Bq-5 | 20 | E-1/E-2 | 4800/2000 |
| 99 | | | | | | | | | | | |
| Example | R-100 | A-2 | 7 | C-2 | 100 | Bp-1 | 20 | Bq-6 | 20 | E-1/E-2 | 4800/2000 |
| 100 | | | | | | | | | | | |
| Example | R-101 | A-2 | 7 | C-2 | 100 | Bp-1 | 20 | Bq-7 | 20 | E-1/E-2 | 4800/2000 |
| 101 | | | | | | | | | | | |
| Example | R-102 | A-2 | 7 | C-2 | 100 | Bp-1 | 20 | Bq-8 | 20 | E-1/E-2 | 4800/2000 |
| 102 | | | | | | | | | | | |
| Example | R-103 | A-2 | 7 | C-2 | 100 | Bp-1 | 20 | Bq-9 | 20 | E-1/E-2 | 4800/2000 |
| 103 | | | | | | | | | | | |
| Example | R-104 | A-2 | 7 | C-2 | 100 | Bp-1 | 20 | Bq-10 | 20 | E-1/E-2 | 4800/2000 |
| 104 | | | | | | | | | | | |
| Example | R-105 | A-2 | 7 | C-2 | 100 | Bp-1 | 20 | Bq-11 | 20 | E-1/E-2 | 4800/2000 |
| 105 | | | | | | | | | | | |

TABLE 7

| | | | | | | | | Acid diffusion | | | |
|---|---|---|---|---|---|---|---|---|---|---|---|
| | | | | | | | | control agent | | | |
| | | | | | | | | | ratio | | |
| | | | | | | | | | (mol %) | | |
| | | | | | | | | | with | | |
| | | | | | | Acid-generating | | | respect to | | |
| | | Polymer (A) | | Polymer (C) | | agent | | | amount | Solvent | |
| | Radiation- | | Part(s) | | Part(s) | | Part(s) | | of acig- | | Part(s) |
| | sensitive | | by | | by | | by | | generating | | by |
| | composition | Type | mass | Type | mass | Type | mass | Type | agent | Type | mass |
| Example | R-106 | A-2 | 7 | C-2 | 100 | Bp-1 | 20 | Bq-12 | 20 | E-1/E-2 | 4800/2000 |
| 106 | | | | | | | | | | | |
| Example | R-107 | A-2 | 7 | C-2 | 100 | Bp-1 | 20 | Bq-13 | 20 | E-1/E-2 | 4800/2000 |
| 107 | | | | | | | | | | | |
| Example | R-108 | A-2 | 7 | C-2 | 100 | Bp-1 | 20 | Bq-14 | 20 | E-1/E-2 | 4800/2000 |
| 108 | | | | | | | | | | | |
| Example | R-109 | A-2 | 7 | C-2 | 100 | Bp-1 | 20 | Bq-15 | 20 | E-1/E-2 | 4800/2000 |
| 109 | | | | | | | | | | | |
| Example | R-110 | A-2 | 7 | C-37 | 100 | Bp-1 | 20 | Bq-1 | 20 | E-1/E-2 | 4800/2000 |
| 110 | | | | | | | | | | | |
| Example | R-111 | A-2 | 7 | C-38 | 100 | Bp-1 | 20 | Bq-1 | 20 | E-1/E-2 | 4800/2000 |
| 111 | | | | | | | | | | | |
| Example | R-112 | A-2 | 7 | C-39 | 100 | Bp-1 | 20 | Bq-1 | 20 | E-1/E-2 | 4800/2000 |
| 112 | | | | | | | | | | | |
| Example | R-113 | A-2 | 7 | C-40 | 100 | Bp-1 | 20 | Bq-1 | 20 | E-1/E-2 | 4800/2000 |
| 113 | | | | | | | | | | | |
| Example | R-114 | A-2 | 7 | C-41 | 100 | — | — | Bq-1 | 20 | E-1/E-2 | 4800/2000 |
| 114 | | | | | | | | | | | |
| Example | R-115 | A-2 | 7 | C-41 | 100 | Bp-1 | 5 | Bq-1 | 20 | E-1/E-2 | 4800/2000 |
| 115 | | | | | | | | | | | |
| Example | R-116 | A-2 | 7 | C-42 | 100 | — | — | Bq-1 | 20 | E-1/E-2 | 4800/2000 |
| 116 | | | | | | | | | | | |
| Example | R-117 | A-2 | 7 | C-43 | 100 | — | — | Bq-1 | 20 | E-1/E-2 | 4800/2000 |
| 117 | | | | | | | | | | | |
| Example | R-118 | A-2 | 7 | C-44 | 100 | — | — | Bq-1 | 20 | E-1/E-2 | 4800/2000 |
| 118 | | | | | | | | | | | |
| Example | R-119 | A-2 | 7 | C-45 | 100 | — | — | Bq-1 | 20 | E-1/E-2 | 4800/2000 |
| 119 | | | | | | | | | | | |
| Example | R-120 | A-2 | 7 | C-46 | 100 | — | — | Bq-1 | 20 | E-1/E-2 | 4800/2000 |
| 120 | | | | | | | | | | | |
| Example | R-121 | A-2 | 7 | C-47 | 100 | — | — | Bq-1 | 20 | E-1/E-2 | 4800/2000 |
| 121 | | | | | | | | | | | |
| Example | R-122 | A-2 | 7 | C-48 | 100 | — | — | Bq-1 | 20 | E-1/E-2 | 4800/2000 |
| 122 | | | | | | | | | | | |
| Example | R-123 | A-2 | 7 | C-49 | 100 | — | — | Bq-1 | 20 | E-1/E-2 | 4800/2000 |
| 123 | | | | | | | | | | | |
| Example | R-124 | A-2 | 7 | C-50 | 100 | — | — | Bq-1 | 20 | E-1/E-2 | 4800/2000 |
| 124 | | | | | | | | | | | |
| Example | R-125 | A-2 | 7 | C-51 | 100 | Bp-1 | 20 | — | — | E-1/E-2 | 4800/2000 |
| 125 | | | | | | | | | | | |
| Example | R-126 | A-2 | 7 | C-52 | 100 | Bp-1 | 20 | — | — | E-1/E-2 | 4800/2000 |
| 126 | | | | | | | | | | | |
| Example | R-127 | A-2 | 7 | C-53 | 100 | Bp-1 | 20 | — | — | E-1/E-2 | 4800/2000 |
| 127 | | | | | | | | | | | |
| Example | R-128 | A-2 | 7 | C-54 | 100 | — | — | Bq-1 | 10 | E-1/E-2 | 4800/2000 |
| 128 | | | | | | | | | | | |
| Comp- | CR-1 | A-44 | 7 | C-2 | 100 | Bp-1 | 20 | Bq-1 | 20 | E-1/E-2 | 4800/2000 |
| arative | | | | | | | | | | | |
| Example | | | | | | | | | | | |
| 1 | | | | | | | | | | | |
| Comp- | CR-2 | A-45 | 7 | C-2 | 100 | Bp-1 | 20 | Bq-1 | 20 | E-1/E-2 | 4800/2000 |
| arative | | | | | | | | | | | |
| Example | | | | | | | | | | | |
| 2 | | | | | | | | | | | |
| Comp- | CR-3 | A-46 | 7 | C-2 | 100 | Bp-1 | 20 | Bq-1 | 20 | E-1/E-2 | 4800/2000 |
| arative | | | | | | | | | | | |
| Example | | | | | | | | | | | |
| 3 | | | | | | | | | | | |
| Comp- | CR-4 | A-2 | 7 | C-2 | 100 | Bp- | 20 | Bq-16 | 20 | E-1/E-2 | 4800/2000 |
| arative | | | | | | 16 | | | | | |
| Example | | | | | | | | | | | |
| 4 | | | | | | | | | | | |

### <Formation of Resist Pattern>

Onto a 12-inch silicon wafer on which an underlayer-film (“AL412”, product of Brewer Science, Inc., thickness: 20 nm) had been formed, each of the above-prepared radiation-sensitive compositions was applied by means of a spin coater (“CLEAN TRACK ACT12”, product of Tokyo Electron Limited). The applied resist composition was subjected to soft baking (SB) at 130° C. for 60 seconds and then cooled at 23° C. for 30 seconds, to thereby form a resist film having a thickness of 50 nm. Subsequently, the resist film was irradiated with EUV light by means of an EUV exposure device (“NXE3300”, product of ASML, NA=0.33, lighting condition: Conventional s=0.89, and mask: imecDEFECT32FFRO2). After the light exposure, the resist film was subjected to post-exposure baking (PEB) at 90° C. for 60 seconds. Then, development of the resist film was performed by use of 2.38 mass % aqueous TMAH solution at 23° C. for 30 seconds, to thereby form a positive-type resist pattern (32 nm line-and-space pattern).

### Evaluation

The above-formed resist patterns were analyzed so as to evaluate the corresponding radiation-sensitive compositions in terms of sensitivity, LWR performance, and the number of development failures through the following procedures. Notably, the measurement of the resist pattern was conducted by means of a scanning electron microscope (“CG-4100”, product of Hitachi High-Tech Corporation).

Tables 8, 9, 10, and 11 show the results.

### [Sensitivity]

In the aforementioned formation of a resist pattern, a dose which can form a 32-nm line-and-space pattern was employed as an optimum exposure dose (Eop), serving as a sensitivity (mJ/cm²). The smaller the value of the sensitivity, the higher the sensitivity (i.e., excellent sensitivity).

### [LWR Performance]

The resist pattern was observed from above under the aforementioned scanning electron microscope. The line width was measured at 50 points selected at random, and the 30 value was determined from the distribution of the measurements. The 3σ value was employed as an LWR index. Regarding LWR performance, the smaller the 3σ value, the more excellent the LWR performance.

### [Number of Development Failures]

A resist film was exposed to light at an optimum exposure dose, and the film was developed, to thereby form a 32-nm line-and-space pattern. The number of failures on the wafer was counted by means of a failure tester (“KLA2810”, product of KLA-Tencor). Also, the failures on the wafer were categorized into failures conceivably attributed to the resist film and exogenous foreign matters. The number of development failures was evaluated as “A” (considerably excellent) when the number of failures conceivably attributed to the resist film was less than 40; “B” (good) when the number was 40 to 50; and “C” (bad) when the number was more than 50.

TABLE 8

| | Radiation- | | | Number of |
|---|---|---|---|---|
| | sensitive | Eop | LWR | development |
| | composition | (mJ/cm²) | (nm) | failures |
| Example 1 | R-1 | 29 | 3.5 | B |
| Example 2 | R-2 | 27 | 3.6 | A |
| Example 3 | R-3 | 26 | 3.9 | A |
| Example 4 | R-4 | 30 | 3.6 | B |
| Example 5 | R-5 | 26 | 3.8 | A |
| Example 6 | R-6 | 25 | 3.9 | B |
| Example 7 | R-7 | 27 | 3.6 | B |
| Example 8 | R-8 | 27 | 3.6 | B |
| Example 9 | R-9 | 27 | 3.6 | B |
| Example 10 | R-10 | 27 | 3.6 | A |
| Example 11 | R-11 | 27 | 3.6 | A |
| Example 12 | R-12 | 26 | 3.7 | A |
| Example 13 | R-13 | 26 | 3.7 | A |
| Example 14 | R-14 | 28 | 3.5 | A |
| Example 15 | R-15 | 29 | 3.5 | A |
| Example 16 | R-16 | 27 | 3.7 | A |
| Example 17 | R-17 | 27 | 3.7 | A |
| Example 18 | R-18 | 30 | 3.5 | B |
| Example 19 | R-19 | 30 | 3.6 | B |
| Example 20 | R-20 | 27 | 3.6 | A |
| Example 21 | R-21 | 27 | 3.6 | A |
| Example 22 | R-22 | 27 | 3.6 | A |
| Example 23 | R-23 | 28 | 3.5 | A |
| Example 24 | R-24 | 28 | 3.5 | A |
| Example 25 | R-25 | 27 | 3.6 | A |
| Example 26 | R-26 | 27 | 3.6 | A |
| Example 27 | R-27 | 28 | 3.5 | A |
| Example 28 | R-28 | 28 | 3.5 | A |
| Example 29 | R-29 | 30 | 3.5 | B |
| Example 30 | R-30 | 26 | 3.7 | A |
| Example 31 | R-31 | 24 | 3.9 | A |
| Example 32 | R-32 | 28 | 3.5 | A |
| Example 33 | R-33 | 27 | 3.6 | A |
| Example 34 | R-34 | 27 | 3.6 | A |
| Example 35 | R-35 | 26 | 3.6 | A |

TABLE 9

| | Radiation- | | | Number of |
|---|---|---|---|---|
| | sensitive | Eop | LWR | development |
| | composition | (mJ/cm²) | (nm) | failures |
| Example 36 | R-36 | 26 | 3.6 | A |
| Example 37 | R-37 | 27 | 3.7 | A |
| Example 38 | R-38 | 26 | 3.7 | A |
| Example 39 | R-39 | 26 | 3.7 | A |
| Example 40 | R-40 | 30 | 3.6 | A |
| Example 41 | R-41 | 25 | 3.6 | A |
| Example 42 | R-42 | 25 | 3.7 | A |
| Example 43 | R-43 | 25 | 3.7 | A |
| Example 44 | R-44 | 25 | 3.7 | A |
| Example 45 | R-45 | 25 | 3.6 | A |
| Example 46 | R-46 | 31 | 3.3 | A |
| Example 47 | R-47 | 26 | 3.9 | A |
| Example 48 | R-48 | 27 | 3.6 | A |
| Example 49 | R-49 | 28 | 3.7 | A |
| Example 50 | R-50 | 28 | 3.8 | A |
| Example 51 | R-51 | 30 | 3.7 | A |
| Example 52 | R-52 | 29 | 3.5 | A |
| Example 53 | R-53 | 25 | 3.9 | A |
| Example 54 | R-54 | 25 | 3.9 | A |
| Example 55 | R-55 | 28 | 3.6 | A |
| Example 56 | R-56 | 27 | 3.7 | A |
| Example 57 | R-57 | 29 | 3.8 | A |
| Example 58 | R-58 | 28 | 3.8 | A |
| Example 59 | R-59 | 25 | 3.9 | A |
| Example 60 | R-60 | 25 | 3.9 | A |
| Example 61 | R-61 | 28 | 3.7 | A |
| Example 62 | R-62 | 26 | 3.7 | A |
| Example 63 | R-63 | 25 | 3.8 | A |
| Example 64 | R-64 | 25 | 3.8 | A |
| Example 65 | R-65 | 26 | 3.7 | A |
| Example 66 | R-66 | 25 | 3.8 | A |
| Example 67 | R-67 | 27 | 3.6 | A |
| Example 68 | R-68 | 27 | 3.5 | A |
| Example 69 | R-69 | 26 | 3.6 | A |
| Example 70 | R-70 | 27 | 3.6 | A |

TABLE 10

| | Radiation- | | | Number of |
|---|---|---|---|---|
| | sensitive | Eop | LWR | development |
| | composition | (mJ/cm²) | (nm) | failures |
| Example 71 | R-71 | 25 | 3.4 | A |
| Example 72 | R-72 | 25 | 3.7 | A |
| Example 73 | R-73 | 26 | 3.6 | A |
| Example 74 | R-74 | 28 | 3.5 | A |
| Example 75 | R-75 | 29 | 3.6 | A |
| Example 76 | R-76 | 25 | 3.7 | A |
| Example 77 | R-77 | 26 | 3.6 | A |
| Example 78 | R-78 | 26 | 3.6 | A |
| Example 79 | R-79 | 26 | 3.6 | A |
| Example 80 | R-80 | 25 | 3.7 | A |
| Example 81 | R-81 | 28 | 3.8 | A |
| Example 82 | R-82 | 27 | 3.6 | A |
| Example 83 | R-83 | 27 | 3.6 | A |
| Example 84 | R-84 | 28 | 3.7 | A |
| Example 85 | R-85 | 28 | 3.7 | A |
| Example 86 | R-86 | 29 | 3.8 | A |
| Example 87 | R-87 | 30 | 3.5 | A |
| Example 88 | R-88 | 30 | 3.8 | A |
| Example 89 | R-89 | 28 | 3.7 | A |
| Example 90 | R-90 | 27 | 3.7 | A |
| Example 91 | R-91 | 26 | 3.8 | A |
| Example 92 | R-92 | 25 | 3.8 | A |
| Example 93 | R-93 | 26 | 3.7 | A |
| Example 94 | R-94 | 25 | 3.7 | A |
| Example 95 | R-95 | 31 | 3.9 | A |
| Example 96 | R-96 | 28 | 3.6 | A |
| Example 97 | R-97 | 26 | 3.6 | A |
| Example 98 | R-98 | 25 | 3.7 | A |
| Example 99 | R-99 | 28 | 3.7 | A |
| Example 100 | R-100 | 27 | 3.6 | A |
| Example 101 | R-101 | 28 | 3.7 | A |
| Example 102 | R-102 | 29 | 3.6 | A |
| Example 103 | R-103 | 27 | 3.7 | A |
| Example 104 | R-104 | 27 | 3.6 | A |
| Example 105 | R-105 | 27 | 3.6 | A |

TABLE 11

| | Radiation- | | | Number of |
|---|---|---|---|---|
| | sensitive | Eop | LWR | development |
| | composition | (mJ/cm²) | (nm) | failures |
| Example 106 | R-106 | 25 | 3.8 | A |
| Example 107 | R-107 | 25 | 3.8 | A |
| Example 108 | R-108 | 26 | 3.9 | A |
| Example 109 | R-109 | 25 | 3.7 | A |
| Example 110 | R-110 | 28 | 3.5 | A |
| Example 111 | R-111 | 30 | 3.4 | A |
| Example 112 | R-112 | 27 | 3.6 | A |
| Example 113 | R-113 | 29 | 3.4 | A |
| Example 114 | R-114 | 28 | 3.8 | A |
| Example 115 | R-115 | 27 | 3.8 | A |
| Example 116 | R-116 | 24 | 3.8 | A |
| Example 117 | R-117 | 29 | 3.7 | A |
| Example 118 | R-118 | 30 | 3.7 | A |
| Example 119 | R-119 | 26 | 3.7 | A |
| Example 120 | R-120 | 28 | 3.8 | A |
| Example 121 | R-121 | 29 | 3.7 | A |
| Example 122 | R-122 | 29 | 3.8 | A |
| Example 123 | R-123 | 27 | 3.7 | A |
| Example 124 | R-124 | 27 | 3.6 | A |
| Example 125 | R-125 | 30 | 3.8 | A |
| Example 126 | R-126 | 27 | 3.8 | A |
| Example 127 | R-127 | 28 | 3.7 | A |
| Example 128 | R-128 | 30 | 3.9 | A |
| Comparative | CR-1 | 34 | 4.5 | C |
| Example 1 | | | | |
| Comparative | CR-2 | 32 | 3.9 | B |
| Example 2 | | | | |
| Comparative | CR-3 | 33 | 4.0 | B |
| Example 3 | | | | |
| Comparative | CR-4 | 35 | 4.5 | A |
| Example 4 | | | | |

As is clear from Tables 8 to 11, the radiation-sensitive compositions employed in the Examples were found to exhibit excellent sensitivity and LWR performance and evoke a small number of development failures. In contrast, the radiation-sensitive compositions employed in the Comparative Examples were found to exhibit at least one properties of sensitivity, LWR performance, and the number of development failures, which was inferior to the same properties of the Examples.

According to the radiation-sensitive composition and resist pattern formation method as described above, there can be formed a resist pattern which exhibits suitable sensitivity to exposure light, excellent LWR performance, and few development failures. Thus, the invention can be suitably applied to formation of a fine resist pattern in a lithography step in production of electronic devices such as semiconductor devices and liquid crystal devices.

1: A radiation-sensitive composition comprising:
(A) a polymer comprising a structural unit represented by the following formula (1); and
(B) an onium salt compound which comprises an organic anion and a cation, in which the organic anion or the cation or both have an iodo group, and which generates an acid through exposure to radiation; 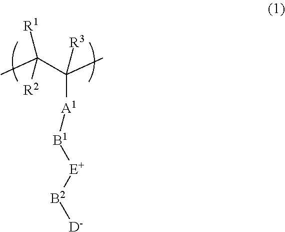
wherein, in the formula (1), each of R¹, R², and R³ independently represents a hydrogen atom, a halogen atom, a hydroxy group, a cyano group, a nitro group, a C1 to C6 alkyl group, or a C1 to C6 haloalkyl group; A¹ represents a single bond, —O—, —CO—, —COO—, —NH—, —CONH—, or *¹—Ar¹-A³-; Ar¹ represents a divalent aromatic ring group; A³ represents a single bond, —O—, —CO—, —COO—, —NH—, or —CONH—; *¹ represents a chemical bond to a carbon atom to which R³ is bound; B¹ represents a single bond or a C≥1 divalent organic group bound to E⁺ in the formula (1) at a carbon atom thereof; E⁺ represents a divalent group comprising an ammonium cation structure or a phosphonium cation structure; B² represents a C≥1 divalent organic group bound to E⁺ and D⁻ in the formula (1) at the same carbon atom or different carbon atoms thereof; and D⁻ represents a monovalent group comprising an anion structure.

2: The radiation-sensitive composition according to claim 1, wherein the compound (B) is represented by following formula (2) or (3); 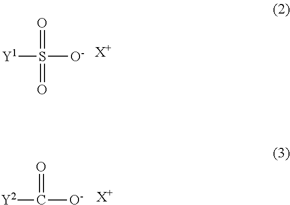
wherein, in the formulas (2) and (3), each of Y¹ and Y² represents a C1 to C40 monovalent organic group; X⁺ represents a monovalent onium cation; in the formula (2), Y¹ or X⁺ or both have an iodo group; and, in the formula (3), Y² or X⁺ or both have an iodo group.

3: The radiation-sensitive composition according to claim 1, wherein the compound (B) comprises a structure in which an iodo group is bound to an aromatic ring.

4: The radiation-sensitive composition according to claim 1, wherein the compound (B) comprises a compound represented by the following formula (2A); 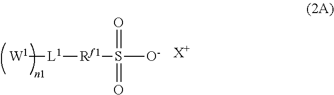
wherein, in the formula (2A), W¹ represents a C5 to C40 monovalent aromatic ring group having an iodo group; L¹ represents a single bond or an (n1+1)-valent organic group; n1 is an integer of 1 or greater; R^{f1} represents an (n1+1)-valent fluorinated hydrocarbon group, when L¹ is a single bond; R^{f1} represents a divalent fluorinated hydrocarbon group, when L¹ is an (n1+1)-valent organic group; and X⁺ represents a monovalent onium cation.

5: The radiation-sensitive composition according to claim 1, wherein the compound (B) comprises a compound represented by the following formula (3A); 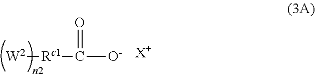
wherein, in the formula (3A), W² represents a C5 to C40 monovalent aromatic ring group having an iodo group; n2 is an integer of 1 or greater; R^{c1} represents a single bond or a divalent organic group, when n2 is 1; R^{c1} represents an (n2+1)-valent organic group, when n2 is 2 or greater; and X⁺ represents a monovalent onium cation.

6: The radiation-sensitive composition according to claim 1, further comprising an onium salt compound which can generate an acid having an acidity lower than an acidity of an acid generated by the compound (B) and which differs from the compound (B).

7: The radiation-sensitive composition according to claim 1, further comprising an onium salt compound which can generate an acid having an acidity higher than an acidity of an acid generated by the compound (B) and which differs from the compound (B).

8: The radiation-sensitive composition according to claim 1, wherein the compound (B) comprises a first onium salt compound, and a second onium salt compound which can generate an acid having an acidity lower than an acidity of an acid generated by the first onium salt compound.

9: The radiation-sensitive composition according to claim 1, wherein the cation comprises a sulfonium cation structure or an iodonium cation structure.

10: The radiation-sensitive composition according to claim 9, wherein the cation comprises an aromatic ring bound to a sulfonium cation or an iodonium cation, and at least one group selected from the group consisting of a fluoroalkyl group, a fluoro group excepting a fluoro group in the fluoroalkyl group, and an iodo group is bound to the aromatic ring.

11: The radiation-sensitive composition according to claim 1, wherein the polymer (A) further comprises a structural unit comprising an acid-dissociable group.

12: The radiation-sensitive composition according to claim 1, wherein the polymer (A) further comprises a structural unit comprising an aromatic group and a hydroxy group bound to the aromatic ring.

13: The radiation-sensitive composition according to claim 1, wherein the polymer (A) further comprises a structural unit comprising a fluorine atom.

14: The radiation-sensitive composition according to claim 13, wherein the polymer (A) comprises the structural unit comprising a fluorine atom in an amount, with respect to all the structural units forming the polymer (A), of 45 to 99 mol %.

15: The radiation-sensitive composition according to claim 13, wherein an amount of the polymer (A), with respect to the entire amount of the composition excepting the solvent, is 0.1 to 20 mass %.

16: The radiation-sensitive composition according to claim 1, wherein the polymer (A) comprises the structural unit represented by the formula (1) in an amount, with respect to all the structural units forming the polymer (A), of 1 to 55 mol %.

17: The radiation-sensitive composition according to claim 1, further comprising
(C) a polymer comprising a structural unit which comprises an acid-dissociable group and the polymer (C) being different from the polymer (A), wherein
the polymer (A) has a fluorine atom content by mass higher than a fluorine atom content of the polymer (C).

18: The radiation-sensitive composition according to claim 1, wherein E⁺ in the formula (1) is represented by the following formula (e-1), (e-2), or (e-3): 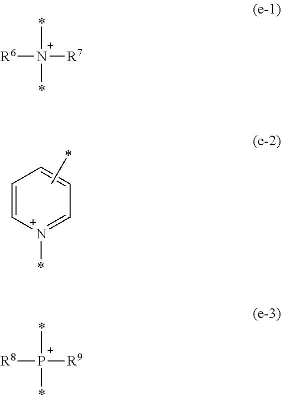
wherein in the formulas (e-1), (e-2) and (e-3), each of R⁶ and R⁷ independently represents a monovalent hydrocarbon group, or R⁶ and R⁷ taken together represent an aliphatic heterocyclic structure with the nitrogen atom to which R⁶ and R⁷ are bound; each of R⁸ and R⁹ independently represents a monovalent hydrocarbon group, or R⁸ and R⁹ taken together represent an heterocyclic structure with the phosphorus atom to which R⁸ and R⁹ are bound; and * represents a chemical bond.

19: The radiation-sensitive composition according to claim 1, wherein D⁻ in the formula (1) is —COO⁻, —SO₃⁻, —PO₃⁻, —POO⁻, or —O⁻.

20: A method of forming a resist pattern, the method comprising
forming a resist film on a substrate by applying the radiation-sensitive composition according to claim 1 onto a substrate,
exposing the resist film to light, and
developing the light-exposed resist film.
